(19) United States
(12) Patent Application Publication
(10) Pub. No.: US 2025/0197484 A1
(43) Pub. Date: Jun. 19, 2025

(54) ANTIBODIES TO NECROTIC ENTERITIS B-LIKE TOXIN

(71) Applicant: ECO ANIMAL HEALTH LTD., London (GB)

(72) Inventors: Charles OWEN, Lindfield, Sussex (GB); Charles GRUMMITT, Lindfield, Sussex (GB); Hafid Abdelaali BENCHAOUI, London (GB); Alfonso Lopez RODRIGUEZ, London (GB)

(21) Appl. No.: 18/866,968

(22) PCT Filed: May 25, 2023
(86) PCT No.: PCT/GB2023/051381

§ 371 Date: Nov. 18, 2024

(30) Foreign Application Priority Data
May 25, 2022 (GB) 2207698.8

(51) Int. Cl. C07K 16/12; A61K 9/00; A61K 39/00; A61P 31/04; C12N 1/20; C12N 15/70; C12R 1/19

(52) U.S. Cl. CPC C07K 16/1282; A61K 9/0053; A61P 31/04; C12N 1/20; C12N 15/70; A61K 2039/505; C07K 2317/14; C07K 2317/565; C07K 2317/569; C07K 2317/76; C07K 2317/92; C07K 2317/94; C12R 2001/19

(57) ABSTRACT

The present disclosure provides a binding protein, e.g. an antibody, comprising at least one antigen binding domain which binds to necrotic enteritis B-like toxin (NetB), said antigen binding domain comprising a heavy chain variable region which comprises three complementarity determining regions (CDRs), preferably wherein said binding protein binds to NetB with a KD of 250 pM or less at pH 7.4. Said binding proteins can be used in the treatment or prevention of C. perfringens infection or Necrotic Enteritis. Nucleic acid molecules, expression vectors, host cells and compositions are also provided.

This invention relates generally to the field of binding proteins which bind to NetB (necrotic enteritis B-like toxin), in particular antibodies, and in particular binding proteins and antibodies that bind to NetB produced by Clostridium perfringens. Such anti-NetB binding proteins and antibodies have therapeutic and protective uses, such as in the treatment or prevention of diseases caused by or associated with the NetB toxin of Clostridium perfringens, in particular Necrotic enteritis (NE), for example reducing its incidence and severity. Binding protein and antibody-based compositions, methods and kits are also provided.

Necrotic enteritis (NE) is a life-threatening gastrointestinal disease that affects animals and poultry such as chickens in particular. The disease is caused by the bacterium Clostridium perfringens, a gram-positive, anaerobic, spore forming pathogenic bacteria. It is ubiquitous in the environment, for example is commonly found in soil, litter, dust and at low levels in the intestine of many humans and animals, e.g. healthy birds (Hatheway, C. L., Clin. Microbiol. Rev. 1990, 3, 66-98).

Avian NE is an enteric disease that is characterized by necrotic lesions in the small intestinal mucosa (Kaldhusdal and Hofshagen, 1992, Poult. Sci., 71, 1145-1153). In poultry, NE is a global problem and it has been estimated that the disease costs the international poultry industry in excess of $US 6 billion per year in production losses and control measures (Cooper and Songer, 2009, Anaerobe, 15, 55-60; Wade et al., 2015, Veterinary Microbiology, 180, 299-303).

Necrotic enteritis occurs when large numbers of Clostridium perfringens grow in the intestines and secrete toxins. These toxins cause necrosis and lesions of the intestines, haemorrhaging, perforation of the intestine, and eventual death from septic shock. NE is a complex disease and predisposing factors that compromise gut integrity are required to facilitate C. perfringens proliferation and toxin production.

There are several different types of C. perfringens, which produce several toxins: alpha, beta, epsilon, iota, CPE and NetB (Rood et al., 2018, Anaerobe, 53: 5-10). All C. perfringens strains produce α-toxin, an extracellular toxin (Awad et al., 1995, Mol. Microbiol., 15, 191-202), but it has been found that this toxin is not necessarily required for NE disease (Keyburn et al., 2006, Infect. Immun., 74, 6496-6500). However, the NetB toxin (which was originally identified in avian C. perfringens type A strains, Keyburn et al., 2008, PLoS Pathog., 4, e6) is believed to be a key, and probably essential, virulence factor in C. perfringens strains that cause avian NE. C. perfringens is the primary causative agent of avian NE, which is typically caused by A-type and C-type strains of C. perfringens (Songer 1996, Clin. Microbiol. Rev., 9, 216-234). Very recently, a new C. perfringens toxin type scheme has been proposed in which the NE-producing strains expressing NetB are currently classified as type G (Rood et al., 2018, Anaerobe, 53: 5-10).

The best current therapeutic option for avian NE is antibiotics. Work has also been carried out to try and develop vaccines based on the toxin molecules. This work has focused mainly on the alpha-toxin, but some work has also been carried out on NetB based vaccines. However, to date, vaccines have only been shown to be partially effective, and, in addition, the available commercial vaccine uses a genetically modified Salmonella vector, which is a potential human safety issue. Antibiotics have shortcomings of exerting selection pressure and causing antimicrobial resistance (in animals and humans).

Thus, there is a clear need for alternative and preferably improved treatment and prevention options for Necrotic enteritis or infections caused by or associated with Clostridium perfringens, in particular infections caused by or associated with the NetB toxin of Clostridium perfringens.

The present invention provides one such alternative therapeutic or preventative option in the form of binding proteins and antibodies (e.g. antibody based binding proteins) directed to NetB, which can act to reduce or prevent Necrotic enteritis (NE), or infections caused by or associated with Clostridium perfringens or NetB toxin, in particular infections caused by or associated with the NetB toxin of Clostridium perfringens, such as NE. An anti-toxin antibody approach advantageously circumvents the use of antibiotics and does not exert selection pressure by virtue of the fact that it does not target the bacteria itself but the toxin which is produced by the bacteria.

Oral delivery of such biologic therapeutics would be a preferred option because it targets the site of infection. However, such modes of delivery are generally prevented due to the degradation of such entities in the acidic and enzyme rich environment of the GI tract and stomach (or more specifically the crop, proventriculus and gizzard in avian species such as chickens).

Antibodies (monoclonal antibodies) of the invention have been shown to be capable of binding to NetB at high affinity and also show good ability to inhibit NetB induced toxicity. Advantageously such antibodies also show these abilities under conditions as found in the GI tract and small intestine. Thus, such abilities are also shown at pH 6.0, as well as pH 7.4. The antibodies are also stable at even lower pH, such as at pH 2.5 or 1.8. In addition, the antibodies show resistance to certain proteases as found in the GI tract and small intestine, in particular chymotrypsin, pepsin and pancreatin. Such properties make these antibodies particularly suitable and advantageous for the therapeutic uses described.

The present inventors have thus provided anti-NetB antibodies that are able to bind to and inhibit the activity or function of NetB, in particular the NetB toxin as produced by Clostridium perfringens. Such antibodies (or for example other binding proteins comprising a NetB antigen binding domain as described herein) can for example inhibit the toxic or cytotoxic ability of NetB, for example inhibit the ability of NetB to lyse or kill cells which are susceptible to the NetB toxin, including the haemolytic activity of NetB, for example on red blood cells. Such antibodies (or for example other binding proteins comprising a NetB antigen binding domain as described herein) can conveniently and advantageously be used to treat or prevent NE in avian species, in particular NE caused by or associated with Clostridium perfringens infection.

In one embodiment, the present invention provides a binding protein, for example an antibody, for example a monoclonal antibody, which binds to NetB, for example Clostridium perfringens NetB, for use in the treatment or prevention of NE, or infections caused by or associated with Clostridium perfringens, in particular infections caused by or associated with the NetB toxin of Clostridium perfringens, such as NE, in a subject (or group of subjects). Preferred subjects are avian species such as poultry.

Thus, in particular, the present invention provides a monoclonal antibody which binds to NetB for use in the treatment or prevention of NE or other infections caused by or associated with the NetB toxin of Clostridium perfringens in poultry. However, the antibodies (or binding proteins) of the present invention can be used in the treatment or prevention of any pathologies in any subject where NetB is shown to play a role, in which case binding and inhibition of this toxic protein would be a useful therapeutic tool.

As discussed elsewhere herein, preferred antibodies (or binding proteins) of the invention and suitable for use in the therapeutic methods described herein have a high affinity for NetB, e.g. when measured by Biacore (the ability to bind NetB is observed at pH 6.0, as well as pH 7.4), the ability to reduce the toxic activity of NetB (this ability is observed at pH 6.0, as well as pH 7.4, and preferably at a temperature of both 42° C. and 37° C.), resistance to various proteases, for example, resistance to pepsin digestion (e.g. at a pepsin concentration of 30 U/ml at a temperature of 37° C.), resistance to chymotrypsin digestion (e.g. at a chymotrypsin concentration of 1 μg/ml at a temperature of 37° C.), and optionally resistance to pancreatin digestion (e.g. at a pancreatin concentration of 0.5 mg/ml at a temperature of 37° C.). The preferred antibodies (or binding proteins) also show stability in water. To the inventors' knowledge no other anti-NetB antibodies have been disclosed to have this advantageous combination of properties and antibodies (or binding proteins) with one or more, preferably all, of these properties are preferred.

In a further embodiment, the present invention provides a binding protein, for example an antibody, comprising at least one antigen binding domain which binds to necrotic enteritis B-like toxin (NetB), for example the NetB toxin as produced by Clostridium perfringens, said antigen binding domain comprising a heavy chain variable region which comprises three complementarity determining regions (CDRs), wherein said heavy chain variable region comprises:
(i) a variable heavy (VH) CDR1 that comprises the amino acid sequence of GSIRSISFMN (SEQ ID NO:2), or a sequence substantially homologous thereto, optionally wherein said substantially homologous sequence is a sequence containing 1, 2, 3 or 4 amino acid substitutions compared to the given CDR sequence,
(ii) a variable heavy (VH) CDR2 that comprises the amino acid sequence of INQIGNT (SEQ ID NO:3), or a sequence substantially homologous thereto, optionally wherein said substantially homologous sequence is a sequence containing 1, 2 or 3 amino acid substitutions compared to the given CDR sequence, and
(iii) a variable heavy (VH) CDR3 that comprises the amino acid sequence of IVRGTVSPFQF (SEQ ID NO:4), or a sequence substantially homologous thereto, optionally wherein said substantially homologous sequence is a sequence containing 1, 2, 3 or 4 amino acid substitutions compared to the given CDR sequence.

In a further embodiment, the present invention provides a binding protein, for example an antibody, comprising at least one antigen binding domain which binds to necrotic enteritis B-like toxin (NetB), for example the NetB toxin as produced by Clostridium perfringens, said antigen binding domain comprising a heavy chain variable region which comprises three complementarity determining regions (CDRs), wherein said heavy chain variable region comprises:
(i) a variable heavy (VH) CDR1 that comprises the amino acid sequence of GLTFSGYSLG (SEQ ID NO:10), or a sequence substantially homologous thereto, optionally wherein said substantially homologous sequence is a sequence containing 1, 2, 3 or 4 amino acid substitutions compared to the given CDR sequence,
(ii) a variable heavy (VH) CDR2 that comprises the amino acid sequence of ISSSGMIT (SEQ ID NO:11), or a sequence substantially homologous thereto, optionally wherein said substantially homologous sequence is a sequence containing 1, 2 or 3 amino acid substitutions compared to the given CDR sequence, and
(iii) a variable heavy (VH) CDR3 that comprises the amino acid sequence of RFPRPRSWLSTDSYNY (SEQ ID NO:12), or a sequence substantially homologous thereto, optionally wherein said substantially homologous sequence is a sequence containing 1, 2, 3 or 4 amino acid substitutions compared to the given CDR sequence.

In a preferred embodiment, the present invention provides a binding protein, for example an antibody, comprising at least one antigen binding domain which binds to necrotic enteritis B-like toxin (NetB), for example the NetB toxin as produced by Clostridium perfringens, said antigen binding domain comprising a heavy chain variable region which comprises three complementarity determining regions (CDRs), wherein said heavy chain variable region comprises:
(i) a variable heavy (VH) CDR1 that comprises the amino acid sequence of GSIRSISFMN (SEQ ID NO:2),
(ii) a variable heavy (VH) CDR2 that comprises the amino acid sequence of INQIGNT (SEQ ID NO:3), and
(iii) a variable heavy (VH) CDR3 that comprises the amino acid sequence of IVRGTVSPFQF (SEQ ID NO:4).

In a preferred embodiment, the present invention provides a binding protein, for example an antibody, comprising at least one antigen binding domain which binds to necrotic enteritis B-like toxin (NetB), for example the NetB toxin as produced by Clostridium perfringens, said antigen binding domain comprising a heavy chain variable region which comprises three complementarity determining regions (CDRs), wherein said heavy chain variable region comprises:
(i) a variable heavy (VH) CDR1 that comprises the amino acid sequence of GLTFSGYSLG (SEQ ID NO:10),
(ii) a variable heavy (VH) CDR2 that comprises the amino acid sequence of ISSSGMIT (SEQ ID NO:11), and
(iii) a variable heavy (VH) CDR3 that comprises the amino acid sequence of RFPRPRSWLSTDSYNY (SEQ ID NO:12).

Certain preferred embodiments of the invention provide an antibody (or binding protein) which binds to necrotic enteritis B-like toxin (NetB), for example the NetB toxin as produced by Clostridium perfringens, comprising a VH domain that has the amino acid sequence of SEQ ID NO: 1 or 9, or a sequence substantially homologous thereto. In some embodiments, such antibodies (or binding proteins) also comprise a VL domain which comprises up to three light chain CDRs, and preferably three light chain CDRs.

In a preferred embodiment the present invention provides an antibody (or binding protein) which binds to necrotic enteritis B-like toxin (NetB), for example the NetB toxin as produced by Clostridium perfringens, comprising a VH domain that has the amino acid sequence of SEQ ID NO: 1 or 9, or a sequence having at least 60%, 65%, 70% or 75% sequence identity thereto, preferably at least 80% sequence identity thereto (e.g. at least 85%, 86%, 87%, 88%, 89%, 90%, 91%, 92%, 93%, 94%, 95%, 96%, 97%, 98% or 99% identity). In some embodiments, such antibodies (or binding proteins) also comprise a VL domain which comprises up to three light chain CDRs, and preferably three light chain CDRs.

In a preferred embodiment, the present invention provides an antibody (or binding protein), which binds to necrotic enteritis B-like toxin (NetB), for example the NetB toxin as produced by Clostridium perfringens, comprising a VH domain that has the amino acid sequence of SEQ ID NO: 1 or 9. In some embodiments, such antibodies (or binding proteins) also comprise a VL domain which comprises up to three light chain CDRs, and preferably three light chain CDRs.

In some embodiments, the antibodies (or binding proteins) are VHH antibodies with three CDRs, or comprise a VH domain with three CDRs, but where no VL regions or domains are present. Thus, in such embodiments, the antigen binding domain may comprise (or consist of) 3 CDR regions and optionally 4 FR regions. In such embodiments, optionally no immunoglobulin or antibody constant regions are present, for example the CDR and optionally the FR regions may be the only antibody derived sequences that are present.

Thus, in some embodiments, the antibodies (or binding proteins), or antigen binding domains thereof, comprise (comprise only) a single heavy chain variable region.

Other preferred embodiments are immunoglobulin (Ig) forms, e.g. IgG, IgA, IgD, IgE or IgM forms, including the chicken IgY form, or forms containing all or part of an immunoglobulin constant region, e.g. all or part of an IgG, or other, constant region, of the various antibodies (or binding proteins) defined herein, for example full length Ig or IgG, IgA or IgY forms. In some embodiments, IgY forms are not used. It is of course understood that full IgG (and other) antibodies will typically comprise two identical or substantially identical heavy chains (with appropriate variable and constant regions) and two identical or substantially identical light chains (with appropriate variable and constant regions). In addition, full length heavy chain only antibodies can also be used, which will typically comprise two identical or substantially identical heavy chains (with appropriate variable and constant regions), such as in camelid antibodies (which typically have two identical or substantially identical heavy chains which comprise a variable region together with CH2 and CH3 constant domains, in particular IgG constant domains such as IgG2 or IgG3 constant domains). Preferred forms containing part of an immunoglobulin constant region are forms containing an Fc region or domain, for example Fc fusions. Such Fc regions or domains are known in the art and generally comprise CH2 and CH3 domains of antibody heavy chains (CH2, CH3 and CH4 domains in the case of IgY and IgE), which associate to form a homodimer. Thus, formats containing CH2 and CH3 domains (or CH2, CH3 and CH4 domains) can be preferred, in particular IgG, IgA or IgY Fc regions, and, in some embodiments, no other antibody constant regions are present.

These regions can be derived from any appropriate source or species, e.g. a source or species which is the same as, or different from, the host species used to generate the antibodies, e.g. by immunization, or a source or species which is the same as, or different from, the source or species from where the antibodies are derived or correspond to. In some embodiments, these regions can correspond to or are derived from Fc regions or domains from the species to which the antibodies are intended to be administered, e.g. for therapy. Appropriate species are defined elsewhere herein, e.g. avian species.

As such Fc regions are homodimeric (or form homodimers), they can conveniently be used to dimerise two polypeptide chains. Thus, by linking or fusing one or more single domain antibodies (e.g. VHH antibodies) of the invention to each chain of an Fc region, when the two chains of the Fc region dimerise they can be used to provide multiple copies of single domain antibodies (e.g. VHH antibodies) of the invention in a single construct or molecule. If more than one single domain antibody (e.g. VHH antibody) of the invention is linked or fused to each chain of an Fc region in sequence then these antibodies can be the same antibody (e.g. two or more copies of the same VHH can be provided on each chain) or different antibodies. Thus, for example, the Fc fusion can be used to provide constructs comprising more than one copies of identical single domain antibodies of the invention or more than one copies of different single domain antibodies of the invention. As such constructs generally contain more than one copy of the same antibody (e.g. more than one copy of one single domain antibody or VHH antibody or more than one copy of multiple different single domain antibody or VHH antibody) of the invention, such constructs may show improved binding of NetB, for example due to an avidity effect.

Binding proteins, e.g. antibodies, based on the Net 83 or Net 14 antibody sequences set forth in Tables A or B, respectively, are preferred. The invention is exemplified by monoclonal antibodies which are VHH antibodies (single domain antibodies), sequences of which are shown in Tables A or B herein. The VH CDR domains and VH domains of each of these VHH antibodies are shown in Tables A or B herein. Antibodies (or binding proteins) comprising these sets of VH CDR domains, or VH domains, or these sets of FR domains, or IgG/Fc containing formats comprising such domains (or sequences substantially homologous thereto) are preferred embodiments of the invention.

Certain examples of substantially homologous sequences are sequences that have at least 60% or 65% identity to the amino acid sequences disclosed. In certain embodiments, the antibodies (or binding proteins) of the invention, or antigen binding domains thereof, comprise one or at least one heavy chain variable region that includes an amino acid sequence region of at least 60%, 65%, 70% or 75%, more preferably at least 80%, more preferably at least 85%, 86%, 87%, 88%, or 89%, more preferably at least 90%, 91%, 92%, 93%, 94%, or 95% and most preferably at least 96%, 97%, 98% or 99% amino acid sequence identity to the amino acid sequence of SEQ ID NO: 1 or 9.

Other preferred examples of substantially homologous sequences are sequences containing conservative amino acid substitutions of the amino acid sequences disclosed.

Other preferred examples of substantially homologous sequences are sequences containing 1, 2, 3 or 4, preferably 1, 2 or 3, preferably 1 or 2 (more preferably 1), altered amino acids in one or more of the CDR regions or one or more of the FR regions disclosed. Such alterations might be conserved or non-conserved amino acid substitutions, or a mixture thereof.

Other preferred examples of “substantially homologous” sequences are sequences having at least 50%, at least 55%, at least 60%, at least 65%, at least 70%, at least 75%, at least 80%, at least 85%, at least 90%, or at least 95%, amino acid sequence identity to the amino acid sequence of one or more of the CDR regions or one or more of the FR regions disclosed in Tables A or B. Thus, in some embodiments, a “substantially homologous” CDR sequence may be a sequence having at least 50%, at least 55%, at least 60%, at least 65%, at least 70%, at least 75%, at least 80%, at least 85%, at least 90% or at least 95% sequence identity to a given CDR sequence described herein.

In some embodiments, in antibodies having a “substantially homologous” sequence as compared to a given sequence, or having a certain degree of sequence identity as compared to a given sequence, the altered amino acid residues(s) are not in a CDR region. For example, in some embodiments, in antibodies having a VH domain that has a certain degree of sequence identity to a given VH domain sequence of a particular antibody of the invention (e.g. Net 14 or Net 83), the altered (or variant) residue(s) are not in a CDR region. Thus, in some embodiments, in antibodies having a “substantially homologous” sequence as compared to a given sequence, or having a certain degree of sequence identity as compared to a given sequence, the altered amino acid residues(s) are in one or more framework regions.

As is evident from elsewhere herein, in other embodiments, in antibodies having a “substantially homologous” sequence as compared to a given sequence, or having a certain degree of sequence identity as compared to a given sequence, the altered amino acid residues(s) may be in a CDR region.

In some embodiments, in an antibody having a “substantially homologous” sequence as compared to a given sequence, or having a certain degree of sequence identity as compared to a given sequence, the three VH CDR amino acid sequences (i.e. all three VH CDR sequences taken together) are considered together to be the whole (or entire) CDR complement of the antibody, and the amino acid sequence of said whole CDR complement of said antibody is at least 70%, preferably at least 80%, or at least 90%, or at least 95% identical to the corresponding whole (or entire) CDR complement of a given starting (or reference) antibody. The starting (or reference) antibody may have the CDR sequences of the Net 14 or Net 83 antibodies of the present invention.

Altered residues might be conserved or non-conserved amino acid substitutions, or a mixture thereof.

In such embodiments, preferred alterations are conservative amino acid substitutions.

In all embodiments, binding proteins, e.g. antibodies, containing substantially homologous sequences retain the ability to bind to NetB, for example the NetB toxin as produced by Clostridium perfringens, or the NetB of SEQ ID NO:17. Preferably, binding proteins, e.g. antibodies, containing substantially homologous sequences retain one or more (preferably all) of the other properties described herein in relation to the Net 83 or Net 14 antibodies as described herein.

Further examples of substantially homologous amino acid sequences in accordance with the present invention are described elsewhere herein.

The CDRs of the antibodies (or binding proteins) of the invention are preferably separated by appropriate framework regions such as those found in naturally occurring antibodies and/or effective engineered antibodies. Thus, the V_{H} (e.g. VHH), V_{L} and individual CDR sequences of the invention are preferably provided within or incorporated into an appropriate framework or scaffold to enable antigen (here NetB) binding. Such framework sequences or regions may correspond to naturally occurring framework regions, FR1, FR2, FR3 and/or FR4, as appropriate to form an appropriate scaffold, or may correspond to consensus framework regions, for example identified by comparing various naturally occurring framework regions. Alternatively, non-antibody scaffolds or frameworks, e.g. T cell receptor frameworks can be used.

Appropriate sequences that can be used for framework regions are well known and documented in the art and any of these may be used. Preferred sequences for framework regions are one or more of the framework regions making up the VHH antibodies of the invention, preferably one or more of the framework regions of the Net 83 or Net 14 VHH antibodies, as disclosed in Tables A or B, or framework regions substantially homologous thereto, and in particular framework regions that allow the maintenance of antigen specificity, for example framework regions that result in substantially the same or the same 3D structure of the antibody.

In certain preferred embodiments, all four of the variable heavy chain (SEQ ID NOs:5, 6, 7 and 8) framework regions (FR), as appropriate, or FR regions substantially homologous thereto, are found in the antibodies (or binding proteins) of the invention.

In other preferred embodiments, all four of the variable heavy chain (SEQ ID NOs:13, 14, 15 and 16) framework regions (FR), as appropriate, or FR regions substantially homologous thereto, are found in the antibodies (or binding proteins) of the invention.

CDR sequences of certain antibodies of the invention are set forth herein in Tables A and B. In some other embodiments, CDR sequences of antibodies of the invention may be CDR sequences in the VH domains and VL domains of antibodies of the invention as identified using any suitable method (or tool), for example as identified according to the well-known methods of Kabat (e.g. Kabat, et al., “Sequences of Proteins of Immunological Interest”, 5th Ed. Public Health Service, National Institutes of Health, Bethesda, MD, 647-669, 1991) or Chothia (e.g. Chothia C, et al. (1989) Nature, 342:877-883, or AI-Lazikani et al., (1997) JMB 273, 927-948), or as identified using the IMGT numbering scheme (e.g. Lefranc, M.-P., The Immunologist, 7, 132-136 (1999); www.imgt.org)). The IMGT numbering scheme has been used to identify the CDR sequences in Tables A and B.

As described above, the present invention provides binding proteins, for example antibodies, or binding proteins comprising the antigen binding domain of an antibody, which bind to (or specifically recognise or specifically bind to) NetB, for example the NetB toxin as produced by Clostridium perfringens. Preferred binding proteins of the invention are antibodies, and in particular VHH antibodies. However, embodiments as described herein which relate to antibodies, e.g. VHH antibodies, apply equally, mutatis mutandis, to other types of binding proteins, or vice versa. Thus, other binding proteins can comprise the antibodies of the invention or can comprise the antigen binding domains of the antibodies of the invention, e.g. the three CDR regions (CDR1, CDR2 and CDR3), or the three CDR regions (CDR1, CDR2 and CDR3) and the four FR regions (FR1, FR2, FR3 and FR4). Multispecific antibodies incorporating the antibodies of the invention also provided, e.g. bispecific or trispecific antibodies.

Preferred binding proteins are any single polypeptide chains which can bind (e.g. specifically bind) to NetB, for example the NetB toxin as produced by Clostridium perfringens. Appropriate types of binding protein which could be used in the invention are known in the art. For example, in some embodiments immunoglobulin based polypeptides are used, which generally comprise CDR regions (and optionally FR regions or an immunoglobulin based scaffold), such that the CDR regions (and optionally FR regions) of the antibodies of the invention can be grafted onto an appropriate scaffold or framework, e.g. an immunoglobulin scaffold.

However, in other embodiments, non-immunoglobulin based scaffolds, e.g. single chain binding scaffolds, can be used onto which appropriate CDRs which mediate target antigen binding can be grafted. For example, the CDR regions (and optionally FR regions) of the antibodies of the invention can be grafted onto an appropriate non-immunoglobulin scaffold and can be selected for the ability to specifically bind to NetB, for example the NetB toxin as produced by Clostridium perfringens. Such molecules are also referred to as antibody mimics (or antibody mimetics). Examples of appropriate non-immunoglobulin based single chain binding proteins are known and described in the art and include fibronectins (or fibronectin-based molecules), for example based on the tenth module of the fibronectin type Ill domain, such as Adnectins (e.g. from Compound Therapeutics, Inc., Waltham, MA); affimers (e.g. from Avacta); ankyrin repeat proteins or DARPins (e.g. from Molecular Partners AG, Zurich, Switzerland); lipocalins, e.g. anticalins (e.g. from Pieris Proteolab AG, Freising, Germany); human A-domains (e.g. Avimers); staphylococcal Protein A (e.g. from Affibody AG, Sweden); thioredoxins; and gamma-B-crystallin or ubiquitin based molecules, e.g. affilins (e.g. from Scil Proteins GmbH, Halle, Germany).

Preferred non-antibody binding proteins (or binding moieties) of the invention have the ability to bind to the same epitope as an anti-NetB antibody of the invention and such binding proteins (or binding moieties) can for example be selected by way of competition assays such as those described elsewhere herein, using for example an antibody of the invention as a reference antibody.

NetB is a known toxin that can be produced by Clostridium perfringens. It has been identified as being a 130 kDa type I pore-forming protein of at or around 322 amino acids in length which has a signal peptide of at or around 30 amino acids in length. Such toxins form pores that disrupt the phospholipid membrane bilayer of both human and animal cells, causing an influx of ions that may lead to cell lysis. NetB has high haemolytic activity towards avian red blood cells.

Of particular relevance to the present invention, NetB can be produced by Clostridium perfringens, and is believed to play a vital role in the ability of this pathogen to cause disease in animals, in particular avian species. In this regard, NetB, and in particular NetB produced by C. perfringens, is believed to be a key causative agent in the disease necrotic enteritis (NE) in animals, in particular avian species such as chickens, turkeys, etc. Thus, the ability of the binding proteins or antibodies of the present invention to bind to NetB and also to inhibit, reduce or otherwise neutralise the toxic activities of NetB are important and preferred.

The binding proteins or antibodies of the present invention thus bind or are capable of binding (or specifically binding) to NetB. In preferred embodiments of the invention the binding proteins or antibodies of the invention bind to or are capable of binding (or specifically binding) to NetB produced by Clostridium perfringens. In preferred embodiments of the invention the binding proteins or antibodies of the invention can inhibit, reduce or otherwise neutralise the toxic activities of NetB.

The NetB to which the antibodies of the present invention bind to or are capable of binding (or specifically recognise or specifically bind) may be from (or may correspond to) any strain or sub-type of Clostridium perfringens, in particular Type A strains of C. perfringens (also referred to as Type G strains).

The binding proteins and antibodies of the invention can bind to any appropriate forms of NetB, in particular toxic forms of NetB. Such forms can thus include full length NetB, or non-full length forms of NetB, for example truncated forms or fragments (functional fragments) of NetB, or other variant forms of NetB which for example retain their toxic ability. Preferred and convenient forms of NetB to which the binding proteins and antibodies of the invention can bind include recombinant NetB, e.g. a recombinant form of Clostridium perfringens NetB, or a native or natural form of NetB, for example a secreted or soluble form of NetB (e.g. as produced by C. perfringens) or a form of NetB when present on the cell surface (cell-surface NetB), for example in cells where NetB has formed a multimeric pore.

Appropriate cell types in which NetB can form a pore will be well known to a person skilled in the art and include intestinal cells.

Exemplary forms of NetB, e.g. recombinant NetB, as can be used herein in order to assess binding capability of the binding proteins and antibodies are full length NetB (with or without the signal peptide, conveniently without the signal peptide), preferably full length NetB corresponding to the (toxic) NetB protein produced by Clostridium perfringens, or inactive mutant forms of NetB such as a toxoid mutant form, for example the W262A mutant form which is well known and described in the art.

The sequences of NetB are well known and described in the art and can be obtained for example from various sequence databases, e.g. Uniprot. For ease of reference, the Clostridium perfringens NetB has the Uniprot number A8ULG6 or D5K6C0 or EU143239.

A preferred and exemplary NetB molecule has or comprises (or consists of) the sequence EU143239 or SEQ ID NO:17 (signal peptide removed). Thus, preferred binding proteins or antibodies of the invention bind to or are capable of binding (or specifically binding) to SEQ ID NO:17, or a sequence substantially homologous thereto (e.g. with at least 80% identity thereto), or a fragment, e.g. a biologically active fragment, thereof. Preferred biologically active fragments of NetB have or retain their toxic activity. However, some non-toxic or inactive forms, e.g. mutant or variant forms, of NetB are known in the art, e.g. toxoid mutant forms of NetB, and in some embodiments the binding proteins or antibodies of the invention can also bind to these forms of NetB. A particular example of an inactive form of NetB is the W262A mutant form (numbering without signal peptide, SEQ ID NO:18) that can be used to immunise animals, e.g. in vaccination protocols.

Methods of assessing binding to (or ability to bind to) appropriate forms of NetB would be well-known to a person skilled in the art and any appropriate method can be used.

A convenient and appropriate method for assessing binding would include in vitro binding assays such as ELISA assays to assess binding of antibodies to immobilised antigen, such as immobilised forms of NetB as described above, e.g. monomeric NetB, e.g. comprising SEQ ID NO:17 or a mutant form thereof, e.g. the W262A mutant form. The skilled person will be familiar with ELISA assays and readily able to establish suitable conditions to assess the ability of a binding protein or antibody to bind to NetB in such an assay. A particularly preferred ELISA assay (binding ELISA) is described in the Examples section.

In certain embodiments, binding proteins or antibodies of the present invention bind to NetB (e.g. recombinant NetB or Net B corresponding to Clostridium perfringens NetB, e.g. with SEQ ID NO:17) in (as determined in) a Surface Plasmon Resonance (SPR) assay (e.g. a BIACore assay). Suitable SPR assays are known in the art. In certain preferred SPR assays, an appropriate form of NetB, e.g. NetB, e.g. monomeric NetB, with SEQ ID NO:17, or a mutant form thereof, e.g. the W262A mutant form, is captured (or immobilised) on a solid support (e.g. a sensor chip), for example via amine coupling (e.g. approximately 350 Response Units (RU) NetB is immobilized) and various concentrations (e.g. a dilution series, e.g. a doubling or trebling dilution series) of the binding protein or antibody to be tested is then injected. Antibody concentrations are generally selected at a range such that the chip is not saturated and which allow robust fitting by the SPR/Biacore software. Preferred concentrations and flow-rates for injection are described in the Examples section.

Such SPR assay methods can also conveniently be used to measure the binding kinetics of the antibody-antigen interaction, e.g. to determine association rate (ka), dissociation rate (kd) and affinity (KD). In certain embodiments, measurements may be performed at 25° C. in a suitable buffer, e.g. a standard HEPES-EDTA buffer such as HBS-EP (sold by GE Healthcare Life Sciences, 0.01M HEPES pH 7.4, 0.15M NaCl, 3 mM EDTA, 0.0005% surfactant P20), at pH7.4. Kinetic parameters may be determined or calculated by any suitable model or software, for example by fitting the sensogram experimental data assuming a 1:1 interaction, for example using the Single Cycle Kinetics predefined evaluation method of the Biacore Insight Evaluation software. A particularly preferred SPR assay is described in the Examples section herein.

Thus, in a particularly preferred embodiment, binding proteins or antibodies of the present invention bind to NetB (e.g. recombinant NetB or Net B corresponding to Clostridium perfringens NetB, e.g. with SEQ ID NO:17) in (as determined in, when assessed in) a Surface Plasmon Resonance (SPR) assay (e.g. a BIACore assay). Preferred SPR assays and conditions for such measurements are described elsewhere herein. Exemplary binding affinities are also described elsewhere herein.

In certain preferred embodiments, antibodies of the present invention, when in VHH format, have a high binding affinity for NetB (e.g. recombinant NetB or Net B corresponding to Clostridium perfringens NetB, e.g. with SEQ ID NO:17), have a K_{D} (equilibrium dissociation constant) in the range of 1 nM or 500 pM or lower (better), for example when determined in an SPR assay.

Thus, preferably, antibodies of the invention, when in VHH format, have a binding affinity for NetB (e.g. recombinant NetB or Net B corresponding to Clostridium perfringens NetB, e.g. with SEQ ID NO:17) that corresponds to a K_{D} of less than 10 nM, less than 1 nM (or less than 1000 pM), less than 900 pM, less than 800 pM, less than 700 pM, less than 600 pM, less than 500 pM, less than 450 pM, less than 400 pM, less than 350 pM, less than 300 pM, less than 250 pM, less than 225 pM, less than 200 pM, less than 180 pM, less than 160 pM, less than 150 pM, less than 140 pM, less than 130 pM, less than 120 pM, less than 110 pM, less than 100 pM, less than 90 pM, less than 80 pM, less than 70 pM, or less than 60 pM, for example when determined in an SPR assay. Particular exemplary binding affinities are disclosed in the Examples. For example, the exemplified Net 83 antibody of the invention shows a binding affinity of 213 pM and the Net 14 antibody of the invention shows a binding affinity of 95 pM at a pH of 7.4. Thus, preferred binding proteins or antibodies of the invention comprise an antigen binding domain, or at least one antigen binding domain, which binds to NetB with a KD of 250 pM or less (or less than 250 pM) at pH 7.4. Other exemplary KDs are provided above. Put another way, the binding proteins, e.g. antibodies, of the invention can bind to NetB with a KD of 250 pM or less at pH 7.4.

Thus, in one embodiment, the present invention provides a binding protein, e.g. an antibody, comprising an (or at least one) antigen binding domain which binds to necrotic enteritis B-like toxin (NetB), said antigen binding domain comprising a (or at least one) heavy chain variable region which comprises three complementarity determining regions (CDRs), preferably wherein said binding protein (or antigen binding domain) binds to NetB with a KD of 250 pM or less at pH 7.4.

Exemplary forms of NetB that can be used to assess such binding affinity are recombinant NetB, e.g. with a sequence of SEQ ID NO:17 Appropriate exemplary forms are described in the Examples section. Thus, the binding affinities above may be observed when or if the antibodies of the invention are assayed using these sequences, e.g. in an SPR assay. Preferred SPR assays and conditions for such measurements, e.g. immobilized NetB concentrations, buffer, and antibody concentrations, are described elsewhere herein, including in the Examples section. An exemplary pH to be used would be pH 7.4.

Antibodies (or binding proteins) of the invention are capable of binding to NetB, e.g. as described above, at a neutral pH or a pH level as found in neutral biological fluids such as serum or blood. Thus, antibodies of the invention are capable of binding to NetB, e.g. as described above, at a pH of at or around 7.4.

Advantageously, antibodies (or binding proteins) of the invention are also capable of binding to NetB, e.g. as described above, at low or lower pHs, e.g. a pH level such as that found in the gastrointestinal tract or small intestine. For example, pH levels in the gastrointestinal tract or small intestine are believed to range from around 5.7 to 6.5. Thus, antibodies of the invention are capable of binding to NetB, e.g. as described above, e.g. at the binding affinities as described above, at a pH of at or around pH 6.0. This property is advantageous as it means that the antibodies of the invention can continue to bind to the NetB target antigen in the conditions found in the gastrointestinal tract or small intestine, which is a key site of action of the NetB toxin, for example when this toxin is produced by C. perfringens. This property is also advantageous for oral administration, or other administration routes where the antibody is delivered to the gastrointestinal tract or small intestine.

In preferred embodiments of the invention, antibodies (or binding proteins) show similar or improved levels of binding at pH 6.0 to pH 7.4.

As well as being capable of binding to NetB, antibodies (or binding proteins) of the invention are capable of inhibiting (or reducing or neutralising or blocking) the toxic (or cytotoxic) effect or activity of the NetB toxin, e.g the NetB toxin as produced by C. perfringens. For example, antibodies (or binding proteins) of the invention are capable of inhibiting (or reducing or neutralising or blocking) the ability of NetB to cause lysis or haemolysis of cells, for example appropriate target cells such as red blood cells.

Preferably, the inhibition or reduction is a measurable inhibition or reduction, more preferably a significant inhibition or reduction, e.g. a statistically significant inhibition or reduction such as with a probability value of 50.05 or <0.05. In certain embodiments, binding proteins or antibodies of invention can inhibit or reduce (or neutralise) the ability of NetB, e.g. NetB as produced by C. perfringens, to cause haemolysis of cells by at least 60%, at least 70%, at least 75%, at least 80%, at least 85%, at least 90%, at least 92%, at least 95% or at least 98%. Preferred antibodies of the invention have the ability to almost completely inhibit NetB toxicity, for example NetB induced haemolysis, for example at least 90% inhibition can be observed. Typically, such % inhibition (and other percentage inhibition levels as described herein) is in comparison with (or relative to) an appropriate control assay or control level, for example a control assay or control level in the absence of a binding protein or antibody (anti-NetB antibody), for example a negative control or background level or assay. Thus, a 0% inhibition (control) level (or conversely a 100% or maximum lysis level) is typically the level in the absence of a binding protein or antibody (anti-NetB antibody).

Such ability to inhibit (or reduce or neutralise) the toxic effect or activity of the NetB toxin, or to inhibit, etc., NetB induced toxicity, e.g the NetB toxin produced by C. perfringens, can be determined or tested in any appropriate assay, examples of which would be readily derived by a person skilled in the art. Appropriate assays might for example be in vitro assays and for example involve the use of appropriate target cells for NetB toxin, and measuring the effect of the antibodies (or binding proteins) of the invention to inhibit (or reduce or neutralise) the toxic effect or activity of the NetB toxin on such cells.

Particularly useful assays can involve the measurement of the ability of the antibodies (or binding proteins) of the invention to inhibit (or reduce or neutralise) the haemolytic effect of the NetB toxin on target cells such as red blood cells, in particular chicken red blood cells. Such assays can also be referred to as NetB inhibition assays or red blood cell lysis assays, e.g. chicken red blood cell lysis assays. In such assays, appropriate cells (e.g. red blood cells such as chicken red blood cells) can be brought into contact with NetB at a level which will cause haemolysis, for example complete/maximum haemolysis, or sub-maximal (e.g. 80% lysis or an EC₈₀ dose or concentration of NetB) of the cells. Ability of the binding proteins or antibodies of the invention to inhibit or reduce such haemolysis can then readily be analysed, for example in comparison with (or relative to) a 100% haemolysis level set by the control assay. Such ability can conveniently be assessed by calculating an IC₅₀ value. For the avoidance of doubt, the term IC₅₀ value as used herein refers to the concentration of a binding protein or antibody required to inhibit 50% of the toxic/cytotoxic activity of the NetB which is present. An appropriate and exemplary haemolysis assay using chicken red blood cells is described in the Examples section.

Any appropriate concentrations of binding protein or antibody may be used to inhibit or reduce the toxic or haemolytic effect. Exemplary antibodies of the invention have the ability to cause inhibition, e.g. the levels of inhibition as outlined herein, with antibody, in particular VHH, when used at concentrations of at least 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5 or 5.0 μg/ml, e.g. at concentrations up to 1.0, 1.5, 1.8, 2.0, 3.0, 4.0, or 5.0 μg/ml, e.g. between 0.3, 0.4 or 0.5 and 1.0, 1.5, 1.8 or 2.0 μg/ml, or between 0.3, 0.4 or 0.5 and 3.0, 4.0 or 5.0 μg/ml.

It can be noted that the binding protein or antibody of the invention targets the NetB toxin itself, as opposed to the NetB producing entity, e.g. the infectious entity such as C. perfringens. This provides an important advantage of being able to inhibit or reduce disease or symptoms caused by infection with any entity which produces NetB (e.g. NetB as described elsewhere herein), or any form of NetB (or other protein) that can be bound by the antibodies (or binding proteins) of the invention. In this way, the antibodies, etc., of the invention can provide a means for treating disease caused by many strains of C. perfringens and other infectious agents which produce NetB or similar toxins, providing such infectious agents use such toxins to cause disease (in other words have NetB or similar toxins as a causative agent of disease). Thus, the antibodies, etc., of the present invention have wide utility and can for example be used to treat or prevent any disease in which NetB (or a similar toxin to which the antibodies of the invention can bind) is a causative agent. They can also be used to treat early stage disease, as they target the toxic agent produced by the C. perfringens rather than the C. perfringens entity itself. Thus, the antibodies of the invention provide important advantages and flexibility over various prior methods such as vaccination.

In certain embodiments, antibodies (or binding proteins) of the present invention have an IC₅₀ (e.g. for the inhibition or reduction or neutralisation of NetB, for example inhibition of NetB haemolysis of target cells, e.g. red blood cells, preferably chicken red blood cells) of 300 nM or less, 280 nM or less, 260 nM or less, 250 nM or less, 240 nM or less, 220 nM or less, 210 nM or less, 200 nM or less, 190 nM or less, 180 nM or less, 170 nM or less, 160 nM or less, 150 nM or less, 140 nM or less, 130 nM or less, 120 nM or less, 110 nM or less, 100 nM or less, 90 nM or less, 80 nM or less, 70 nM or less, 60 nM or less, 50 nM or less, 40 nM or less, 35 nM or less, 30 nM or less, 25 nM or less, or 20 nM or less. In some embodiments, the IC₅₀ is 20 or 30 to 300, 250 or 200 nM; or 20 or 30 to 150 nM; or 20 or 30 to 140 nM; or 20 or 30 to 130 nM or 20 or 30 to 120 nM; or 20 or 30 to 110 nM; or 20 or 30 to 100 nM; or 20 or 30 to 90 nM; or 20 or 30 to 80 nM; or 20 or 30 to 70 nM. Particular exemplary IC₅₀ values are also shown in the Examples. For example, Net 14 has shown an IC₅₀ of as low as 23 nM and Net 83 has shown as IC₅₀ of as low as 61 nM.

In certain embodiments, antibodies (or binding proteins) of the present invention have an IC₅₀ (e.g. for the inhibition or reduction or neutralisation of NetB, for example inhibition of NetB haemolysis of target cells, e.g. red blood cells, preferably chicken red blood cells), which, when expressed as a molar ratio to NetB toxin used in the assay, is a ratio of about 7:1, 6:1, 5:1, 4:1, 3:1, 2:1 or 1:1 (antibody:NetB). Thus, the antibodies (or binding proteins) of the invention are extremely potent. For example, Net 14 has shown a molar ratio of about 1:1 and Net 83 has shown a molar ratio of about 2:1 or 3:1.

The preferred IC₅₀ values as described above are preferably as determined in an appropriate toxicity assay carried out under appropriate conditions to enable IC₅₀ values to be measured or determined, e.g. an appropriate haemolysis assay, for example as described above or in the Examples section. These assays can be carried out with any appropriate antibody (or binding protein) format. Thus, the exemplary values provided above can for example be values as determined when a VHH or alternative single domain format of the antibodies is assessed.

The NetB inhibition assays as described herein can be carried out at any appropriate pH, for example at the pHs described elsewhere herein, typically at a neutral physiological pH, e.g. pH 7.4, or at a pH typical of the gastrointestinal tract or small intestine, e.g. at a pH of from 5.7 to 6.5, e.g. pH 6.0. Appropriate amounts (doses) of NetB can also be used and these can be readily determined by a person skilled in the art. For example, amounts (doses) of NetB that can cause a certain amount of lysis (e.g. as determined in a separate NetB-lysis titration, e.g. a NetB RBC-lysis titration), e.g. maximal (100%) lysis/toxicity or a different sub-maximal % lysis/toxicity can be chosen, for example 80% lysis/toxicity (i.e. an EC₈₀ value or dose).

The exemplary values provided above can for example be values as determined when NetB is used at an EC₈₀ concentration or dose, for example at a concentration of 20 nM. Thus, the exemplary values provided above can for example be values as determined when NetB is used at a concentration of 20 nM and/or when the assay is carried out at a pH of 7.4 or 6.0. Thus, the exemplary values provided above can for example be values as determined when NetB is used at an EC₈₀ concentration or dose (e.g. a concentration of 20 nM) and/or when the assay is carried out at a pH of 7.4 or 6.0. Thus, for example the binding proteins or antibodies of the invention are capable of reducing the toxic activity of NetB at a pH of 6.0. For example, the binding proteins or antibodies of the invention are capable of reducing the toxic activity of an EC₈₀ concentration of NetB or 20 nM NetB against chicken red blood cells with an IC₅₀ value of less than 150 nM (or 150 nM or less) at a pH of 6.0. Other exemplary IC₅₀ values are provided herein.

Preferred antibodies (or binding proteins) of the invention show equivalent, but more preferably, better activity, e.g. a lower or reduced IC₅₀ value, when assayed at pH 6.0 when compared to a pH of 7.4. Such antibodies should be particularly well adapted to be effective against NetB in the conditions found in the gastrointestinal tract or small intestine, which is advantageous when it comes to the treatment of diseases caused by or associated with NetB in this location of the body, e.g. for diseases caused by C. perfringens infection, and in particular diseases caused by production of NetB by C. perfringens, such as NE. For example, Net 83 and Net 14 both show this improved activity/lower IC₅₀ value.

In alternative embodiments of the invention, the binding proteins or antibodies of the invention can be used to reduce the risk of or prevent diseases caused by or associated with NetB in this location of the body, e.g. for diseases caused by C. perfringens infection, and in particular diseases caused by production of NetB by C. perfringens, such as NE.

The antibodies (or binding proteins) of the invention advantageously and preferably exhibit the further property of being resistant to proteases, in particular to various proteases as found in the gastrointestinal and small intestine environment. Not all antibodies which exhibit the ability to bind to NetB display these properties and thus again such antibodies are particularly well adapted to be effective against NetB in the conditions found in the gastrointestinal tract or small intestine.

For example, antibodies (or binding proteins) of the invention are resistant to chymotrypsin or resistant to chymotrypsin digestion. The term “resistant to chymotrypsin” or “resistant to chymotrypsin digestion” as used herein includes that the antibodies (or binding proteins) do not lose, or maintain, a significant or measurable ability to bind to NetB and/or to inhibit (or reduce or neutralise) the toxic activity of NetB as described elsewhere herein, after exposure to or contact with chymotrypsin. For example, antibodies with this property are capable of inhibiting (or reducing or neutralising) the ability of NetB to cause haemolysis of cells, for example appropriate target cells such as red blood cells, after contact with (or exposure to) chymotrypsin. Such antibodies are also capable of binding to NetB, for example NetB as produced by C. perfringens, after contact with (or exposure to) chymotrypsin. Viewed alternatively, such antibodies retain activity to inhibit (or reduce or neutralise) NetB after coming into contact with (or exposure to) chymotrypsin. These abilities of the antibodies after exposure to chymotrypsin (or in the presence of chymotrypsin) can conveniently be compared to the level observed in the absence of chymotrypsin, or without exposure to chymotrypsin.

Any appropriate level or concentration of chymotrypsin can be used. Preferred and exemplary levels would reflect those as found in the small intestine or GI tract. An exemplary level disclosed herein is a concentration of 1 μg/ml chymotrypsin. Other exemplary exposure conditions would be a temperature of 37° C. for an appropriate time period, e.g. for or for at least 30 minutes and for example up to 2 hours or up to one hour. An exemplary pH would be a pH of 7.4. Thus, preferred antibodies (or binding proteins) of the invention are resistant to chymotrypsin digestion at a chymotrypsin concentration of 1 μg/ml, for example at 37° C.

Preferred antibodies of the invention are resistant to chymotrypsin digestion at a chymotrypsin concentration of 1 μg/ml, when exposed to said chymotrypsin for or for at least 30 minutes, for example one hour, for example at 37° C. Each of the exemplified antibodies, Net 14 and Net 83, display this property.

In certain embodiments, after exposure to chymotrypsin, binding proteins or antibodies of invention can still inhibit or reduce (or neutralise) the ability of NetB, e.g. NetB as produced by C. perfringens, to cause haemolysis of cells by at least 30%, at least 40%, at least 50%, at least 55%, at least 60%, at least 70%, at least 75%, at least 80%, at least 85%, at least 90%, at least 92%, at least 95% or at least 98%. Preferred antibodies of the invention have the ability to almost completely inhibit NetB toxicity, for example NetB induced haemolysis, after exposure to chymotrypsin, for example at least 85% or 90% inhibition can be observed. Alternatively, at least 50%, 60%, 70%, 75% or 80% inhibition can be observed.

Typically, such % inhibition (and other percentage inhibition levels as described herein) is in comparison with (or relative to) an appropriate control assay or control level, for example a control assay or control level in the absence of a binding protein or antibody (anti-NetB antibody), for example a negative control or background level or assay. Thus, a 0% inhibition (control) level (or conversely a 100% or maximum haemolysis level) is typically the level in the absence of a binding protein or antibody (anti-NetB antibody).

In addition, such % inhibition after exposure to chymotrypsin (in the presence of chymotrypsin), can also conveniently be compared to an equivalent % inhibition in the absence of chymotrypsin in order to assess or determine the resistance or relative resistance of the antibody (or binding protein) to chymotrypsin. Preferred antibodies (or binding proteins) of the invention maintain a significant or measurable ability to bind to NetB and/or to inhibit (or reduce or neutralise) the toxic activity of NetB as described elsewhere herein, after exposure to chymotrypsin, e.g. exposure to chymotrypsin as described herein. For example, the ability of such antibodies (or binding proteins) to bind to NetB and/or to inhibit (or reduce or neutralise) the toxic activity of NetB is not significantly reduced by exposure to chymotrypsin.

In certain embodiments, following exposure to chymotrypsin, antibodies (or binding proteins) of the present invention have an IC₅₀ (e.g. for the inhibition, reduction, or neutralisation of NetB, for example inhibition of NetB haemolysis of target cells, e.g. red blood cells, preferably chicken red blood cells) of 300 nM or less, 280 nM or less, 260 nM or less, 250 nM or less, 240 nM or less, 220 nM or less, 210 nM or less, 200 nM or less, 190 nM or less, 180 nM or less, 170 nM or less, 160 nM or less, 150 nM or less, 140 nM or less, 130 nM or less, 120 nM or less, 110 nM or less, 100 nM or less, 90 nM or less, 80 nM or less, 70 nM or less, 60 nM or less, 50 nM or less, 40 nM or less, 35 nM or less, 30 nM or less, 25 nM or less, or 20 nM or less. In some embodiments, the IC₅₀ is 20 or 30 to 300, 250 or 200 nM; or 20 or 30 to 160 nM; or 20 or 30 to 150 nM; or 20 or 30 to 120 nM; or 20 or 30 to 110 nM. Preferably, following exposure to chymotrypsin, antibodies (or binding proteins) of the invention have an IC₅₀ of 300 nM or less, 260 nM or less, 240 nM or less, 220 nM or less, 210 nM or less, 200 nM or less, 190 nM or less, 180 nM or less, 170 nM or less, 160 nM or less, 150 nM or less, 140 nM or less, 130 nM or less, 120 nM or less. Particular exemplary IC₅₀ values and assay conditions are described elsewhere herein or are shown in the Examples. For example, the exemplified antibody Net 14 still shows an IC₅₀ of 114 nM and Net 83 still shows an IC₅₀ of 195 nM after exposure to 1 μg/ml chymotrypsin for 30 minutes or 1 hour at 37° C. Thus, in some embodiments antibodies (or binding proteins) of the invention have an IC₅₀ of 220 nM or less after exposure to 1 μg/ml chymotrypsin for 30 minutes or 1 hour at 37° C. In such embodiments an exemplary pH is 7.4. In such embodiments an exemplary concentration of NetB is an EC₈₀ concentration or dose, or a concentration of 20 nM. Other exemplary IC₅₀ values are described elsewhere herein.

Appropriate assays to assess the ability to bind to NetB and/or to neutralise (or inhibit or reduce) the toxic activity of NetB are well known in the art and any of these may be used. Preferred and exemplary assays are described elsewhere herein, for example haemolytic assays, or ELISA or SPR binding assays.

Thus, the preferred IC₅₀ values as described above are preferably as determined in an appropriate toxicity assay (NetB inhibition assay) carried out under appropriate conditions to enable IC₅₀ values to be measured or determined as described elsewhere herein. Thus, the exemplary values provided above can for example be values as determined when the antibodies, preferably VHH antibodies (or binding proteins) of the invention are exposed to 1 μg/ml chymotrypsin for 30 minutes or 1 hr at 37° C., before being assessed in a toxicity assay (NetB inhibition assay) using an appropriate concentration (toxic concentration) of NetB (e.g. an EC₈₀ concentration or dose), preferably a haemolytic assay, more preferably a haemolytic assay where lysis of chicken red blood cells is assessed, for example using a NetB concentration of 20 nM. An exemplary pH would be a pH of 7.4. Thus, the exemplary values provided above can for example be values as determined when NetB is used at an EC₈₀ concentration (e.g. a concentration of 20 nM) and/or when the assay is carried out at a pH of 7.4.

Preferred antibodies (or binding proteins) of the invention retain at least 50%, e.g. at least 55%, 60%, 65%, 70%, 75%, 80% or 85% ability to inhibit or neutralise NetB, when exposed to chymotrypsin (or in the presence of chymotrypsin), in comparison with the same antibody (or binding protein) which has not been exposed to chymotrypsin (or in the absence of chymotrypsin). Again, such values can be values as determined when the antibodies, preferably VHH antibodies (or binding proteins) of the invention are exposed to 1 μg/ml chymotrypsin for 1 hr at 37° C., before being assessed in a toxicity assay (NetB inhibition assay) using an appropriate concentration (toxic concentration) of NetB, preferably a haemolytic assay, more preferably a haemolytic assay where lysis of chicken red blood cells is assessed for example using a NetB concentration of 20 nM. Particular exemplary % retention values and assay conditions are also shown in the Examples. For example, the exemplified antibody Net 14 retains 66% activity and Net 83 retains 80% activity after exposure to chymotrypsin. Thus, in some embodiments, antibodies (binding proteins) with these, or at least these, % retention values, under these conditions are preferred.

In some embodiments, antibodies (or binding proteins) of the invention are resistant to pepsin or resistant to pepsin digestion. The term “resistant to pepsin” or “resistant to pepsin digestion” as used herein includes that the antibodies (or binding proteins) do not lose, or maintain, a significant or measurable ability to bind to NetB and/or to inhibit (or reduce or neutralise) the toxic activity of NetB as described elsewhere herein, after exposure to or contact with pepsin. For example, antibodies with this property are capable of inhibiting (or reducing or neutralising) the ability of NetB to cause haemolysis of cells, for example appropriate target cells such as red blood cells, after contact with (or exposure to) pepsin. Such antibodies are also capable of binding to NetB, for example NetB as produced by C. perfringens, after contact with (or exposure to) pepsin. Viewed alternatively, such antibodies retain activity to inhibit (or reduce or neutralise) NetB after coming into contact with (or exposure to) pepsin. These abilities of the antibodies after exposure to pepsin (or in the presence of pepsin) can conveniently be compared to the level observed in the absence of pepsin, or without exposure to pepsin.

Any appropriate level or concentration of pepsin can be used. Preferred and exemplary levels would reflect those as found in the small intestine or GI tract. An exemplary level disclosed herein is a concentration of 30 U/ml (corresponding to at or around 30 μg/ml) pepsin. However, higher levels of at least 100 U/ml (corresponding to at or around at least 100 μg/ml) have also been used, in particular a concentration of 107 U/ml (corresponding to at or around 107 μg/ml). Other exemplary exposure conditions would be a temperature of 37° C. for an appropriate time period, e.g. for or for at least 5, 15, 30, or 45 minutes and for example up to 2 hours, up to 1.5 hours, or up to one hour. As pepsin is only active at acidic pH (for example it is inactive at neutral physiological pHs such as pH 7.4), another exemplary exposure condition is that of acidic pH, in particular a pH of 1.8 or 2.5, or a pH in between, or a pH of below 3.0 or below 2.0, is appropriate.

Thus, preferred antibodies of the invention are resistant to pepsin digestion at a pepsin concentration of 30 U/ml or 107 U/ml, for example at 37° C. Preferred antibodies of the invention are resistant to pepsin digestion at a pepsin concentration of 30 U/ml, when exposed to said pepsin for or for at least 15, 30, 45, 60, or 90 minutes at 37° C. Each of the exemplified antibodies, Net 14 and Net 83, display this property. For example, both Net 14 and Net 83 show resistance to pepsin when exposed to 30 U/ml pepsin for 1 hour, and Net 83 shows resistance to pepsin when exposed for 1.5 hours (90 minutes). Both antibodies also show resistance to pepsin when exposed to 107 U/ml pepsin for 15 or 30 minutes. As explained above, such resistance is observed at an acidic pH, for example at a pH of below 3.0 or 2.0, for example at a pH of 1.8 or 2.5.

Thus, such antibodies retain the ability to reduce the toxic (cytotoxic) activity of NetB after exposure to pepsin under these conditions, or other conditions as described herein. Alternatively, or additionally, such antibodies retain the ability to bind to NetB after exposure to pepsin under these conditions, or other conditions as described herein.

Thus, also preferred are antibodies (or binding proteins) which have detectable or measurable activity, e.g. binding to NetB or NetB inhibition, after exposure to pepsin, e.g. exposures to pepsin as described herein, e.g. exposure to 30 U/ml or at least 100 U/ml pepsin, e.g. 107 U/ml pepsin, for 15, 30, 45, 60, or 90 minutes, e.g. at 37° C. More preferably antibodies (or binding proteins) have significant NetB binding or inhibition activity after such exposure, e.g. an activity or binding which is not significantly reduced when compared to the activity observed when no pepsin exposure occurs.

In certain embodiments, after exposure to pepsin, binding proteins or antibodies of invention can still inhibit or reduce (or neutralise) the ability of NetB, e.g. NetB as produced by C. perfringens, to cause haemolysis of cells by at least 30%, at least 40%, at least 50%, at least 55%, at least 60%, at least 70%, at least 75%, at least 80%, at least 85%, at least 90%, at least 92%, at least 95% or at least 98%. Preferred antibodies of the invention have the ability to almost completely inhibit NetB toxicity, for example NetB induced haemolysis, after exposure to pepsin, for example at least 70%, 75%, 80%, 85%, 90% or 95% inhibition can be observed. Alternatively, at least 30%, 35%, 40%, 45%, 50%, 55%, 60%, or 65% inhibition can be observed.

Typically, such % inhibition (and other percentage inhibition levels as described herein) is in comparison with (or relative to) an appropriate control assay or control level, for example a control assay or control level in the absence of a binding protein or antibody (anti-NetB antibody, for example a negative control or background level or assay. Thus, a 0% inhibition (control) level (or conversely a 100% or maximum haemolysis level) is typically the level in the absence of a binding protein or antibody (anti-NetB antibody).

In addition, such % inhibition after exposure to pepsin (in the presence of pepsin), can also conveniently be compared to an equivalent % inhibition in the absence of pepsin in order to assess or determine the resistance or relative resistance of the antibody (or binding protein) to pepsin. Preferred antibodies (or binding proteins) of the invention maintain a significant or measurable ability to bind to NetB and/or to inhibit (or reduce or neutralise) the toxic activity of NetB as described elsewhere herein, after exposure to pepsin. For example, the ability of such antibodies (or binding proteins) to bind to NetB and/or to inhibit (or reduce or neutralise) the toxic activity of NetB is not significantly reduced by exposure to pepsin.

In certain embodiments, following exposure to pepsin, antibodies (or binding proteins) of the present invention have an IC₅₀ (e.g. for the inhibition, reduction, or neutralisation of NetB, for example inhibition of NetB haemolysis of target cells, e.g. red blood cells, preferably chicken red blood cells) of 400 nM or less, 350 nM or less, 300 nM or less, 280 nM or less, 260 nM or less, 250 nM or less, 240 nM or less, 220 nM or less, 210 nM or less, 200 nM or less, 190 nM or less, 180 nM or less, 170 nM or less, 160 nM or less, 150 nM or less, 140 nM or less, 130 nM or less, 120 nM or less, 110 nM or less, 100 nM or less, 90 nM or less, 80 nM or less, 70 nM or less, 60 nM or less, 50 nM or less, 40 nM or less, 35 nM or less, 30 nM or less, 25 nM or less, or 20 nM or less. In some embodiments, the IC₅₀ is 20 or 30 to 300, 250 or 200 nM; or 20 or 30 to 160 nM; or 20 or 30 to 150 nM; or 20 or 30 to 120 nM; or 20 or 30 to 110 nM. Preferably, following exposure to pepsin, antibodies (or binding proteins) of the invention have an IC₅₀ of 400 nM or less, 350 nM or less, 300 nM or less, 260 nM or less, 240 nM or less, 220 nM or less, 210 nM or less, 200 nM or less, 190 nM or less, 180 nM or less, 170 nM or less, 160 nM or less, 150 nM or less, 140 nM or less, 130 nM or less, 120 nM or less. Particular exemplary IC₅₀ values and assay conditions are described elsewhere herein or are shown in the Examples. For example, the exemplified antibody Net 14 still shows an IC₅₀ of 113 nM and Net 83 still shows an IC₅₀ of 208 nM after exposure to 30 U/ml pepsin for 30 minutes at 37° C. Thus, in some embodiments antibodies (or binding proteins) of the invention have an IC₅₀ of 220 nM or less after exposure to 30 U/ml pepsin for 30 minutes at 37° C. In such embodiments an exemplary pH is 1.8 or 2.5. In such embodiments an exemplary concentration of NetB is an EC₈₀ concentration or dose, or a concentration of 20 nM. Other exemplary IC₅₀ values are described elsewhere herein.

Appropriate assays to assess the ability to bind to NetB and/or to neutralise (or inhibit or reduce) the toxic activity of NetB are well known in the art and any of these may be used. Preferred and exemplary assays are described elsewhere herein, for example haemolytic assays, or ELISA or SPR binding assays.

Thus, the preferred IC₅₀ values as described above are preferably as determined in an appropriate toxicity assay (NetB inhibition assay) carried out under appropriate conditions to enable IC₅₀ values to be measured or determined as described elsewhere herein. Thus, the exemplary values provided above can for example be values as determined when the antibodies, preferably VHH antibodies (or binding proteins) of the invention are exposed to 30 U/ml or 107 U/ml pepsin for or for at least 15, 30, 45, 60, or 90 minutes at 37° C., before being assessed in a toxicity assay (NetB inhibition assay) using an appropriate concentration (toxic concentration) of NetB (e.g. an EC₈₀ concentration or dose), preferably a haemolytic assay, more preferably a haemolytic assay where lysis of chicken red blood cells is assessed, for example using a NetB concentration of 20 nM. Exemplary pHs are pH 1.8 or pH 2.5. Thus, the exemplary values provided above can for example be values as determined when NetB is used at an EC₈₀ concentration (e.g. a concentration of 20 nM) and/or when the assay is carried out at a pH of 1.8 or 2.5.

Preferred antibodies (or binding proteins) of the invention retain at least 50%, e.g. at least 55%, 60%, 65%, 70%, 75%, 80% or 85% ability to inhibit or neutralise NetB, when exposed to pepsin (or in the presence of pepsin), in comparison with the same antibody (or binding protein) which has not been exposed to pepsin (or in the absence of pepsin). Again, such values can be values as determined when the antibodies, preferably VHH antibodies (or binding proteins) of the invention are exposed to 30 U/ml or 107 U/ml pepsin for or for at least 15, 30, 45, 60, or 90 minutes at 37° C., before being assessed in a toxicity assay (NetB inhibition assay) using an appropriate concentration (toxic concentration) of NetB, preferably a haemolytic assay, more preferably a haemolytic assay where lysis of chicken red blood cells is assessed for example using a NetB concentration of 20 nM. Exemplary pHs are pH 1.8 or pH 2.5. Particular exemplary % retention values and assay conditions are also shown in the Examples. For example, the exemplified antibody Net 14 retains 84% activity after 30 minutes exposure to 30 U/ml pepsin at pH 1.8, and 45% after 45 or 60 minutes exposure, whereas Net 83 retains 72% activity after 30 minutes exposure to 30 U/ml pepsin, and 35% after 45, 60 or 90 minutes exposure. Thus, in some embodiments, antibodies (or binding proteins) with these, or at least these, % retention values, under these conditions are preferred.

In some embodiments, antibodies (or binding proteins) of the invention are resistant to pancreatin or resistant to pancreatin digestion. The term “resistant to pancreatin” or “resistant to pancreatin digestion” as used herein includes that the antibodies (or binding proteins) do not lose, or maintain, a significant or measurable ability to bind to NetB and/or to inhibit (or reduce or neutralise) the toxic activity of NetB as described elsewhere herein, after exposure to or contact with pancreatin. For example, antibodies with this property are capable of inhibiting (or reducing or neutralising) the ability of NetB to cause haemolysis of cells, for example appropriate target cells such as red blood cells, after contact with (or exposure to) pancreatin. Such antibodies are also capable of binding to NetB, for example NetB as produced by C. perfringens, after contact with (or exposure to) pancreatin. Viewed alternatively, such antibodies retain activity to inhibit (or reduce or neutralise) NetB after coming into contact with (or exposure to) pancreatin. These abilities of the antibodies after exposure to pancreatin (or in the presence of pancreatin) can conveniently be compared to the level observed in the absence of pancreatin, or without exposure to pancreatin.

Any appropriate level or concentration of pancreatin can be used. Preferred and exemplary levels would reflect those as found in the small intestine or GI tract. An exemplary level disclosed herein is a concentration of 0.5 mg/ml, as this correlates to >100 U/ml protease activity and is a commonly used value for simulating intestinal fluid/intestinal digestion. A concentration of 1 mg/ml or 0.1 mg/ml can also be used. Other exemplary exposure conditions would be a temperature of 37° C. for an appropriate time period, e.g. for or for at least 15, 30, 45, 60, 90 or 120 minutes and for example up to 2.5 hours, up to 2 hours, up to 1.5 hours, or up to one hour. An exemplary pH would be a pH of at or around pH 6.8.

Thus, preferred antibodies (or binding proteins) of the invention are resistant to pancreatin digestion at a pancreatin concentration of 0.5 mg/ml, e.g. at a temperature of 37° C. Preferred antibodies (or binding proteins) of the invention are resistant to pancreatin digestion at a pancreatin concentration of 0.5 mg/ml or 1 mg/ml, when exposed to said pancreatin for or for at least 15, 30, 45, 60, 90 or 120 minutes at 37° C. For example, Net 83 shows resistance to pancreatin when exposed to 0.5 mg/ml pancreatin for 15, 30, 45, 60, or 90 minutes, with some resistance still being shown even at 120 minutes. Net 83 also shows resistance to pancreatin when exposed to 1 mg/ml pancreatin for 30 minutes. Such resistance can conveniently be observed at pH of at or around pH 6.8.

Such antibodies preferably retain the ability to reduce the toxic (cytotoxic) activity of NetB after exposure to pancreatin under these conditions, or other conditions as described herein. Alternatively, or additionally, such antibodies retain the ability to bind to NetB after exposure to pancreatin under these conditions, or other conditions as described herein.

Appropriate assays to assess the ability to bind to NetB and/or to neutralise (or inhibit or reduce) the toxic activity of NetB are well known in the art and any of these may be used. Preferred and exemplary assays are described elsewhere herein, for example haemolytic assays, or ELISA or SPR binding assays.

Although haemolysis based NetB inhibition assays can conveniently be used for assessing activity of the antibodies of the invention after exposure to proteases, protease resistance can also conveniently be assessed on a gel, e.g. an SDS-PAGE gel, and carrying out an assessment of whether the antibody protein has been significantly or completely digested, or whether non-digested antibody protein is still present. Such assessment can conveniently be carried out by detecting bands on a gel at the appropriate molecular weights.

In some embodiments the antibodies (or binding proteins) of the invention advantageously exhibit the further property of showing stability at low pHs, for example at a pH of below 3.0 or below 2.0, e.g. at a pH of 1.8 or 2.5.

Thus, preferred antibodies of the invention are also stable, e.g. are not degraded or destroyed, or retain their functional activity, e.g. one or more of the functional properties as described herein such as the ability to bind NetB, or the ability to reduce the toxic (cytotoxic) activity of NetB, or other functional properties, after they have been exposed to (or contacted with or incubated at) an acidic pH, for example a pH of below 3.0 or below 2.0, e.g. a pH of 1.8 or 2.5.

Thus, such antibodies retain the ability to reduce the toxic (cytotoxic) activity of NetB after exposure to such low pHs. Alternatively, or additionally, such antibodies retain the ability to bind to NetB after exposure to such low pHs.

Appropriate assays to assess the retention of these functional activities after the antibodies (or binding proteins) have been exposed to low pH are described elsewhere herein. Exemplary exposure, contact or incubation times to the low pHs as outlined above might be exposure, etc., for 15, 30, 45, 60, 90, or 120 minutes, or at least 15, 30, 45, 60, 90, or 120 minutes, or up to 15, 30, 45, 60, 90, or 120 minutes. An exemplary temperature might be 37° C.

In some embodiments the antibodies (or binding proteins) of the invention advantageously exhibit the further property of showing stability in water, in particular tap water. Such a property is particularly advantageous in some animal health fields, e.g. where it is desired to treat relatively large numbers of animals, as it opens up the possibility to administer or deliver the active agent in the drinking water or as a feed component provided to the animals. Thus, such a property is highly desirable for the use of the antibodies of the invention to treat avian animals for NetB associated diseases such as NE.

In some embodiments, antibodies (or binding proteins) of the invention have a stability in water, in particular tap water, of at least 4 or 7 days, for example at least or up to 14, 21, or 28 days, or at least or up to 2 months or 3 months. These time points can refer to a stability at room temperature.

Preferably, the above described abilities and properties are observed at a measurable or significant level and more preferably at a statistically significant level, when compared to appropriate control levels. Appropriate significance levels are discussed elsewhere herein. More preferably, one or more of the above described abilities and properties are observed at a level which is measurably better, or more preferably significantly better (preferably statistically significantly better), when compared to the abilities observed for prior art antibodies.

In any statistical analysis referred to herein, preferably the statistically significant difference over a relevant control or other comparative entity or measurement has a probability value of ≤0.1 or <0.1, preferably s 0.05 or <0.05. Appropriate methods of determining statistical significance are well known and documented in the art and any of these may be used.

In some embodiments, binding proteins or antibodies of the present invention have one or more, preferably two or more, or three or more, or four or more, or five or more, most preferably all, of the functional properties, in particular the preferred functional properties, described herein. Examples of preferred functional properties and further details regarding said properties are described elsewhere herein, and include i) a high affinity for NetB, e.g. when measured by Biacore (the ability to bind NetB is observed at pH 6.0, as well as pH 7.4), ii) the ability to reduce the toxic activity of NetB (this ability is observed at pH 6.0, as well as pH 7.4, and preferably at a temperature of both 42° C. and 37° C.), resistance to various proteases, for example, iii) resistance to pepsin digestion (e.g. at a pepsin concentration of 30 U/ml and a temperature of 37° C.), iv) resistance to chymotrypsin digestion (e.g. at a chymotrypsin concentration of 1 μg/ml and a temperature of 37° C.), and optionally v) resistance to pancreatin digestion (e.g. at a pancreatin concentration of 0.5 mg/ml and a temperature of 37° C.). The preferred antibodies (or binding proteins) also show stability in water.

As used throughout the entire application, the terms “a” and “an” are used in the sense that they mean “at least one”, “at least a first”, “one or more” or “a plurality” of the referenced components or steps, except in instances wherein an upper limit is thereafter specifically stated. Therefore, an “antibody”, as used herein, means “at least a first antibody”.

In addition, where the terms “comprise”, “comprises”, “has” or “having”, or other equivalent terms are used herein, then in some more specific embodiments, for example in the definition of the CDR or FR sequences herein, these terms include the term “consists of” or “consists essentially of”, or other equivalent terms.

Nucleic acid molecules (e.g. one or more nucleic acid molecules) comprising nucleotide sequences that encode the binding proteins or antibodies of the present invention as defined herein or parts or fragments thereof, or nucleic acid molecules substantially homologous thereto, form yet further aspects of the invention.

Preferred nucleic acid molecules are those encoding a VHH antibody or a VH region or domain of the present invention (e.g., those encoding SEQ ID NO:1 or 9). Other preferred nucleic acid molecules are those encoding the sets of three CDR sequences as defined in any one of Tables A or B. Preferred such nucleic acid molecules also encode appropriate framework regions, e.g. FR1, FR2, FR3 and FR4 regions, preferably the sets of FR sequences as defined in any one of Tables A or B.

The term “substantially homologous” as used herein in connection with an amino acid or nucleic acid sequence includes sequences having at least 60%, 65%, 70% or 75%, preferably at least 80%, and even more preferably at least 85%, 86%, 87%, 88%, 89%, 90%, 91%, 92%, 93%, 94%, 95%, 96%, 97%, 98% or 99%, sequence identity to the amino acid or nucleic acid sequence disclosed.

Substantially homologous sequences of the invention thus include single or multiple base or amino acid alterations (additions, substitutions, insertions or deletions) to the sequences of the invention. At the amino acid level preferred substantially homologous sequences contain up to 5, e.g. only 1, 2, 3, 4 or 5, preferably 1, 2, 3 or 4, preferably 1, 2 or 3, more preferably 1 or 2, altered amino acids, in one or more of the framework regions and/or one or more of the CDRs making up the sequences of the invention. In addition, at the amino acid level, preferred substantially homologous sequences contain up to 5, e.g. only 1, 2, 3, 4 or 5, preferably 1, 2, 3 or 4, preferably 1, 2 or 3, more preferably 1 or 2, altered amino acids, in the combined framework regions (e.g. the four framework regions), and/or the combined CDRs (e.g. the three CDR regions) making up the sequences of the invention. In addition, at the amino acid level, preferred substantially homologous sequences contain up to 5, e.g. only 1, 2, 3, 4 or 5, preferably 1, 2, 3 or 4, preferably 1, 2 or 3, more preferably 1 or 2, altered amino acids, in one or more of the VHH domains of the invention. Said alterations can be with conservative or non-conservative amino acids, or a mixture thereof. Preferably said alterations are substitutions, preferably conservative amino acid substitutions.

In certain embodiments, if a given starting sequence is relatively short (e.g. five amino acids in length), then fewer amino acid substitutions may be present in sequences substantially homologous thereto as compared with the number of amino acid substitutions that might optionally be made in a sequence substantially homologous to a longer starting sequence. For example, in certain embodiments, a sequence substantially homologous to a starting VH CDR1 sequence in accordance with the present invention, e.g. a starting VH CDR1 sequence which in some embodiments may be five amino acid residues in length, preferably has 1 or 2 (more preferably 1) altered amino acids in comparison with the starting sequence. Accordingly, in some embodiments the number of altered amino acids in substantially homologous sequences (e.g. in substantially homologous CDR sequences) can be tailored to the length of a given starting CDR sequence. For example, different numbers of altered amino acids can be present depending on the length of a given starting CDR sequence such as to achieve a particular % sequence identity in the CDRs, for example a sequence identity of at least 60%, 70%, 75%, 80%, 85%, 86%, 87%, 88%, 89%, 90%, 91%, 92%, 93%, 94%, 95%, 96%, 97%, 98% or 99%.

Routine methods in the art such as alanine scanning mutagenesis and/or deep mutational scanning (which aims to perform all possible mono-substitutions on all selected residues within a given protein sequence) and/or analysis of crystal structure of the antigen-antibody complex can be used in order to determine which amino acid residues of the CDRs do not contribute or do not contribute significantly to antigen binding and therefore are good candidates for alteration or substitution in the embodiments of the invention involving substantially homologous sequences.

Once identified, the addition, deletion, substitution or insertion of one or more amino acids in the amino acid sequence of a parent antibody to form a new antibody, wherein said parent antibody is one of the antibodies of the invention as defined elsewhere herein, and testing the resulting new antibody to identify antibodies that bind to NetB in accordance with the invention can be carried out using techniques which are routine in the art. Such methods can be used to form multiple new antibodies that can all be tested for their ability to bind NetB. Preferably said addition, deletion, substitution or insertion of one or more amino acids takes place in one or more of the CDR domains.

For example, said manipulations could conveniently be carried out by genetic engineering at the nucleic acid level wherein nucleic acid molecules encoding appropriate binding proteins and domains thereof are modified such that the amino acid sequence of the resulting expressed protein is in turn modified in the appropriate way. Testing the ability of one or more of the modified antibodies/binding proteins to bind to NetB can be carried out by any appropriate method, which are well known and described in the art. Suitable methods are also described elsewhere herein and in the Examples section.

New antibodies produced, obtained or obtainable by these methods form a yet further aspect of the invention.

The term “substantially homologous” also includes modifications or chemical equivalents of the amino acid and nucleotide sequences of the present invention that perform substantially the same function as the proteins or nucleic acid molecules of the invention in substantially the same way. For example, any substantially homologous antibody should retain the ability to bind to NetB as described above. Preferably, any substantially homologous antibody should retain one or more (or all) of the functional capabilities of the starting antibody.

Substantially homologous sequences of proteins of the invention include, without limitation, conservative amino acid substitutions, or for example alterations that do not affect the VH, VL or CDR domains of the antibodies, e.g. antibodies where tag sequences, toxins or other components are added that do not contribute to the binding of antigen, or alterations to convert one type or format of binding protein, antibody molecule or fragment to another type or format of binding protein, antibody molecule or fragment (e.g. conversion from VHH to Fab or scFv or whole antibody, e.g. a full length heavy chain only antibody, or vice versa), or the conversion of an antibody molecule to a particular class or subclass of antibody molecule (e.g. the conversion of an antibody molecule to IgG or a subclass thereof, e.g. IgG₂ or IgG₃, for example to a camelid antibody, or to an IgA or IgY class of antibody), or the preparation of an Fc fusion, e.g. a VHH-Fc fusion.

A “conservative amino acid substitution”, as used herein, is one in which the amino acid residue is replaced with another amino acid residue having a similar side chain. Families of amino acid residues having similar side chains have been defined in the art, including basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. glycine, cysteine, alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). In other examples, families of amino acid residues can be grouped based on hydrophobic side groups or hydrophilic side groups.

Homology or sequence identity may be assessed by any convenient method. However, for determining the degree of homology or identity between sequences, computer programs that make multiple alignments of sequences are useful, for instance Clustal W (Thompson, Higgins, Gibson, Nucleic Acids Res., 22:4673-4680, 1994). If desired, the Clustal W algorithm can be used together with BLOSUM 62 scoring matrix (Henikoff and Henikoff, Proc. Natl. Acad. Sci. USA, 89:10915-10919, 1992) and a gap opening penalty of 10 and gap extension penalty of 0.1, so that the highest order match is obtained between two sequences wherein at least 50% of the total length of one of the sequences is involved in the alignment. Other methods that may be used to align sequences are the alignment method of Needleman and Wunsch (Needleman and Wunsch, J. Mol. Biol., 48:443, 1970) as revised by Smith and Waterman (Smith and Waterman, Adv. Appl. Math., 2:482, 1981) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton, SIAM J. Applied Math., 48:1073, 1988) and those described in Computational Molecular Biology, Lesk, e.d. Oxford University Press, New York, 1988, Biocomputing: Informatics and Genomics Projects.

Generally, computer programs will be employed for such calculations. Programs that compare and align pairs of sequences, like ALIGN (Myers and Miller, CAB/OS, 4:11-17, 1988), FASTA (Pearson and Lipman, Proc. Natl. Acad. Sci. USA, 85:2444-2448, 1988; Pearson, Methods in Enzymology, 183:63-98, 1990) and gapped BLAST (Altschul et al., Nucleic Acids Res., 25:3389-3402, 1997), BLASTP, BLASTN, or GCG (Devereux, Haeberli, Smithies, Nucleic Acids Res., 12:387, 1984) are also useful for this purpose. Furthermore, the Dali server at the European Bioinformatics institute offers structure-based alignments of protein sequences (Holm, Trends in Biochemical Sciences, 20:478-480, 1995; Holm, J. Mol. Biol., 233:123-38, 1993; Holm, Nucleic Acid Res., 26:316-9, 1998).

By way of providing a reference point, sequences according to the present invention having at least 60%, 65%, 70%, 75%, 80%, 85%, 86%, 87%, 88%, 89%, 90%, 91%, 92%, 93%, 94%, 95%, 96%, 97%, 98% or 99% homology, sequence identity etc. may be determined using the ALIGN program with default parameters (for instance available on Internet at the GENESTREAM network server, IGH, Montpellier, France).

Preferably, any substantially homologous antibody should retain the ability to specifically bind to the same epitope of NetB as recognized by the starting antibody in question, for example, the same epitope recognized by the CDR domains of one or more of the antibodies of the invention or the VH (VHH) domains of the invention as described herein, e.g. bind to the same epitope as one or more of the various antibodies of the invention (e.g. one or more of the VHH antibodies Net 83 or Net 14, as shown in Tables A and B, respectively). Thus, preferably, any substantially homologous antibody should retain the ability to compete, in a suitable assay, with one or more of the various antibodies of the invention (e.g. one or more of the VHH antibodies Net 83 or Net 14, as shown in Tables A and B, respectively) for binding to NetB.

Binding to the same epitope can be readily tested by methods well known and described in the art, e.g. using binding assays, e.g. a competition assay, or by analysis of the crystal structure of the antigen-antibody complex. Thus, antibodies which bind to the same epitope as one or more of the various antibodies of the invention, for example as assessed or determined by a competition assay, analysis of the crystal structure of the antigen-antibody complex, or by mutational studies of individual residues (e.g. using alanine scanning and/or deep mutational scanning), form yet further aspects of the invention. Retention of other functional properties can also readily be tested by methods well known and described in the art or herein.

Thus, a person skilled in the art will appreciate that binding assays can be used to test whether any antibodies, for example “substantially homologous” antibodies, have the same binding specificities, e.g. bind to the same epitope, or with the same or equivalent affinity, as the antibodies and antibody fragments of the invention, for example, binding assays such as competition assays or ELISA assays as described elsewhere herein. SPR assays, such as BIAcore assays as described elsewhere herein, could also readily be used to establish whether antibodies, for example “substantially homologous” antibodies, can bind to NetB, and optionally the affinity, e.g. KD, of such binding. The skilled person will be aware of other suitable methods and variations.

As outlined below, a competition binding assay can be used to test whether antibodies, for example “substantially homologous” antibodies retain the ability to specifically bind to the same or substantially the same epitope of NetB as recognized by one or more of the antibodies of the invention as shown in the various sequence Tables herein, or have the ability to compete with one or more of the various antibodies of the invention as shown in the various sequence Tables herein. The method described below is only one example of a suitable competition assay. The skilled person will be aware of other suitable methods and variations.

An exemplary competition assay involves assessing the binding of various effective concentrations of an antibody of the invention to NetB in the presence of varying concentrations of a test antibody (e.g. a substantially homologous antibody). The amount of inhibition of binding induced by the test antibody can then be assessed. A test antibody that shows increased competition with an antibody of the invention at increasing concentrations (i.e. increasing concentrations of the test antibody result in a corresponding reduction in the amount of antibody of the invention binding to NetB) is evidence of binding to the same or substantially the same epitope. Preferably, the test antibody significantly reduces the amount of antibody of the invention that binds to NetB. Preferably, the test antibody reduces the amount of antibody of the invention that binds to NetB by at least about 95%. ELISA or flow cytometry assays may be used for assessing inhibition of binding in such a competition assay but other suitable techniques would be well known to a person skilled in the art.

Such antibodies (monoclonal antibodies) which have the ability to specifically bind to substantially the same (or the same) epitope of NetB or an overlapping epitope of NetB as recognized by the antibodies of the invention (e.g. one or more of the VHH antibodies Net 83 or Net 14, as shown in Tables A and B, respectively) or which have the ability to compete with one or more of the various antibodies of the invention (e.g. one or more of the VHH antibodies Net 83 or Net 14, as shown in Tables A and B, respectively) are further embodiments of the present invention.

The term “competing antibodies”, as used herein, refers to antibodies that bind to about, substantially or essentially the same, or even the same, epitope as a “reference antibody”. “Competing antibodies” include antibodies with overlapping epitope specificities. Competing antibodies are thus able to effectively compete with a reference antibody for binding to NetB. Preferably, the competing antibody can bind to the same epitope as the reference antibody. Alternatively viewed, the competing antibody preferably has the same epitope specificity as the reference antibody.

“Reference antibodies” as used herein are antibodies which can bind to NetB in accordance with the invention which preferably have a VH domain as defined herein, more preferably have a VH domain or are a VHH antibody comprising SEQ ID NO: 1 or 9 (or the relevant three CDR sequences of said sequences) as outlined in Tables A or B.

The identification of one or more competing antibodies or antibodies that bind to the same epitope is a straightforward technical matter now that reference antibodies such as those outlined in the sequence Tables herein have been provided. As the identification of competing antibodies or antibodies that bind to the same epitope can be determined in comparison to a reference antibody, it will be understood that actually determining the epitope to which either or both antibodies bind is not in any way required in order to identify a competing antibody or an antibody that binds to the same epitope. However, epitope mapping can be performed using standard techniques, if desired, some of which are outlined elsewhere herein.

The terms “antibody” and “immunoglobulin”, as used herein, refer broadly to any immunological binding agent that comprises an antigen binding domain, including polyclonal and monoclonal antibodies. Monoclonal antibodies are however preferred. In other words, in some embodiments antibodies of the invention are not polyclonal antibodies. Depending on the type of constant domain in the heavy chains, whole antibodies are assigned to one of five major classes: IgA, IgD, IgE, IgG, and IgM and the antibodies of the invention may be in any one of these classes, or may be chicken IgY antibodies. In some embodiments, IgY antibodies are not used. Several of these are further divided into subclasses or isotypes, such as IgG1, IgG2, IgG3, IgG4, and the like, for example camelid antibodies are IgG antibodies which often have IgG2 or IgG3 constant domains. The heavy-chain constant domains that correspond to the difference classes of immunoglobulins are termed a, 6, s, y and π, respectively. The subunit structures and three-dimensional configurations of different classes of immunoglobulins are well known.

Generally, where whole antibodies rather than antigen binding regions are used in the invention, IgG are preferred because they are the most common antibodies in the physiological situation and because they are most easily made in a laboratory setting.

The “light chains” of mammalian antibodies are assigned to one of two clearly distinct types: kappa (κ) and lambda (λ), based on the amino acid sequences of their constant domains and some amino acids in the framework regions of their variable domains.

The term “heavy chain complementarity determining region” (“heavy chain CDR”) as used herein refers to regions of hypervariability within the heavy chain variable region (V_{H} domain) of an antibody molecule or within a VHH antibody molecule. The heavy chain variable region has three CDRs termed heavy chain CDR1, heavy chain CDR2 and heavy chain CDR3 from the amino terminus to carboxy terminus. The heavy chain variable region also has four framework regions (FR1, FR2, FR3 and FR4 from the amino terminus to carboxy terminus). These framework regions separate the CDRs.

The term “heavy chain variable region” (V_{H} domain) as used herein refers to the variable region of a heavy chain of an antibody molecule.

The term “light chain complementarity determining region” (“light chain CDR”) as used herein refers to regions of hypervariability within the light chain variable region (V_{L} domain) of an antibody molecule. Light chain variable regions have three CDRs termed light chain CDR1, light chain CDR2 and light chain CDR3 from the amino terminus to the carboxy terminus. The light chain variable region also has four framework regions (FR1, FR2, FR3 and FR4 from the amino terminus to carboxy terminus). These framework regions separate the CDRs.

The term “light chain variable region” (V_{L} domain) as used herein refers to the variable region of a light chain of an antibody molecule.

As will be understood by those in the art, the immunological binding agents encompassed by the term “antibody” includes or extends to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)₂, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like.

The techniques for preparing and using various antibody-based constructs and fragments are well known in the art.

Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

In all embodiments of the invention, single domain antibodies (also referred to as VHH antibodies, sdAbs, DABs, dAbs, nanobodies, camelid antibodies, vNAR (shark) antibodies, VH antibodies or VL antibodies) are preferred, in particular VHH antibodies, nanobodies, camelid antibodies, and vNAR (shark) antibodies. Such antibodies comprise a single monomeric variable antibody domain, usually a VH domain, which can bind to antigen (although single VL domains which have the ability to bind antigen have been described and can be used). Thus, in some such preferred embodiments the antibodies (or antigen binding domains) of the invention comprise (or consist of) one (or a single, or only a single) heavy chain variable region (VH or VHH), although in some embodiments a number of these individual heavy chain variable regions with the same or different sequences can be present together in the same construct or molecule. Thus, in some such embodiments, the antibodies (or antigen binding domains) of the invention, have only three CDRs (typically together with four FR regions in the standard or usual order). Sometimes such antibodies can be referred to as heavy chain only antibodies.

Such antibodies can be obtained or prepared using standard techniques which are well known and described in the art. For example, such antibodies can be obtained by immunizing appropriate animals, e.g. camelids such llamas, or sharks, with the desired antigen and then cloning the VH domains of the antibodies generated into appropriate expression vectors and selecting for binders. Libraries of VH domains (e.g. phage display libraries of human VH domains) are also available or can be generated and can then be screened.

In certain embodiments, the antibody or antibody fragment of the present invention comprises all or a portion of a heavy chain constant region, such as an IgG1, IgG2, IgG3, IgG4, IgA1, IgA2, IgE, IgM, IgD or IgY constant region. Preferably, the heavy chain constant region is an IgG, IgA or IgY heavy chain constant region or a portion thereof. Furthermore, the antibody or antibody fragment can comprise all or a portion of a kappa light chain constant region or a lambda light chain constant region, or a portion thereof. All or part of such constant regions may be produced naturally or may be wholly or partially synthetic. Appropriate sequences for such constant regions are well known and documented in the art. When a full complement of constant regions from the heavy and/or light chains are included in the antibodies of the invention, such antibodies are typically referred to herein as “full length” antibodies or “whole” antibodies.

In other embodiments it is preferred that no constant regions, e.g. no heavy chain or light chain constant regions, are present, e.g. a variable domain or heavy chain variable domain (VH) is the only part of an antibody that is present.

The antibodies or antibody fragments can be produced naturally or can be wholly or partially synthetically produced.

Many antibodies or antibody fragments comprise an antibody light chain variable region (V_{L}) that comprises three CDR domains and an antibody heavy chain variable region (V_{H}) that comprises three CDR domains. Said VL and VH generally form the antigen binding site.

However, it is well documented in the art that the presence of three CDRs from the light chain variable domain and three CDRs from the heavy chain variable domain of an antibody is not always necessary for antigen binding. Thus, constructs smaller than the above classical antibody fragment are known to be effective.

For example, camelid antibodies have an extensive antigen binding repertoire but are devoid of light chains. Also, results with single domain antibodies comprising VH domains alone or VL domains alone show that these domains can bind to antigen with acceptably high affinities and have other advantages such as their small size and ease of production. Thus, three CDRs can effectively bind antigen and such single domain antibodies (for example VHH antibodies, sdAbs, DABs, dAbs, nanobodies, camelid antibodies, vNAR (shark) antibodies, VH antibodies or VL antibodies, in particular VHH antibodies, nanobodies, camelid antibodies, and vNAR (shark) antibodies) are exemplified herein and this type of antibody is preferred (e.g. a VHH antibody).

The antibody, binding protein and nucleic acid molecules of the invention are generally “isolated” or “purified” molecules insofar as they are distinguished from any such components that may be present in situ within a human or animal body (e.g. a camelid) or a tissue sample derived from a human or animal body (e.g. a camelid). The sequences may, however, correspond to or be substantially homologous to sequences as found in a human or animal body (e.g. a camelid). Thus, the term “isolated” or “purified” as used herein in reference to nucleic acid molecules or sequences and proteins or polypeptides, e.g. antibodies, refers to such molecules when isolated from, purified from, or substantially free of their natural environment, e.g. isolated from or purified from the human or animal body (if indeed they occur naturally), or refers to such molecules when produced by a technical process, i.e. includes recombinant and synthetically produced molecules.

It can be noted that in embodiments the antibodies etc., of the invention do not occur in nature and are, in that respect, man-made constructs in that they do not correspond to molecules that occur naturally. For example, preferred antibodies are single domain antibodies which can be engineered or recombinantly produced, and even in species that produce such antibodies naturally, e.g. camelids, such species have been experimentally induced to do so, e.g. by immunization. In other words in embodiments the antibodies, etc., of the invention are non-native.

The term “fragment” as used herein refers to fragments of biological relevance, e.g. fragments that contribute to antigen binding, e.g. form part or all of the antigen binding site, and/or contribute to the functional properties of the NetB antibody. Certain preferred fragments comprise or consist of a heavy chain variable region (V_{H} domain/VHH domain or the three VH CDRs) of the antibodies of the invention.

A person skilled in the art will appreciate that the proteins and polypeptides of the invention, such as the heavy and light chain CDRs, the heavy and light chain variable regions, antibodies and antibody fragments, may be prepared in any of several ways well known and described in the art, but are most preferably prepared using recombinant methods.

Nucleic acid fragments encoding the heavy and/or light chain variable regions of the antibodies of the invention, as appropriate, can be derived or produced by any appropriate method, e.g. by cloning or synthesis.

Once nucleic acid fragments encoding the heavy and/or light chain variable regions of the antibodies of the invention have been obtained, these fragments can be further manipulated by standard recombinant DNA techniques, for example to convert the variable region fragments into full length antibody molecules with appropriate constant region domains, or into particular formats of antibody fragment discussed elsewhere herein, e.g. single domain antibodies such as VHH, Fab fragments, scFv fragments, etc. Typically, or as part of this further manipulation procedure, the nucleic acid fragments encoding the antibody molecules of the invention are generally incorporated into one or more appropriate expression vectors in order to facilitate production of the antibodies of the invention or for example to facilitate selection or screening, e.g. by incorporating into phage display vectors.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the invention and regulatory sequences selected on the basis of the host cells to be used for expression, which are operatively linked to the nucleic acid molecule. Operatively linked is intended to mean that the nucleic acid is linked to regulatory sequences in a manner that allows expression of the nucleic acid.

The invention therefore contemplates an expression vector, e.g. a recombinant expression vector, containing or comprising a nucleic acid molecule of the invention, or a fragment thereof, and the necessary regulatory sequences for the transcription and translation of the protein sequence encoded by the nucleic acid molecule of the invention.

Expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. Suitable methods for transforming and transfecting host cells can be found in Sambrook et al., 1989 (Sambrook, Fritsch and Maniatis, Molecular Cloning: A Laboratory Manual, 2nd Ed., Cold Spring Harbor Press, Cold Spring Harbor, NY, 1989) and other laboratory textbooks.

Suitable host cells include a wide variety of eukaryotic host cells and prokaryotic cells, or plant cell-based or fungal expression systems (e.g. Saccharomyces cerevisiae) can be used, as will be well known to a person skilled in the art. For example, the proteins of the invention may be expressed in yeast cells, such as Pichia or Saccharomyces, e.g. Saccharomyces cerevisiae, or mammalian cells. In addition, the proteins of the invention may be expressed in prokaryotic cells, such as Escherichia coli (E. coli). In some embodiments expression in Pichia or E. coli is preferred.

The proteins of the invention may also be prepared by chemical synthesis using techniques well known in the chemistry of proteins such as solid phase synthesis.

A yet further aspect provides an expression construct or expression vector or expression system (e.g. a viral or bacterial or other expression construct, vector or system), e.g. one or more expression constructs or expression vectors, comprising one or more of the nucleic acid fragments or segments or molecules of the invention. Preferably the expression constructs or vectors or systems are recombinant. Preferably said constructs or vectors or systems further comprise the necessary regulatory sequences for the transcription and translation of the protein sequence encoded by the nucleic acid molecule of the invention. Preferred constructs etc., are those which allow prolonged or sustained expression of the antibodies (or binding proteins) of the invention within the host target species, e.g. within avian species. Such expression can be transient, e.g. episomal, or more permanent, e.g. via genomic integration, providing sufficient levels and length of expression are achieved in order for a therapeutic or biological effect to be observed.

A yet further aspect provides a host cell (e.g. a mammalian or bacterial or yeast host cell) or virus, e.g. one or more host cells or viruses, comprising one or more expression constructs or expression vectors of the invention. Also provided are host cells (e.g. a mammalian or bacterial or yeast host cell) or viruses, e.g. one or more host cells or viruses, comprising one or more of the nucleic acid molecules of the invention. A host cell (e.g. a mammalian host cell or bacterial host cell, or yeast host cell) or virus expressing an antibody (or binding protein) of the invention forms a yet further aspect.

Such expression constructs or vectors or systems, or host cells or viruses, or other nucleic acid products or fragments encoding the antibodies (or binding proteins) of the invention can be administered as therapeutic agents to a subject to allow the production of the antibodies (or binding proteins) of the invention in situ within the subject and thereby exert their therapeutic effects. Such host cells can also be referred to as live delivery vectors.

Thus, as described elsewhere herein, preferred host cells are bacterial cells or strains, e.g. probiotic bacterial cells or strains, which can occur naturally or survive in the GI tract or small intestine, and are suitable for administration to an animal, e.g. for therapeutic administration. Examples of appropriate bacteria are known in the art and include Bacillus, e.g. Bacillus subtilis, Lactobacillus, such as Lactobacillus reuteri, Lactococcus, Salmonella, Escherichia coli and Listeria, in particular probiotic strains such as Bacillus, e.g. Bacillus subtilis, Lactobacillus, such as Lactobacillus reuteri. Yeast cells, such as Pichia (Pichia pistoris) or Saccharomyces (S. cerevisiae) can also be used. Such host cells are modified using appropriate methods to express the antibodies of the invention.

A yet further aspect of the invention provides a method of producing (or manufacturing) an antibody (or binding protein) of the present invention comprising a step of culturing the host cells of the invention. Preferred methods comprise the steps of (i) culturing a host cell comprising one or more of the expression vectors or one or more of the nucleic acid sequences of the invention under conditions suitable for the expression of the encoded antibody or binding protein; and optionally (ii) isolating or obtaining the antibody or binding protein from the host cell or from the growth medium/supernatant. Such methods of production (or manufacture) may also comprise a step of purification of the antibody or protein product and/or formulating the antibody or product into a composition, e.g. a pharmaceutical composition, including at least one additional component, such as a pharmaceutically acceptable carrier or excipient.

In embodiments when the antibody or protein of the invention is made up of more than one polypeptide chain (e.g. certain fragments such as Fab fragments or whole antibodies), then all the polypeptides are preferably expressed in the host cell, either from the same or a different expression vector, so that the complete proteins, e.g. antibody proteins of the invention, can assemble in the host cell and be isolated or purified therefrom.

In another aspect, the invention provides a method of binding NetB, comprising contacting a composition comprising NetB with an antibody or binding protein of the invention.

In yet another aspect, the invention provides a method of detecting NetB, comprising contacting a composition suspected of containing NetB with an antibody of the invention, under conditions effective to allow the formation of NetB/antibody complexes and detecting the complexes so formed.

Compositions comprising at least a first antibody (or binding protein) of the invention, or at least a first nucleic acid molecule or expression vector of the invention, or at least a first host cell of the invention, constitute further aspects of the present invention. Formulations (compositions) comprising one or more antibodies, etc., of the invention, optionally in a mixture with a suitable diluent, carrier or excipient constitute a preferred embodiment of the present invention. Such formulations may be for pharmaceutical use, e.g. for use in animal health applications or veterinary use, e.g. in farming, and thus compositions of the invention are preferably pharmaceutically acceptable or acceptable for administration to human or non-human animals, in particular avian species or birds, for example poultry. Suitable diluents, excipients and carriers are known to the skilled man. In some embodiments, said compositions may include sodium bicarbonate (or sodium hydrogen carbonate, NaHCO3), for example at a concentration of at or around 0.5 g/l.

The compositions according to the invention may be presented, for example, in a form suitable for oral, nasal, parenteral, intravenous, topical or rectal administration, or for mucosal delivery, and any of these modes of administration, or indeed any other appropriate mode of administration, can be used. A preferred mode of administration is oral administration and thus forms suitable for such oral administration are also preferred.

The active compounds (e.g. the antibodies of the invention) as defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms.

Injection solutions may, for example, be produced in the conventional manner, such as by the addition of preservation agents, such as p-hydroxybenzoates, or stabilizers, such as EDTA. The solutions may then be filled into injection vials or ampoules.

Oral administration is extremely convenient. However, as described elsewhere herein the antibodies (or binding proteins) of the present invention are advantageously highly stable, including at low pH, e.g. at a pH of below 3.0 or below 2.0, e.g. at pH 1.8, and resistant to various enzymes and proteases which means that oral administration is practically feasible. Importantly, direct oral administration, e.g. without coating or other means of protection is also practically feasible. Thus, antibodies (or binding proteins) of the invention can for example be administered via drinking water, or by adding the antibodies (or binding proteins) to appropriate animal feed, e.g. by incorporation into feed pellets or similar, or by using as a top dressing/sprinkling onto feed, all of which conveniently allow regular administration, e.g. daily or more often if desired. Such antibodies can also be administered by spraying the subjects to be treated, or by in ovo administration, or via the respiratory system, e.g. by inhalation.

Thus a yet further aspect of the invention provides food products or food additives or food supplements, for example an animal feed product or additive or supplement, comprising an antibody (or binding protein) of the invention or a host cell of the invention. Such feed products may take the form of a feed premix or a top dressing powder (e.g. for sprinkling onto feed) product. Animal feed products where an antibody (or binding protein) of the invention is mixed with or incorporated into animal feed are also provided.

Alternative means of oral delivery are described in the art. A preferred method can involve the use of live delivery systems and vectors such as microbial vectors. Microbial vectors include bacteria such as Bacillus, e.g. Bacillus subtilis, Lactobacillus, such as Lactobacillus reuteri, Lactococcus, Salmonella, Escherichia coli and Listeria. Yeast cells, such as Pichia or Saccharomyces can also be used. These microbial vectors or live vectors are engineered to express, and preferably secrete, the antibodies (or binding proteins) of the invention and deliver them, preferably directly, to the site of action, e.g. by oral delivery or mucosal delivery. Such microbial vectors are thus sometimes referred to as direct fed microbials (DFMs). Thus, such vectors, e.g. recombinant vectors, enable in situ delivery of the biotherapeutics as the antibodies (or binding proteins) can be secreted or otherwise produced by the microbial vectors within the animal. Delivery using such vectors advantageously protects the antibodies from the harsh GI environment until they are secreted, but also can maximise effectiveness and minimise off-target effects. Such vectors also allow for sustained and continuous production of the antibodies (or binding proteins) within the GI tract, and preferably show persistence in the GI tract through colonization, as per the parent (unmodified) strains.

Such vectors can also be produced relatively easily (and cost effectively) on a large scale and high doses can generally be administered without adverse effects, for example administration of a dose of at least 108 CFUs is possible, and this can be done once or twice daily if required. Preferred microbial vectors are thus engineered or recombinant bacterial strains, e.g. probiotic strains such as Bacillus, e.g. Bacillus subtilis, and Lactobacillus, such as Lactobacillus reuteri, both of which have been described as useful for oral administration of biomolecules, including antibodies/VHH antibodies, e.g. by secretion (see for example Del Rio, B., et al., 2019, Frontiers in Microbiology, 9, 3179). In some embodiments a preferred vector is Bacillus subtilis.

Suitable dosage units can be determined by a person skilled in the art.

The pharmaceutical compositions may additionally comprise further active ingredients/active agents (e.g. as described elsewhere herein) in the context of co-administration regimens or combined regimens.

A further aspect of the present invention provides the anti-NetB antibodies (or binding proteins) defined herein, or the nucleic acid molecules, expression vectors or host cells or compositions as defined herein, for use in therapy, in particular for use in the treatment or prevention of any disease or condition associated with (or characterised by) NetB or where NetB has a role, for example a causative (e.g. a wholly or partially causative role) or an essential role. For example, the anti-NetB antibodies (or binding proteins), etc., of the invention can be used in the treatment or prevention of any infection caused by a bacteria or other pathogen, wherein said infection is associated with NetB, or where NetB has a role, for example a causative (e.g. a wholly or partially causative role), or an essential role. Put another way, in accordance with the present invention the anti-NetB antibodies (or binding proteins), etc., may target and inhibit or reduce the function of NetB (e.g. the toxic function of NetB), in particular NetB as produced by C. perfringens, or other bacteria or pathogens. Thus, the anti-NetB antibodies (or binding proteins), etc., defined herein may be used in the treatment or prevention of any disease or condition where inhibition of NetB or blockade or reduction of NetB function is useful.

In other embodiments the present invention provides antibodies (or binding proteins) which have the ability to reduce or inhibit NetB function, and therefore can be used to treat or prevent NE, for example NE caused by or associated with C. perfringens.

Preferred embodiments provide the anti-NetB antibodies (or binding proteins) of the invention, or the nucleic acid molecules, expression vectors or host cells or compositions of the invention, for use in the treatment or prevention of bacterial infections in avian species such as poultry, for example bacterial infections of the GI tract, preferably C. perfringens infection. Particularly preferred is the treatment or prevention of Necrotic enteritis (NE), for example NE caused by or associated with NetB production, in particular NetB production by C. perfringens.

The administration of the binding proteins or antibodies or the nucleic acid molecules, expression vectors or host cells, or compositions, in the therapeutic methods and uses of the invention is carried out in pharmaceutically, therapeutically, or physiologically effective amounts, to subjects (animals, e.g. mammals or avian species, e.g. poultry) in need of treatment. Thus, said methods and uses may involve the additional step of identifying a subject (or subjects/group of subjects) in need of treatment. Appropriate and effective concentrations/doses to be administered can readily be determined by a person skilled in the art.

Treatment of diseases or conditions in accordance with the present invention (for example treatment of pre-existing disease) includes cure of said disease or condition, or any reduction or alleviation of disease, e.g. reduction in disease severity, or symptoms of disease. Exemplary parameters to assess might include reduction in mortality or intestinal lesion (necrotic lesion) score, or improved (or increased) final weights or reduced (or decreased or low) feed conversion ratio. Such increases or reductions might be in comparison to any appropriate control, examples of which are described herein, for example compared to untreated birds (e.g. birds untreated with the molecules of the invention).

The therapeutic methods and uses of the prevent invention are suitable for prevention of diseases as well as active treatment of diseases (for example treatment of pre-existing disease). Thus, prophylactic and metaphylactic (treating in the face of a disease outbreak, for example treating a group of subjects after the diagnosis of infection and/or clinical disease in part of the group, with the aim of preventing the spread of infectious disease to animals in close contact and/or at significant risk) treatment, in particular prophylactic treatment, is also encompassed by the invention. For this reason in the methods and uses of the present invention, treatment also includes prophylaxis, metaphylaxis or prevention where appropriate.

Such preventative (or protective) aspects can conveniently be carried out on healthy or normal or at risk subjects and can include both complete prevention and significant prevention. Similarly, significant prevention can include the scenario where severity of disease or symptoms of disease is reduced (e.g. measurably or significantly reduced) compared to the severity or symptoms which would be expected if no treatment is given.

Symptoms resulting from a clinical disease caused by or associated with NetB production, for example diseases such as NE which might be caused by infection with C. perfringens, include sudden increase in flock mortality, dehydration, apathy, diarrhea, wet litter, ruffled feathers and decreased feed consumption. Symptoms resulting from a sub-clinical disease include general poor performance and increased feed conversion ratio (feed conversion ratio is the amount of feed consumed/increase in body weight) as a consequence of chronic damage of the intestinal mucosa caused by the toxin. In addition, in subclinical forms, the damage to the intestine allows bacteria to reach the portal blood stream and colonize the liver causing diseased livers that will result in an increase of condemnations during processing at the slaughterhouse. Both clinical and sub-clinical forms of diseases, e.g. of NE, can be treated.

Suitable subjects for treatment in accordance with the present invention thus include any type of animal that is susceptible to infection with a pathogen that can produce NetB or a toxin similar to (or substantially homologous to) NetB. Suitable subjects thus typically include any type of animal that is susceptible to infection with C. perfringens, including human subjects. Preferred subjects include all types of avian or bird species, for example any domestic, farmed or wild avian or bird species, providing they are susceptible to or are capable of being infected with and suffering from diseases associated with NetB producing pathogens as defined herein, and in particular C. perfringens. Specific examples include subjects from the order Galloanserae. For example, preferred subjects are poultry animals or species such as chickens, turkeys, ducks, geese, pigeons, squab or quails. Chicken are especially preferred. Avian subjects can be suffering from clinical or subclinical forms of disease, e.g. NE. For the avoidance of doubt, the reference herein to a subject, e.g. the treatment of or administration to a subject, includes multiple subjects or a group of subjects.

In some embodiments, e.g. where prevention is concerned, the subject (or group of subjects) is a subject at risk of being affected by the disease or condition in question, for example at risk of being infected with a NetB producing pathogen (e.g. C. perfringens) as described above and developing disease. Such a subject may be a healthy subject or a subject not displaying any symptoms of disease or any other appropriate “at risk” subject. In another embodiment the subject is a subject having, or suspected of having (or developing), or potentially having (or developing) the disease or condition in question as described above.

Alternatively viewed, the present invention provides a method of treating or preventing a disease or condition associated with (or characterised by) NetB or where NetB has a role, for example a causative (e.g. a wholly or partially causative role) or an essential role, which method comprises administering to a subject (or group of subjects) in need thereof an effective amount of an anti-NetB antibody (or binding protein) of the invention, or a nucleic acid molecule, expression vector, host cell, or composition of the invention as defined herein, including combinations of agents. Appropriate diseases or conditions, and subjects, are described elsewhere herein.

The treatment or prevention of disease in avian species, in particular poultry, for example chickens, is preferred. Particularly preferred are methods for the treatment or prevention of C. perfringens infection. Particularly preferred are methods for the treatment or prevention of Necrotic enteritis (NE), for example NE caused by or associated with NetB production, in particular NetB production by C. perfringens.

Said anti-NetB antibodies (or binding proteins), etc., of the invention are generally administered in pharmaceutically or physiologically or therapeutically effective amounts, to a subject (or group of subjects) in need of same.

By “pharmaceutically or physiologically or therapeutically effective amount” is meant an amount sufficient to show benefit to the condition of the subject (or group of subjects). Whether an amount is sufficient to show benefit to the condition of the subject may readily be determined by a person skilled in the art. A pharmaceutically or physiologically or therapeutically effective amount can be determined based on clinical assessment and can be readily monitored

Embodiments of the therapeutic uses of the invention described herein apply, mutatis mutandis, to this aspect of the invention.

Further alternatively viewed, the present invention provides the use of an anti-NetB antibody (or binding protein) of the invention, or a nucleic acid molecule, expression vector, host cell, or composition of the invention as defined herein, including combinations of agents, in the manufacture of a medicament for use in therapy in a subject (or group of subjects), or for use in the treatment or prevention of a disease or condition in a subject (or group of subjects). Preferred therapeutic uses are described elsewhere herein, in particular for use in the treatment or prevention of any disease or condition associated with (or characterised by) NetB or where NetB has a role, for example a causative (e.g. a wholly or partially causative role) or an essential role. Appropriate diseases or conditions, and subjects, are described elsewhere herein.

Preferred embodiments provide the use of anti-NetB antibodies (or binding proteins), etc., of the invention in the manufacture of a medicament for use in the treatment or prevention of C. perfringens infection. The use of anti-NetB antibodies (or binding proteins), etc., of the invention in the manufacture of a medicament for use in the treatment or prevention of Necrotic enteritis (NE), for example NE caused by or associated with NetB production, in particular NetB production by C. perfringens are also preferred. The treatment or prevention of disease in avian species, in particular poultry, for example chickens, is preferred.

Embodiments of the therapeutic uses of the invention described herein apply, mutatis mutandis, to this aspect of the invention.

In some embodiments, the antibodies (or binding proteins) of the invention can be used in combination.

For example, the specific VHH antibodies, Net 83 and Net 14, as shown in Tables A and B, respectively, can be used in combination. Thus, two (or more) antibodies (or binding proteins) of the invention can be used in combination. One or more antibodies (or binding proteins) of the invention can also be used in combination with other active agents, including other active agents that target, inhibit or reduce the activity of NetB, for example other anti-NetB antibodies.

Alternatively in the above combinations, antibodies with the 3 CDRs as shown in Tables A and/or B, as appropriate, can also be used, or antibodies with substantially homologous CDR sequences as defined elsewhere herein.

Preferred combinations are those that result in improved or increased, preferably significantly improved or increased, therapeutic efficacy as compared to any of the antibodies (or binding proteins) of the invention (e.g. VHHs) administered as a sole active agent (monotherapy), or sole antibody, or sole anti-NetB agent. Other preferred combinations are those where the individual anti-NetB antibodies of the combination bind to different epitopes on the NetB molecule.

For such combination treatments using two or more anti-NetB antibodies (or binding proteins), the second (or subsequent) anti-NetB antibody may be administered to a subject substantially simultaneously with the first anti-NetB antibody, such as from a single pharmaceutical composition (and hence administered simultaneously) or from two pharmaceutical compositions administered closely together (at the same or a similar time). Alternatively, the second (or subsequent) anti-NetB antibody may be administered to a subject at a time prior to or sequential to the administration of the first anti-NetB antibody. “At a time prior to or sequential to”, as used herein, means “staggered”, such that the second antibody is administered to a subject at a time distinct to the administration of the first anti-NetB antibody component. Generally, the two (or more) components may be administered at times effectively spaced apart or together to allow the individual components to exert their respective therapeutic effects, i.e., they are administered in “biologically effective amounts” at “biologically effective time intervals” and are administered as part of the same therapeutic regimen.

Combinations of anti-NetB antibodies (or binding proteins), can, if appropriate, conveniently be administered as part of the same molecule or construct, e.g. can be conjugated or linked together, e.g. with an artificial linker. This mode of administration can be particularly appropriate for VHH antibodies (or other types of antibody molecule which are composed of a single polypeptide chain), individual antibodies of which can conveniently be connected by appropriate peptide (or other) linkers, e.g. non-native peptide or artificial linkers, in a single polypeptide chain containing multiple VHH (or other) antibodies, either of the invention or in combination with other VHHs or other antibodies, e.g. VHHs or other antibodies to other relevant entities, such as alpha-toxin (CPA). In such embodiments, agents are generally linked together using appropriate techniques, e.g. spacing, such that each component can exert their respective effects, for example binding to NetB. For example, in embodiments where anti-NetB antibodies bind to different epitopes on NetB, then combinations of such antibodies are preferred and the constructs are designed appropriately so that each individual antibody can bind to NetB, e.g. to its NetB epitope. In addition, multi-specific constructs, e.g. multi-specific VHH constructs, where specificities (preferably in the form of antibodies, e.g. VHH antibodies) other than (in addition to) NetB are present, e.g. in a single molecule, are also contemplated. Thus, combinations of anti-NetB antibodies (or binding proteins) of the invention can be combined with other antibodies that have specificities to entities other than NetB, e.g. antibodies to CPA.

In other embodiments of the invention, multiple copies of a single NetB antibody (or binding protein) of the invention can be provided on a single molecule, e.g. as a dimer (homodimer) or larger multimer (homomultimer), e.g. with three or four copies of the same antibody or binding protein. Thus, in preferred such embodiments a NetB antibody or binding protein of the invention, e.g. the Net 83 or Net 14 antibody as described herein, or an antibody based on the Net 83 or Net 14 antibody sequences set forth in Tables A or B as described herein, can be provided as a dimer or larger multimer. In such dimers or larger multimers the multiple copies of the antibodies or binding proteins can be linked in any appropriate way. Conveniently, especially for VHH antibodies (or other types of antibody molecule which are composed of a single polypeptide chain), individual antibodies can be connected in a single polypeptide chain by appropriate peptide (or other) linkers as described above. Such dimers or larger multimers form a yet further aspect of the invention.

Thus, in some embodiments the anti-NetB antibodies (or binding proteins) of the invention may be used as the sole active agent in a treatment regimen (monotherapy), or more than one of anti-NetB antibodies of the invention can be used in combination, for example as described above. In some embodiments the anti-NetB antibodies (or binding proteins) of the invention (or combinations as appropriate) may be used as the sole active anti-NetB agent(s) or the sole active anti-NetB antibodies in a treatment regimen, or they may be the sole active anti-C. perfringens or anti-NE agent(s) in a treatment regimen. However, in some embodiments, additional anti-NetB agents or anti-C. perfringens or anti-NE agents can be used.

Thus, the anti-NetB binding proteins or antibodies of the invention (or combination as appropriate) can be combined with one or more further (additional) NetB targeting or non-NetB targeting) active agents, e.g. with at least a second therapeutic or biological agent, where the anti-NetB binding protein or antibody of the invention (or combination of such binding proteins or antibodies), is the first.

The anti-NetB antibodies (or binding proteins) of the invention (or combination as appropriate) can for example be combined with any other therapeutic agent which is useful to treat the disease in question, for example the treatment of disease caused by or associated with C. perfringens infection or by NetB production, or the treatment of NE. Thus, the anti-NetB antibodies (or binding proteins) of the invention can be combined with other agents (e.g. other antibodies) that are used to treat C. perfringens infection, such as agents (e.g. antibodies) that inhibit other toxins that can be produced by C. perfringens, e.g. alpha toxin (CPA, C. perfringens alpha toxin). One or more alpha toxin antibodies, e.g. VHH antibodies or single chain antibodies, would be examples of an appropriate and preferred agent for use in such combination therapies, for example one or more anti-NetB VHH antibodies of the invention can be combined with one or more anti-alpha toxin (CPA) antibodies, e.g. VHH antibodies. Another example would be combinations with antibiotics, for example antibiotics that can be used for the treatment of disease caused by or associated with C. perfringens infection or by NetB production, or NE. Further examples would be combinations with probiotics or feed additives such as enzymes, vitamins and/or minerals. Further examples would be combinations with agents that can be used for the treatment of coccidiosis, such as coccidiostats or anti-coccidiosis antibodies. In all such combination treatments, e.g. a combination of an anti-NetB agent (such as an antibody or binding protein of the invention) and an anti-CPA agent, the therapeutic effects observed are preferably synergistic.

For such combination treatments, the second (non-anti-NetB antibody of the invention) agent may be administered to a subject together (simultaneously or substantially simultaneously, e.g. mixed together in a combined preparation, or as part of the same molecule, for example when two or more VHH antibodies or single chain antibodies are used) or separately as outlined above.

Thus, in some preferred embodiments, the anti-NetB antibodies (or binding proteins) of the invention are administered in combination with anti-CPA antibodies (or binding proteins). The different antibodies can for example be administered as a mixture, or as separate components, or can be provided as part of the same molecule, e.g. by linking the different antibodies together, e.g. an anti-NetB VHH of the invention can be linked to an anti-CPA VHH as described elsewhere herein.

Thus, a preferred composition of the invention comprises an anti-NetB antibody (or binding protein) of the invention in combination with an agent that inhibits CPA, preferably an anti-CPA antibody (or binding protein), e.g. a VHH antibody.

The invention further includes kits comprising one or more of the antibodies, or compositions of the invention, or one or more of the nucleic acid molecules encoding the antibodies of the invention, or one or more recombinant expression vectors comprising the nucleic acid sequences of the invention, or one or more host cells or viruses comprising the recombinant expression vectors or nucleic acid sequences of the invention. Preferably said kits are for use in the methods and uses as described herein, e.g. the therapeutic methods as described herein. Preferably said kits comprise instructions for use of the kit components. Preferably said kits are for treating diseases or conditions as described elsewhere herein, and optionally comprise instructions for use of the kit components to treat such diseases or conditions. Equivalent embodiments with binding proteins of the invention are also provided.

The antibodies (or binding proteins) of the invention as defined herein may also be used as molecular tools for in vitro or in vivo applications and assays, for example diagnostic assays. As the antibodies (and some binding proteins) have an antigen binding site, these can function as members of specific binding pairs and these molecules can be used in any assay where the particular binding pair member is required.

Thus, yet further aspects of the invention provide a reagent that comprises an antibody (or binding protein) of the invention as defined herein and the use of such antibodies (or binding proteins) as molecular tools, for example in in vitro or in vivo assays, for example for the detection of NetB, e.g. in a sample of interest.

In one embodiment, the invention provides a method of diagnosing NE or other diseases as described herein, in a subject, comprising the step of:
(a) contacting a test sample taken from said subject with one or more of the antibodies or binding proteins of the invention.

In a further embodiment, the invention provides a method of diagnosing NE or other diseases as described herein, in a subject, comprising the steps of:
(a) contacting a test sample taken from said subject with one or more of the antibodies or binding proteins of the invention;
(b) measuring or detecting the presence and/or amount and/or location of antibody-NetB or binding protein-NetB complexes in the test sample; and optionally
(c) comparing the presence and/or amount of antibody-NetB or binding protein-NetB complexes in the test sample to a control.

In the above methods, said contacting step is carried out under conditions that permit the formation (e.g. detectable formation) of antibody-NetB or binding protein-NetB complexes. Appropriate conditions can readily be determined by a person skilled in the art.

In the above methods any appropriate test sample or biological sample may be used, for example a blood or serum sample, material from tissues or organs suspected of being affected by NE or other diseases as described herein (e.g. small intestine or other appropriate GI samples), or histological sections. Samples are taken from appropriate subjects as defined elsewhere herein, e.g. avian subjects, e.g. poultry subjects such as chicken.

In certain of the above methods, the presence in the test sample of any amount of antibody-NetB or binding protein-NetB complexes would be indicative of the presence of NE or other diseases as described herein. Preferably, for a positive diagnosis to be made, the amount of antibody-NetB or binding protein-NetB complexes in the test sample is greater than, preferably measurably or significantly greater than, the amount found in an appropriate control sample (a control value or level). More preferably, the significantly greater levels are statistically significant, preferably with a probability value of <0.05. Appropriate methods of determining statistical significance are well known and documented in the art and any of these may be used.

Appropriate control samples could be readily chosen by a person skilled in the art. For example, in the case of diagnosis of NE, an appropriate control would be a sample from a subject that did not have NE, e.g. a healthy subject. Appropriate control “values” or “levels” could also be readily determined without running a control “sample” in every test, e.g. by reference to the range for normal or healthy subjects known in the art. The control value or level may thus correspond to the level in appropriate control subjects or samples, e.g. may correspond to a cut-off level or range found in a control or reference population.

In one embodiment the method of diagnosing NE or other diseases as described herein is an in vitro method.

In one embodiment the method of diagnosing NE or other diseases as described herein is an in vivo method.

Alternatively viewed, the present invention provides a method of screening for NE or other diseases as described herein, or a method of detecting NE or other diseases as described herein, in a subject.

The term “decrease” or “reduce” (or equivalent terms) as described herein includes any measurable decrease or reduction when compared with an appropriate control. Appropriate controls would readily be identified by a person skilled in the art and might include non-treated or placebo treated subjects or healthy subjects, or samples or assays where no antibody (or binding protein) of the invention is present. Preferably the decrease or reduction will be significant, for example clinically or statistically significant.

The term “increase” (or equivalent terms) as described herein includes any measurable increase or elevation when compared with an appropriate control. Appropriate controls would readily be identified by a person skilled in the art and might include non-treated or placebo treated subjects or healthy subjects, or samples or assays where no antibody (or binding protein) of the invention is present. Preferably the increase will be significant, for example clinically or statistically significant.

Preferably such increases (and indeed other increases, improvements or positive effects as mentioned elsewhere herein) or such decreases (and indeed other decreases, reductions or negative effects as mentioned elsewhere herein) are measurable increases, decreases, etc., (as appropriate), more preferably they are significant increases, decreases, etc., preferably clinically significant or statistically significant increases, for example with a probability value of 50.05 or <0.05, when compared to an appropriate control level or value (e.g. compared to an untreated or placebo treated subject or compared to a healthy or normal subject, or the same subject before treatment).

Methods of determining the statistical significance of differences between test groups of subjects or differences in levels of a particular parameter are well known and documented in the art. For example herein a decrease or increase in level of a particular parameter or a difference between test groups of subjects is generally regarded as statistically significant if a statistical comparison using a significance test such as a Student t-test, Mann-Whitney U Rank-Sum test, chi-square test or Fisher's exact test, one-way ANOVA or two-way ANOVA tests as appropriate, shows a probability value of:50.05 or <0.05.

Tables of Amino Acid Sequences Disclosed Herein and their Sequence Identifiers (SEQ ID NOs)

All amino acid sequences are recited herein from the N-terminus to the C-terminus in line with convention in this technical field.

TABLE A

| | | |
|---|---|---|
| SEQ ID | | |
| NO: | Description | Sequence |

Net 83

| | | |
|---|---|---|
| 1 | VHH (aa) | QVQLVESGGGLVQAGESL |
| | | RLSCAASGSIRSISFMNW |
| | | YRQQPGKQRELVAGINQI |
| | | GNTNYAGSVKGRFTISRD |
| | | NAKNTVYLQMNSLKPEDT |
| | | AVYYCNVIVRGTVSPFQF |
| | | WGQGTQVTVSS |
| 2 | Heavy CDR1 | GSIRSISFMN |
| 3 | Heavy CDR2 | INQIGNT |
| 4 | Heavy CDR3 | IVRGTVSPFQF |
| 5 | Heavy FR1 | QVQLVESGGGLVQAGESL |
| | | RLSCAAS |
| 6 | Heavy FR2 | WYRQQPGKQRELVAG |
| 7 | Heavy FR3 | NYAGSVKGRFTISRDNAK |
| | | NTVYLQMNSLKPEDTAVY |
| | | YCNV |
| 8 | Heavy FR4 | WGQGTQVTVSS |

TABLE B

| | | |
|---|---|---|
| SEQ ID | | |
| NO: | Description | Sequence |

Net 14

| | | |
|---|---|---|
| 9 | VHH (aa) | QVQLVESGGGLVQAGGS |
| | | VRLSCVASGLTFSGYSL |
| | | GWFRQAPGKEREGVSYI |
| | | SSSGMITDYADSVKGRV |
| | | TISRDDAKSTMYLQMNS |
| | | LKPEDTAVYYCAARFPR |
| | | PRSWLSTDSYNYWGQGA |
| | QVTVSS | |
| 10 | Heavy CDR1 | GLTFSGYSLG |
| 11 | Heavy CDR2 | ISSSGMIT |
| 12 | Heavy CDR3 | RFPRPRSWLSTDSYNY |
| 13 | Heavy FR1 | QVQLVESGGGLVQAGGS |
| | | VRLSCVAS |
| 14 | Heavy FR2 | WFRQAPGKEREGVSY |
| 15 | Heavy FR3 | DYADSVKGRVTISRDDA |
| | | KSTMYLQMNSLKPEDTA |
| | | VYYCAA |
| 16 | Heavy FR4 | WGQGAQVTVSS |

NetB toxin
(SEQ ID NO: 17)
SELNDINKIELKNLSGEIIKENGKEAIKYTSSDTASHKGWKATLS
GTFIEDPHSDKKTALLNLEGFIPSDKQIFGSKYYGKMKWPETYRI
NVKSADVNNNIKIANSIPKNTIDKKDVSNSIGYSIGGNISVEGKT
AGAGINASYNVQNTISYEQPDFRTIQRKDDANLASWDIKFVETKD
GYNIDSYHAIYGNQLFMKSRLYNNGDKNFTDDRDLSTLISGGFSP
NMALALTAPKNAKESVIIVEYQRFDNDYILNWETTQWRGTNKLSS
TSEYNEFMFKINWQDHKIEYYL
NetB toxoid
(SEQ ID NO: 18)
SELNDINKIELKNLSGEIIKENGKEAIKYTSSDTASHKGWKATLS
GTFIEDPHSDKKTALLNLEGFIPSDKQIFGSKYYGKMKWPETYRI
NVKSADVNNNIKIANSIPKNTIDKKDVSNSIGYSIGGNISVEGKT
AGAGINASYNVQNTISYEQPDFRTIQRKDDANLASWDIKFVETKD
GYNIDSYHAIYGNQLFMKSRLYNNGDKNFTDDRDLSTLISGGFSP
NMALALTAPKNAKESVIIVEYQRFDNDYILNWETTQARGTNKLSS
TSEYNEFMFKINWQDHKIEYYL

The invention will now be further described in the following non-limiting Examples with reference to the following drawings:

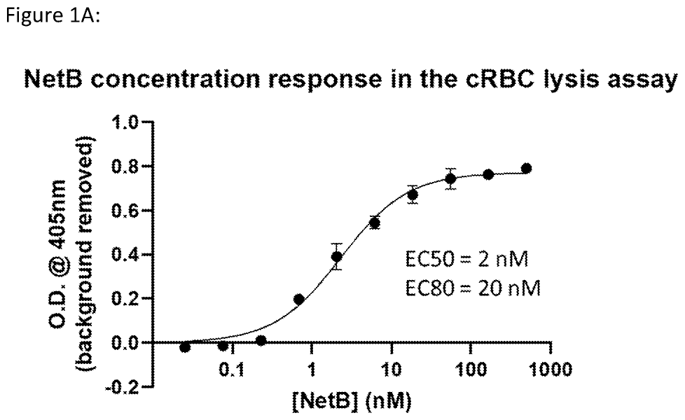

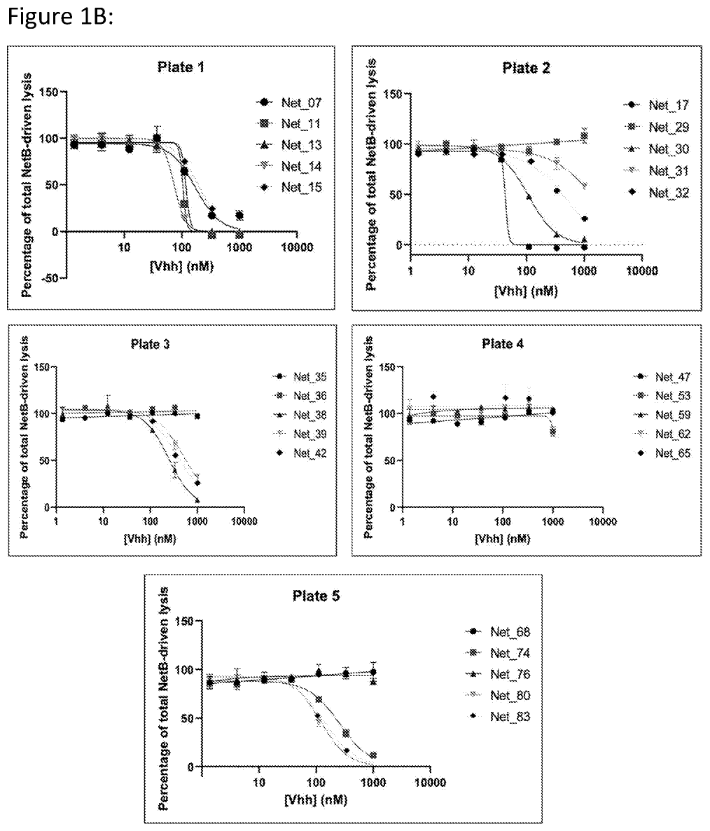

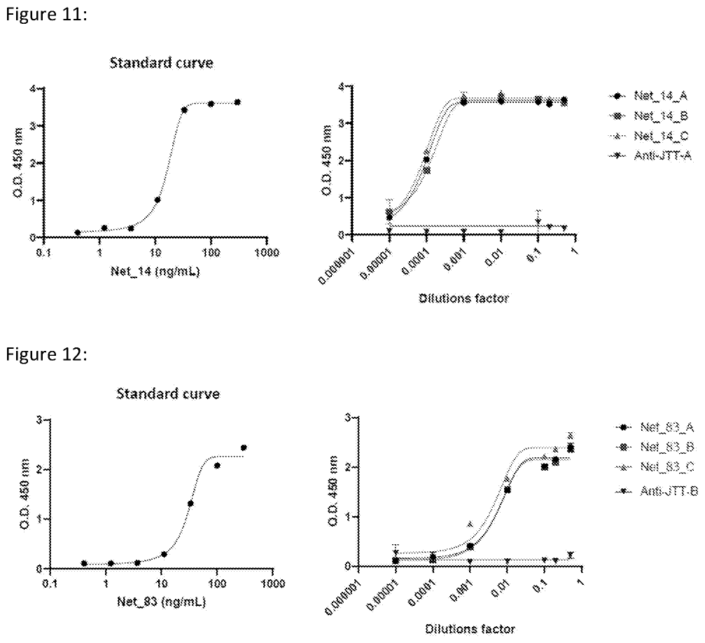

FIG. 1A shows a standard curve of NetB-induced red-blood cell lysis. FIG. 11B shows Inhibition of NetB induced chRBC lysis assay (pH7.4).

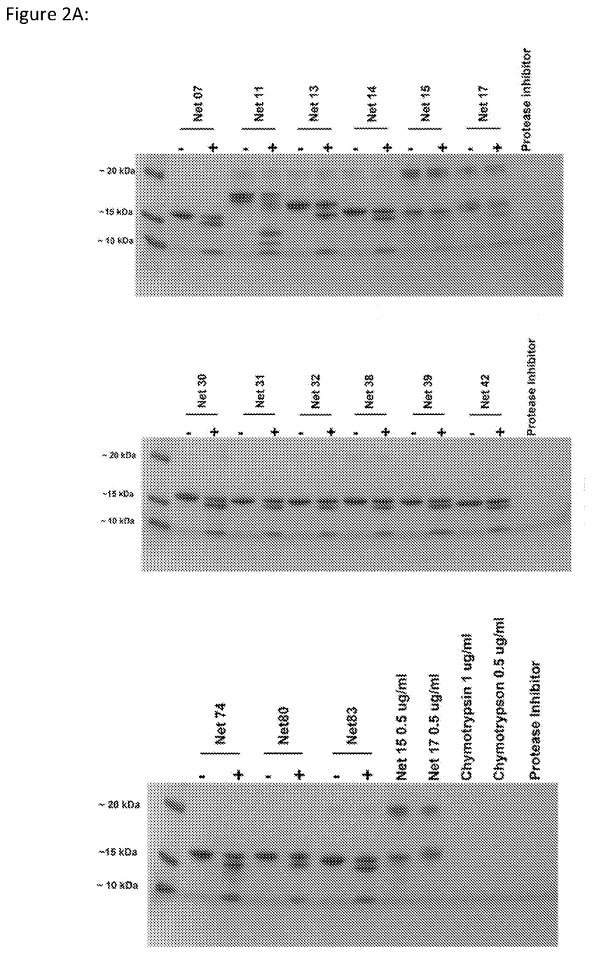

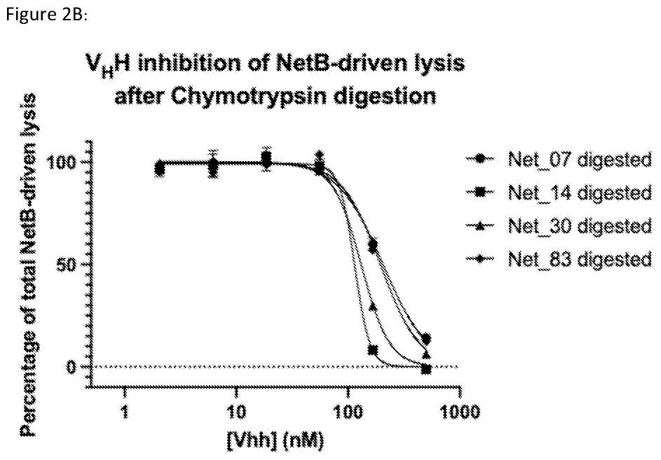

FIG. 2A shows stability after incubation with Chymotrypsin. SDS-PAGE gel of VHH after incubation with Chymotrypsin. FIG. 2B shows activity of selected clones after incubation with Chymotrypsin.

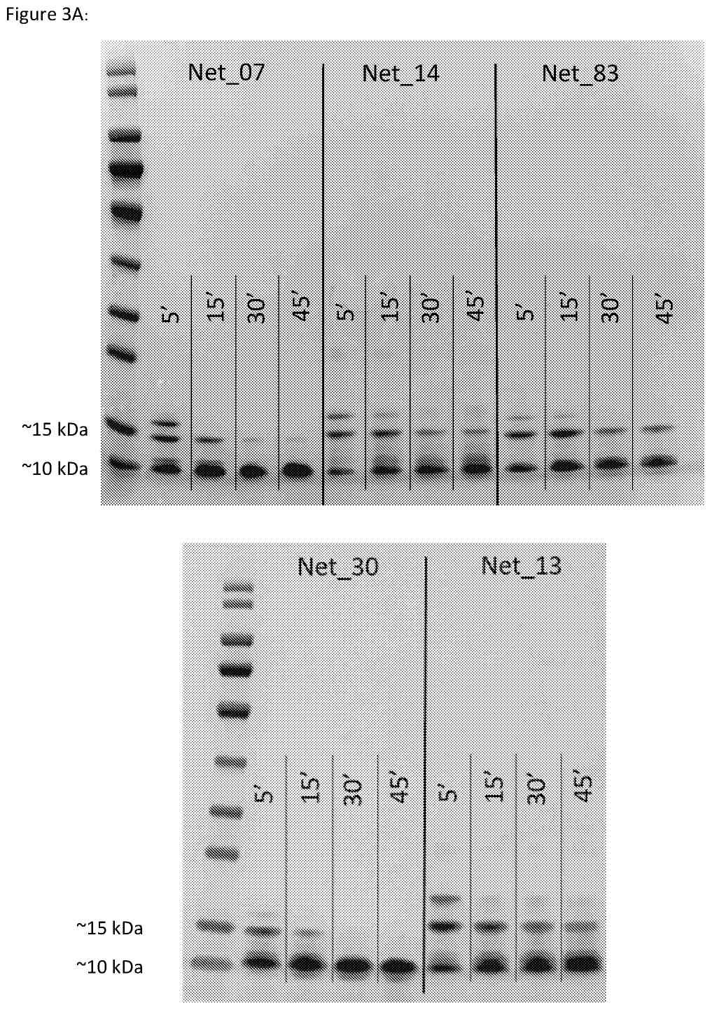

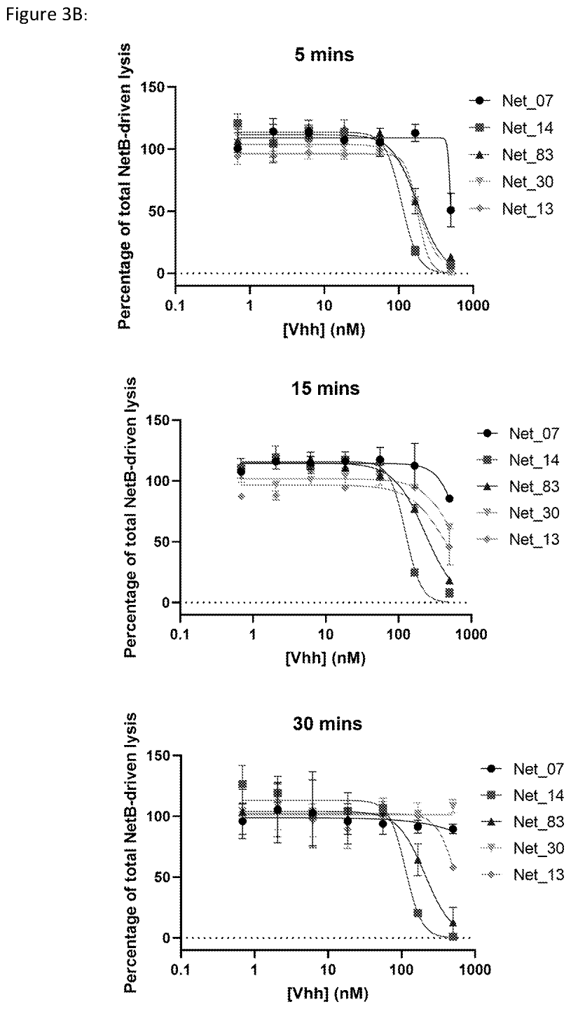

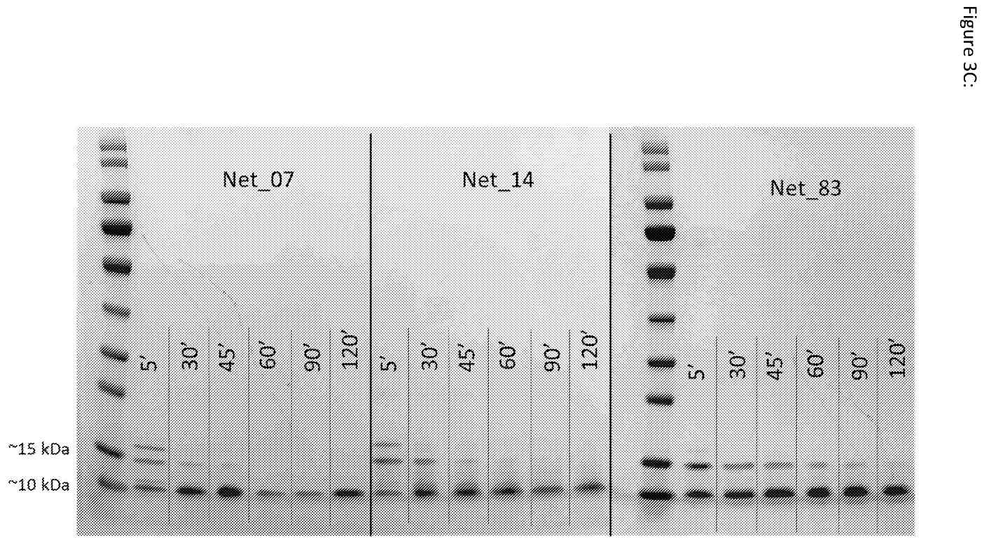

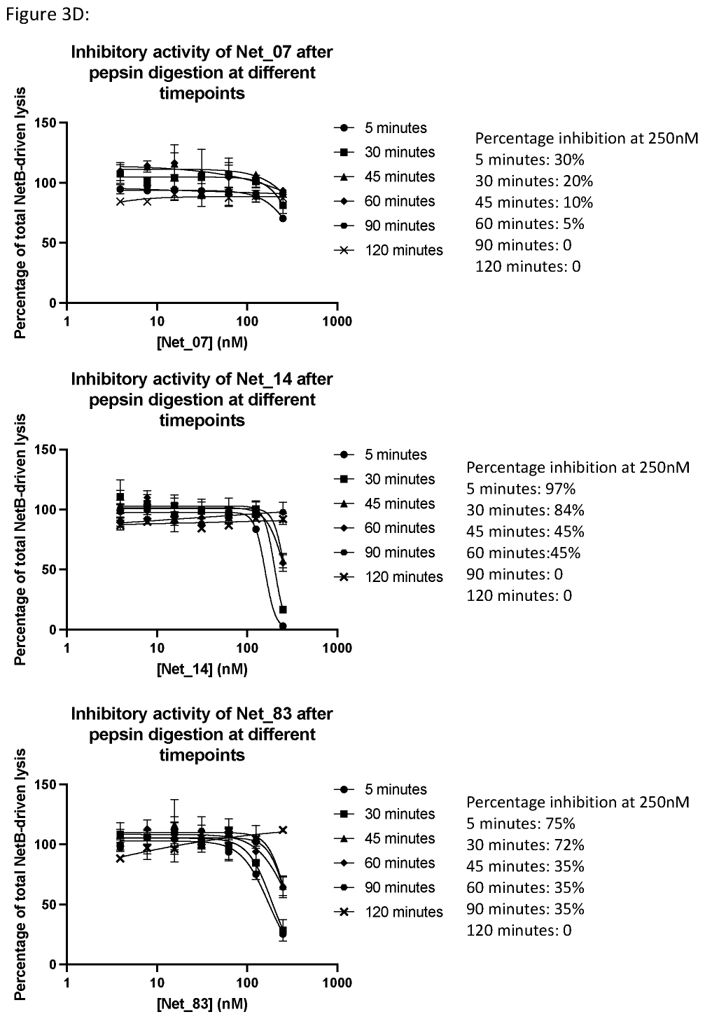

FIG. 3A shows stability and activity after incubation with Pepsin. FIG. 3B shows activity after incubation with Pepsin. Inhibition of NetB-induced RBC-lysis by VHH after incubation with Pepsin. FIG. 3C shows stability of select clones after incubation with Pepsin. SDS-PAGE gel of select VHH at various timepoints during an extended incubation with Pepsin. FIG. 3D shows activity of select clones after incubation with Pepsin.

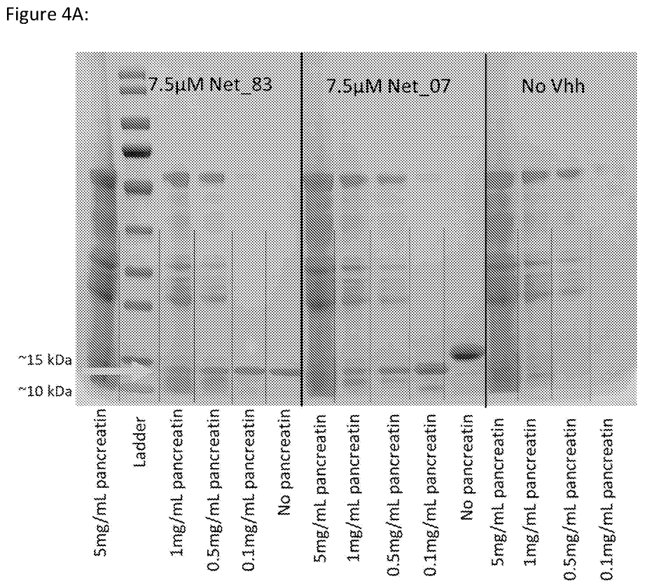

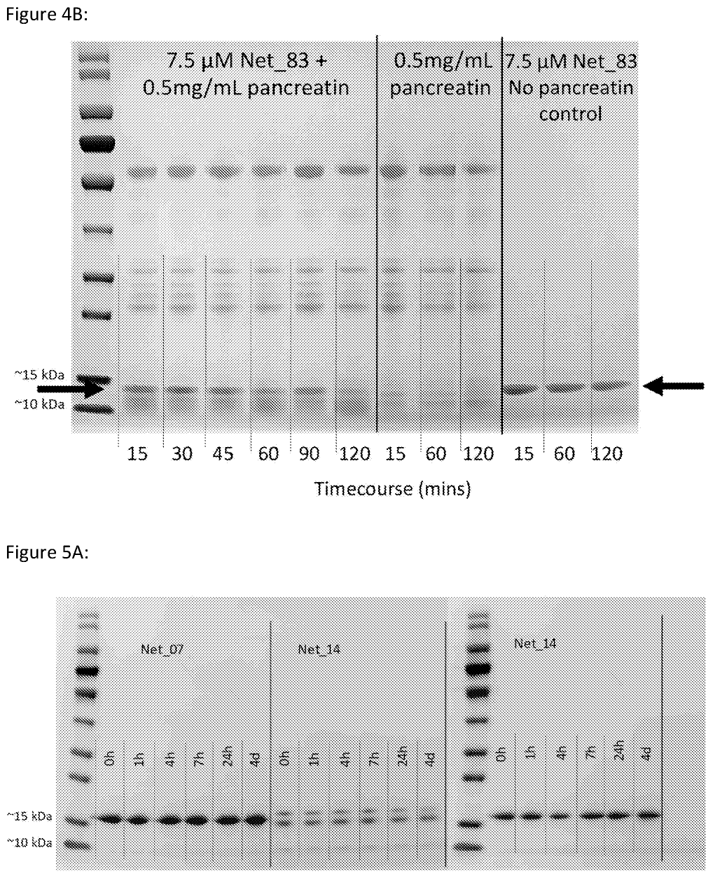

FIG. 4A shows stability after incubation with Pancreatin. SDS-PAGE gel of VHH incubated with increasing amounts of Pancreatin. FIG. 4B shows stability of selected VHH after incubation with Pancreatin. SDS-PAGE gel of VHH at various timepoints during incubation with Pancreatin.

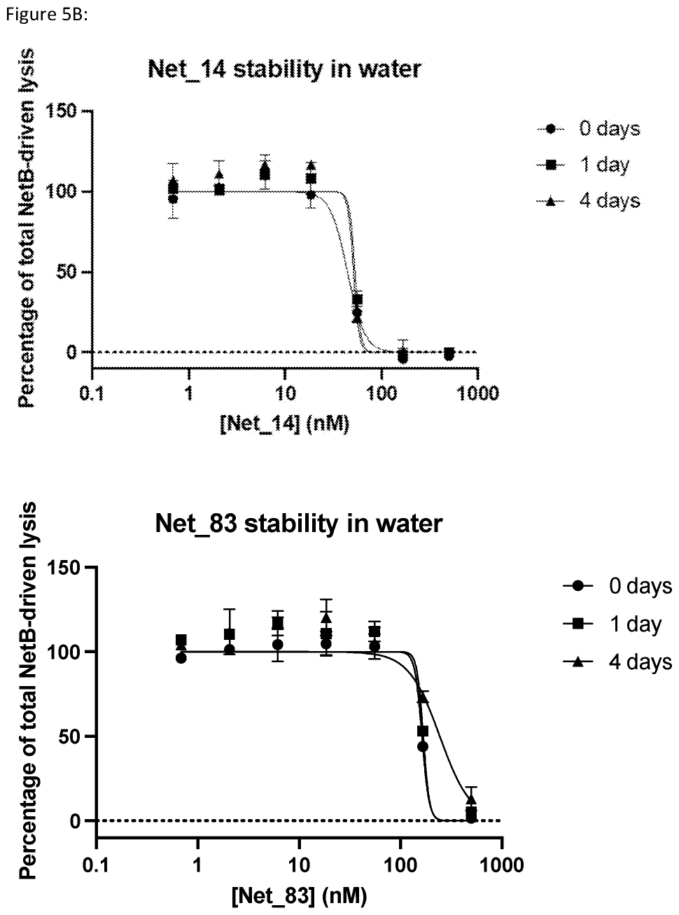

FIG. 5A shows a gel evidencing VHH stability in water. FIG. 5B shows VHH stability in water and effect on potency

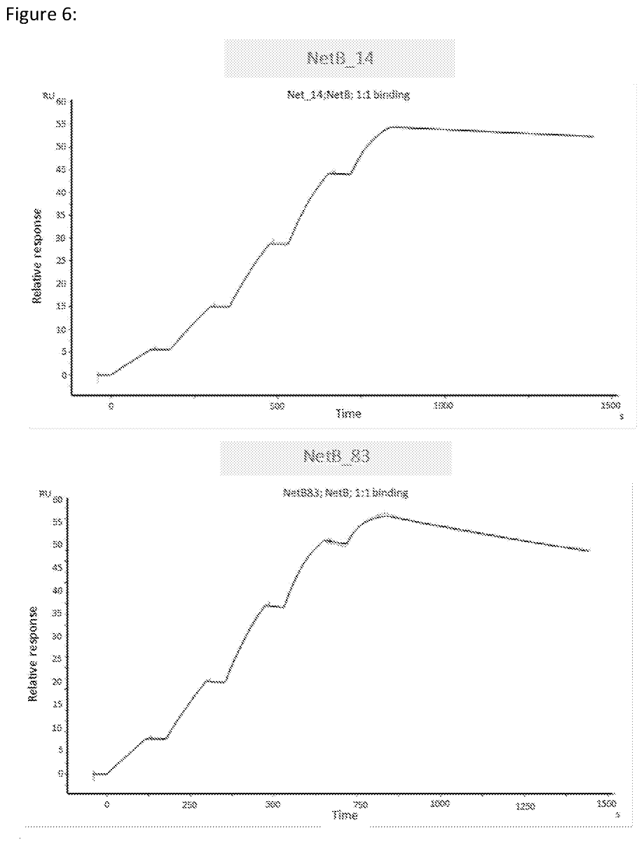

FIG. 6 shows Biacore titration of selected VHH.

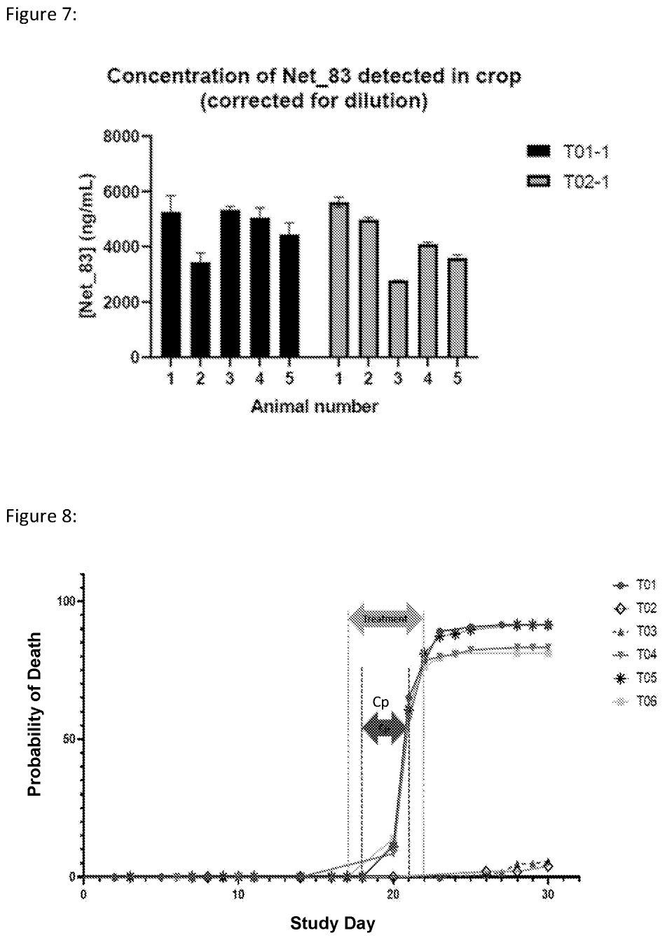

FIG. 7 shows the concentration of Net_83 in digesta samples from the crop of birds receiving water supplemented with the antibody ad libitum for 24 hours T01−1=Net_83, T02−1=Net_83 in water with NaHCO3

FIG. 8 shows the probability of death due to necrotic enteritis across causes from Clostridium perfringens (Cp) challenge to study end (day 17-day30). Treatment with antibodies was for 6 days. T01=infected untreated control (IUC), T02=unchallenged untreated (UUC), T03=Amoxicillin in drinking water for 5 days, T04=CPA+Net_83 antibodies, T05=NaHCO3, T06=CPA+Net_83 antibodies in water with NaHCO3

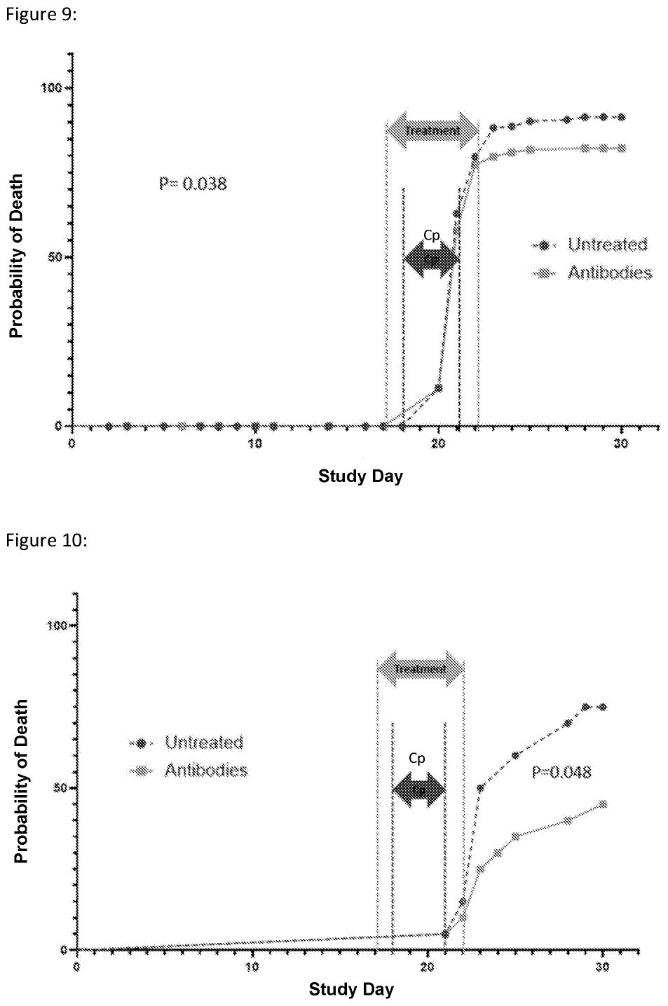

FIG. 9 shows the probability of bird death of antibody groups (T04 & T06) combined (n=260) vs. T01 (IUC) and T05 (NaHCO3) combined (Untreated group; n=260). Log-Rank P-value for time to event analysis in each group is shown. A bird was censored when it survived until day 30.

FIG. 10 shows the probability of having a death pen for antibody groups (T04 & T06) combined (n=20) vs. TO1 (IUC) and T05 (NaHCO3) combined (untreated group, n=20). A pen was considered to survive if it still was housing at least one bird on day 30. A pen was classed as dead when it lost all birds due to NE. Log-Rank P-value for time to event analyses is provided.

FIG. 11 shows the ELISA binding to NetB-His of the supernatant of three Bacillus subtilis clones expressing Net_14, and one expressing an irrelevant VHH clone (Anti-JTT-A)

FIG. 12 shows the ELISA binding to NetB-His of the supernatant of three Bacillus subtilis clones expressing Net_83, and one expressing an irrelevant VHH clone (Anti-JTT-B)

### EXAMPLES

### Example 1: Immunisation, Library Generation, Screening and Clone Selection

### Materials and Methods

### Immunisations

Single domain antibodies were obtained from llamas immunized with recombinant protein. Llamas were injected with inactive Clostridium perfringens NetB toxoid antigen formulated in Incomplete Freund's Adjuvant. Animals were immunized with six subcutaneous injections (two injections with 100 μg/dose followed by four injections with 50 μg/dose) at weekly intervals. One week after the last boost, sera were collected to define antibody titers against NetB toxoid.

In this ELISA, 96-well plates (Maxisorp; Nunc) were coated with the recombinant protein. After blocking and adding diluted sera samples, the presence of anti NetB toxoid antibodies was demonstrated by using mouse anti-llama IgG (FJB, Cat. nr. FJ1203MAB01B09) followed by an anti-mouse immunoglobulin peroxidase conjugate (JIR, Cat. nr. 715-035-150).

### Library Build & Selections

RNA was extracted from PBMC of 3 immunized llamas (400 ml each). 40 μg of RNA was used for cDNA synthesis using random primers. The cDNA was used in a primary PCR amplification using non-tagged primers annealing at the Leader sequence and Hinge CH1 regions, followed by a secondary PCR amplification introducing restriction endonuclease sites for cloning of VHH genes in pDCL1 phagemid vector. The libraries were electroporated into TG1 E. coli cells and bacterial glycerol stock of the immune libraries were stored at −80° C. (FL2471 and FL2472)

### Selections

Phage production from the llama VHH library pool were used in three consecutive rounds of phage display selection using NetB toxoid recombinant protein. Selection rounds on recombinant proteins were performed using 10 μg/ml of NetB toxoid (PBS buffer) with washing of non-specific phage, followed by specific phage elution with trypsin (total elution).

Serial dilutions of the eluted phages were performed and used to infect exponentially growing TG1. Infected TG1 was plated on LBCarb100Glu2% plates and enrichment values calculated over the background (without antigen for selection).

### Primary Screening

### Screening for Binding by ELISA

Individual clones from the first and second rounds of selection condition outputs were picked into 96-well Master Plates and tested as Periplasmic Extract (P.E.) for binding to NetB toxoid at pH 7.4 via binding ELISA. For P.E. binding ELISA, MaxiSorp™ high protein-binding capacity 96 well ELISA plates, were coated with 1 μg/ml of NetB toxoid, diluted in PBS, overnight at 4° C. The next day, plates were washed 3× with PBS Tween 0.05% (pH 7.4) and blocked for 1 hour at room temperature with 250 μl/well of 4% Marvel/CPA or 4% Marvel/PBS. After blocking, plates were washed 3× with PBS Tween 0.05% (pH7.4) and incubated per well with 20 μl of P.E+80 μl in 1% Marvel/PBS (pH7.4), for 1 hour at RT with shaking. Plates were washed 3× with PBS Tween 0.05% (pH7.4) and incubated with 100 μl of anti-c-Myc antibody (Roche; Cat. nr. 11667203001) followed by secondary antibody DAM-HRP (JIR; Cat. nr. 715-035-150) in 1% Marvel/PBS (pH7.4), for 1 hour at RT with shaking. Plates were washed 3× with PBS Tween 0.05% (pH7.4) and the substrate solution (TMB solution) was added to the plates. Reaction was stopped with H₂SO₄ and plates read in the plate reader at 450 nm.

### Sequencing

The positive binders were sent to be sequenced. Clones were classified by families according to the different HCDR3 sequence.

### Results & Discussion

Following immunisation of llama with recombinant NetB toxoid, phage clones were selected by 2 rounds of phage panning. Phage candidates were confirmed by periplasmic extract binding recombinant NetB toxoid by ELISA. Binding was demonstrated by several candidates to NetB toxoid, as demonstrated in Table 1, which shows data from a subset of clones shown to bind to the NetB toxoid.

TABLE 1
Periplasmic Extract ELISA Binding Data Summary

| | PE ELISA |
|---|---|
| | pH 7.4 |
| Clone ID | OD450 |
| Net_07 | 4.54 |
| Net_11 | 4.53 |
| Net_13 | 4.60 |
| Net_14 | 4.67 |
| Net_15 | 4.60 |
| Net_17 | 4.22 |
| Net_30 | 4.57 |
| Net_31 | 4.35 |
| Net_32 | 4.36 |
| Net_38 | 4.58 |
| Net_42 | 4.27 |
| Net_74 | 4.42 |
| Net_80 | 4.51 |
| Net_83 | 4.49 |

Clones were further screened and selected for their off-rate when binding to recombinant NetB toxoid by Biacore. NetB toxoid protein immobilized on CM5 sensor chip in 10 mM of Sodium Acetate pH 5.0 at approximately 1100 RU by amine coupling. P.E. of selected clones were diluted 1:5 in HBS-EP pH 7.4 buffer and injected over the immobilised NetB toxoid at 30 μl/min for 2 min. After this binding injection, samples were left to dissociate for 5 mins. The chip was regenerated between runs with one injection of 1 M NaCl/1 mM glycine pH 1.5. Controls included an Anti-histidine antibody (BioLegend, Cat. nr. 652502) at 5 μg/ml in HBS-EP pH 7.4 buffer, which showed no significant decrease in binding at the beginning and ends of the runs. An irrelevant VHH P.E. (FJ1409MPO3C04) run in the same way showed no binding to NetB toxoid. Binding with good off-rates was demonstrated by several candidates to NetB toxoid, as demonstrated in Table 2.

Hence, the immunisation of llama with recombinant NetB toxoid protein resulted in the successful isolation of phage antibody candidates able to bind NetB with good off-rates.

TABLE 2
Periplasmic extract off-rate binding data

| | Antigen | Antigen |
|---|---|---|
| Clone | NetB-His | NetB-His |
| ID | RUmax (RU) | kd (1/s) |
| Net_07 | 222.6 | 6.77E−04 |
| Net_11 | 89.8 | 3.53E−04 |
| Net_13 | 181.0 | 8.93E−04 |
| Net_14 | 335.4 | 1.43E−04 |
| Net_15 | 343.8 | 1.03E−03 |
| Net_17 | 192.4 | 6.67E−05 |
| Net_30 | 326.8 | 6.45E−04 |
| Net_31 | 226.0 | 1.40E−02 |
| Net_32 | 290.8 | 2.00E−03 |
| Net_38 | 328.5 | 1.28E−03 |
| Net_42 | 194.3 | 2.67E−03 |
| Net_74 | 312.9 | 1.17E−03 |
| Net_80 | 260.8 | 8.58E−04 |
| Net_83 | 328.6 | 4.27E−04 |

### Example 2: Confirmation of Binding of Purified VHH Against NetB Toxin at pH 7.4

### Assessing Binding at pH 6.0

### Material and Methods

### Expression and Purification of VHH Candidate Antibodies

The synthetic genes codifying to the VHH variable domains with Avi-tag and His tags were purchased cloned into pET15b bacterial expression vector with a periplasmic secretion leader sequence. E. coli strain BL21 DE3 were transformed and grown in ZYP-5052 autoindution media for 68 hours at 18° C. Produced VHH antibodies were captured from clarified supernatants using Ni-NTA beads (Qiagen) on gravity fed columns. Eluted antibodies were buffer exchanged to 1×PBS pH 7.4 and concentrated using 10k cutoff spin concentrators (Amicon, Cat. nr. UFC801096D). Purified VHH protein was analysed by SDS-PAGE for the presence of correct chains.

### Screening for Binding by ELISA

Individual clones were tested as purified VHH for binding to NetB toxin at pH 6.0 and pH 7.4 via binding ELISA. For VHH binding ELISA, MaxiSorp™ high protein-binding capacity 96 well ELISA plates, were coated with 1 μg/ml of NetB toxin, diluted in phosphate buffered saline (PBS) at pH7.4, or citrate-phopshate buffer (CPB) at pH6.0 overnight at 4° C. The next day, plates were washed 3× with PBS Tween 0.05% (pH 7.4) and blocked for 1 hour at room temperature with 100 μl/well of 4% Marvel/CPB or 4% Marvel/PBS. After blocking, plates were washed 3× with PBS Tween 0.05% (pH7.4) or CPB Tween 0.05% (pH6.0) and incubated per well with 100 μl of VHH at 180 nM in PBS (pH7.4) or CPB (pH6.0), for 1 hour at RT. Plates were washed 3× with PBS Tween 0.05% (pH7.4) or CPB Tween 0.05% (pH6.0) and incubated with 50 μl of anti-Strep II antibody (Abcam; Cat. nr. Ab252885) followed by secondary antibody Goat anti-rat IgG−HRP (Alpha diagnostic International; cat. Nr 50320-200) in PBS (pH7.4) or CPB (pH6.0), for 1 hour at RT. Plates were washed 3× with PBS Tween 0.05% (pH7.4) or CPB Tween 0.05% (pH6.0) and the substrate solution (TMB solution) was added to the plates. Reaction was stopped with H₂SO₄ and plates read in the plate reader at 450 nm.

### Results & Discussion

Binding of the purified VHH to NetB toxin was confirmed for many clones at both pH 7.4 and the more physiologically relevant pH 6.0, as demonstrated in Table 3.

TABLE 3
Purified VHH ELISA Binding Data Summary

| | VHH | VHH |
|---|---|---|
| | ELISA pH | ELISA pH |
| Clone | 6.0 | 7.4 |
| ID | OD450 | OD450 |
| Net_07 | 1.468 | 0.644 |
| Net_11 | 1.995 | 0.747 |
| Net_13 | 0.912 | 0.283 |
| Net_14 | 1.531 | 0.412 |
| Net_15 | 1.586 | 0.737 |
| Net_17 | 1.344 | 0.186 |
| Net_30 | 1.524 | 0.672 |
| Net_31 | 0.453 | 0.314 |
| Net_32 | 1.162 | 0.609 |
| Net_38 | 1.068 | 0.417 |
| Net_42 | 1.033 | 0.511 |
| Net_74 | 0.993 | 0.658 |
| Net_80 | 1.113 | 0.787 |
| Net_83 | 1.102 | 0.756 |

### Example 3: Inhibition of NetB Induced chRBC Lysis Assay

### Material and Methods

### Expression and Purification of VHH Candidate Antibodies

Carried out as described above in Example 2.

### Standard Curve of NetB-Induced Red-Blood Cell Lysis

### Preparing Red Blood Cells (RBC)

3 mL of well-resuspended fresh chicken blood (TCS Biosciences; Cat #FB011AP) is washed by adding to 25 ml of PBS, mixed gently, and centrifuged at 500×g at RT. Supernatant is removed and the pellet resuspended in 25 mL PBS before another wash step. After the second wash step, the RBC pellet is resuspended once more in 20 ml PBS. The OD595 is measured and the volume adjusted to have the OD595=0.5.

### NetB Induced Red Blood Cell Lysis Assay

A serial dilution of active NetB toxin is made in PBS and added 1:1 to RBCs (as prepared above at OD595=0.5). The NetB toxin and RBCs are incubated at 37° C. for 30 mins, shaking. After incubation, the plate is centrifuged at 1100×g for 3 mins. 50 ul of each of the samples is removed to a suitable flat-bottom plate, being careful not to disturb the RBC pellet. The OD405 is recorded for the plate by a spectrophotometer.

### Characterisation of NetB Toxin and Determination of Sub-Maximal Dose (EC80)

Purified NetB toxin is characterised with each fresh lot of chicken blood and prior to running the inhibition of lysis assays. The concentration response is measured for NetB's ability to lyse chicken red blood cells (data not shown). The calculated EC80 (20 nM) is used for the assays examining inhibition of NetB induced RBC lysis assay by the VHH.

### Preparation of VHH for the Inhibition of NetB-Induced Red Blood Cell Lysis

VHH are diluted from 4 uM down to 5.49 nM in 3-fold serial dilutions and 17.5 uL transferred into a v-bottom 96 well assay plate. Then 17.5 uL NetB (diluted in PBS to 4× the EC80) is added to the respective wells. For the positive control, NetB only was used. The negative control contained no NetB. The NetB and VHH clones (35 uL total volume) are incubated in the assay plate for 15 minutes at room temperature prior the addition of 35 μL of washed and prepared RBCs. The assay plate is sealed with an adhesive plate seal, and then incubated at 37° C. for 30 mins, shaking (450 rpm). After incubation, the plate is centrifuged at 1100×g for 3 mins and 50 ul of each of the samples is removed to a suitable flat-bottom plate, being careful not to disturb the RBC pellet. The OD405 is recorded for the plate by a spectrophotometer.

### Results & Discussion

FIG. 1A shows the standard curve of NetB-induced red-blood cell lysis. The EC50 of the NetB toxin in these assays is 2 nM. The sub-maximal concentration of NetB used in these assays is equivalent to the EC80=20 nM.

The final range of VHH concentrations used in the assay is from 1 uM down to 1.37 nM This equates to range of 50:1 down to 0.0685:1 molar ratio (VHH:NetB).

A number of VHH exhibited the potential to inhibit NetB lysis activity as shown in Table 4.

FIG. 1B shows the titration inhibition curves for various VHH screened at pH 7.4. Several showed potent inhibition of NetB-induced lysis. IC50 and molar ratios are reported in Table 4. The VHH were also screened at pH 6.0. IC50 and molar ratios are reported in Table 4.

It can be seen that some of the VHH clones advantageously show improved bioactivity at pH 6.0 as compared to pH 7.4.

TABLE 4
Inhibition of NetB-induced red blood
cell (RBC) lysis (pH 7.4 and pH 6.0)

| | | | | Bioactivity |
|---|---|---|---|---|
| | Bioactivity | Bioactivity | Bioactivity | pH 6.0 |
| | pH 7.4 | (Molar ratio to | pH 6.0 | (Molar ratio to |
| Clone ID | (IC50, nM) | NetB @ 20 nM) | (IC50, nM) | NetB @ 20 nM) |
| Net_07 | 172.30 | 8.62 | 31.14 | 1.56 |
| Net_11 | 104.90 | 5.25 | 38.26 | 1.91 |
| Net_13 | 118.50 | 5.93 | 264.30 | 13.22 |
| Net_14 | 73.96 | 3.70 | 32.25 | 1.61 |
| Net_15 | 197.90 | 9.90 | 418.60 | 20.93 |
| Net_17 | 43.38 | 2.17 | 55.03 | 2.75 |
| Net_30 | 107.40 | 5.37 | 121.60 | 6.08 |
| Net_31 | 1454.00 | 72.70 | 1105.00 | 55.25 |
| Net_32 | 457.40 | 22.87 | 774.00 | 38.70 |
| Net_38 | 246.60 | 12.33 | 708.90 | 35.45 |
| Net_42 | 400.60 | 20.03 | 835.10 | 41.76 |
| Net_74 | 259.70 | 12.99 | 412.50 | 20.63 |
| Net_80 | 121.20 | 6.06 | 351.20 | 17.56 |
| Net_83 | 137.60 | 6.88 | 102.60 | 5.13 |

In subsequent experiments, the above chRBC lysis assay was run with Net_14 and Net_83 at a temperature of 42° C. instead of 37° C. to more closely mimic the body temperature of a chicken. The results showed that NetB shows similar potency at 42° C. and 37° C. and both VHH antibodies retain the ability to inhibit NetB activity at this elevated temperature.

### Example 4: Stability and Activity after Incubation with Chymotrypsin (Resistance to Chymotrypsin)

Various purified VHH candidates that inhibited NetB-induced red blood cell lysis were screened for their ability to resist the proteases found in the gastrointestinal (GI) tract.

Candidate VHH were incubated with Chymotrypsin (1 ug/ml) for 1 h prior to stopping the reaction and the VHH run on an SDS-PAGE gel and assessed for their continued ability to inhibit NetB induced red blood cell lysis.

### Material and Methods

Incubation of VHH with Chymotrypsin

VHH were prepared at 15 μM in PBS (except where VHH were at starting concentrations <15 uM, in which case they were used at as high a concentration as available). A stock concentration of chymotrypsin (Sigma Aldrich; cat no. 11418467001) was made at 100 μg/mL in 1 mM HCl by adding 250 μL 1 mM HCl to a vial containing 25 pg chymotrypsin.

Chymotrypsin was then further diluted in PBS to a sub-stock concentration of 10 μg/mL. 18 μL of the prepared VHH was added to low-protein binding PCR tubes. 2 μL of 10 μg/mL chymotrypsin (or 2 μL PBS for controls) was added to each tube. The tubes are then spun down for 15 seconds to ensure chymotrypsin is added at the same time for all tubes.

PCR tubes are then transferred to a heat block for 1 h at 37° C. to carry out the digestion before being stopped by the addition of 3.3 μL (7×) protease inhibitor and transferring to ice.

SDS-PAGE Gel of VHH after Incubation with Chymotrypsin

5 μL of each stopped VHH sample is taken and added to 2 μL of 5×SDS loading buffer added to each tube, and the tubes sealed with a foil adhesive plate seal. The tubes are transferred to a heat block and incubated at 80° C. for 10 minutes before being spun down to collect any evaporate. The denatured samples are run out on a 4-20% graduated SDS-PAGE gel. After the gel has run, it is stained with Coomassie staining reagent for 24 h, then destained with water before pictures are taken to assess degradation after digestion with chymotrypsin.

NetB-Induced Red Blood Cell Assay with VHH Incubated with Chymotrypsin

10 μL of each stopped VHH sample is taken and added to 65 μL PBS, before being diluted 1 in 3 from 2 uM down to 5.49 nM. These dilutions are at 4× final concentration to allow for dilution upon addition of chRBCs and NetB toxin. NetB will be used at the sub-maximal concentration (EC80) of 20 nM. The maximum final concentration of VHH will be 500 nM=25:1 (VHH:NetB) molar ratio, and the lowest concentration will be 1.37 nM=0.0685:1 molar ratio (VHH:NetB)

VHH are diluted from 2 uM down to 5.49 nM in 3-fold serial dilutions and 17.5 uL transferred into a v-bottom 96 well assay plate. Then 17.5 uL NetB (diluted in PBS to 4× the EC80) is added to the respective wells. For the positive control, NetB only was used. The negative control contained no NetB. The NetB and VHH clones (35 uL total volume) are incubated in the assay plate for 15 minutes at room temperature prior the addition of 35 μL of washed and prepared RBCs. The assay plate is sealed with an adhesive plate seal, and then incubated at 37° C. for 30 mins, shaking (450 rpm). After incubation, the plate is centrifuged at 1100×g for 3 mins and 50 ul of each of the samples is removed to a suitable flat-bottom plate, being careful not to disturb the RBC pellet. The OD405 is recorded for the plate by a spectrophotometer.

### Results & Discussion

The data (see FIG. 2A) clearly show while some VHH are readily degraded after incubation with Chymotrypsin (as judged by the presence of multiple degradation bands, see for example Net 11, Net 15, Net 17 and Net 74), some remain largely intact and, when tested, are still able to inhibit NetB-induced lysis of red blood cells (see FIG. 2B and Table 5). In the gels shown in this (and other of the Examples herein), the VHH band appears at around 15 kDa.

TABLE 5
Activity of VHH after incubation with Chymotrypsin

| | IC50 after 1 ug/mL |
|---|---|
| | chymotrypsin |
| Clone ID | incubation (nM) |
| Net_07 | 204 |
| Net_14 | 114 |
| Net_30 | 131 |
| Net_83 | 195 |

### Example 5: Stability and Activity after Incubation with Pepsin (Resistance to Pepsin)

Candidate VHH were incubated with Pepsin over a time course up to 45 mins. After Pepsin incubation the reactions were stopped and the VHH were run on an SDS-PAGE gel and assessed for their continued ability to inhibit NetB induced red blood cell lysis.

### Material and Methods

Pepsin (Sigma Aldrich; cat no. P7125) was made up at 5 mg/mL (5360 U/mL) in PBS pH 1.5 (pH reduced to 1.5 using concentrated HCl). Pepsin was further diluted to 300 U/mL in PBS pH1.5) To maintain maximal pepsin protease activity, the pH in the assay must be below 2.

All VHH were prepared at 30 uM in 10 uL PBS pH 7.4 before the addition of 8 μL PBS pH1.5 to yield 18 μL of VHH at 16.67 uM. To this 2 μL of pepsin at 300 U/mL in PBS pH1.5 is added to yield a final reaction of: 15 uM VHH and 30 U/ml pepsin in PBS at pH 1.8. The tubes are then spun down for 15 seconds to ensure pepsin is added at the same time for all tubes. PCR tubes are then transferred to a heat block at 37° C., and 1 tube for each VHH is removed at 5, 15, 30 and 45 minutes. When removed from the heat block, the tubes are transferred immediately to ice to stop the digestion, before the addition of 1.2 μL 0.1M NaOH to bring the pH back up to 7.4, inactivating the pepsin. The samples are stored on ice until they are run on an SDS-PAGE gel, or used in the chRBC lysis screening assay.

As a further test with selected clones, the preparation of VHH and incubation with pepsin was done as above, but with incubation times extended out to 120 mins, with samples taken at 5, 30, 45, 60, 90, and 120 mins.

SDS-PAGE Gel of VHH after Incubation with Pepsin

5 μL of each stopped VHH sample is taken and added to 2 μL of 5×SDS loading buffer added to each tube, and the tubes sealed with a foil adhesive plate seal. The tubes are transferred to a heat block and incubated at 80° C. for 10 minutes before being spun down to collect any evaporate. The denatured samples are run out on a 4-20% graduated SDS-PAGE gel. After the gel has run, it is stained with Coomassie staining reagent for 24 h, then destained with water before pictures are taken to assess degradation after digestion with pepsin.

NetB-Induced Red Blood Cell Assay with VHH Incubated with Pepsin

10 μL of each stopped VHH sample is taken and added to 65 μL PBS, before being diluted 1 in 3 from 2 uM down to 5.49 nM. These dilutions are at 4× final concentration to allow for dilution upon addition of chRBCs and NetB toxin. NetB will be used at the sub-maximal concentration (EC80) of 20 nM. The maximum final concentration of VHH will be 500 nM=25:1 (VHH:NetB) molar ratio, and the lowest concentration will be 1.37 nM=0.0685:1 molar ratio (VHH:NetB)

VHH are diluted from 2 uM down to 5.49 nM in 3-fold serial dilutions and 17.5 uL transferred into a v-bottom 96 well assay plate. Then 17.5 uL NetB (diluted in PBS to 4× the EC80) is added to the respective wells. For the positive control, NetB only was used. The negative control contained no NetB. The NetB and VHH clones (35 uL total volume) are incubated in the assay plate for 15 minutes at room temperature prior the addition of 35 μL of washed and prepared RBCs. The assay plate is sealed with an adhesive plate seal, and then incubated at 37° C. for 30 mins, shaking (450 rpm). After incubation, the plate is centrifuged at 1100×g for 3 mins and 50 ul of each of the samples is removed to a suitable flat-bottom plate, being careful not to disturb the RBC pellet. The OD405 is recorded for the plate by a spectrophotometer.

### Results & Discussion

The data clearly show while some VHH are readily degraded after incubation with pepsin (as judged by the presence of multiple degradation bands and the loss of the 15 kDa VHH band, see for example Net 7 and Net 30 in FIGS. 3A and 3B), some remain largely intact and still able to inhibit NetB-induced lysis of red blood cells, even after 120 mins incubation with 30 U/ml pepsin. For example, the 15 kDa band is still present for Net 83 at 120 minutes (see FIG. 3C), and 250 nM of Net 83 is still showing 35% inhibition of NetB at 60 and 90 minutes (see FIG. 3D). Net 14 also still shows 45% inhibition of NetB at 45 and 60 minutes (see FIG. 3D). Net 7 performs much less well in terms of pepsin resistance. The IC50s of various clones in the NetB lysis assay are shown in Table 6.

TABLE 6
Activity of VHH after incubation with Pepsin

| | Resistance | | | | |
|---|---|---|---|---|---|
| | to Pepsin | Inhibition of NetB-induced RBC lysis at various | | | |
| Clone | (from SDS- | timepoints of incubation with Pepsin IC50 (nM) | | | |
| ID | PAGE gel) | 0 | 5 mins | 15 mins | 30 mins |
| Net_07 | − | 62 | 497 | 724 | nd |
| Net_13 | ++ | 105 | 180 | 459 | 541 |
| Net_14 | ++ | 23 | 109 | 124 | 113 |
| Net_30 | + | 33 | 183 | 624 | no |
| Net_83 | +++ | 61 | 179 | 231 | 208 |

### Example 6: Stability and Activity after Incubation with Pancreatin (Resistance to Pancreatin)

Candidate VHH were incubated with increasing concentrations of Pancreatin for 30 min, with samples run on SDS-PAGE gel to assess resistance to degradation. A selected clone (Net 83) was further incubated with pancreatin over a time course. After Pancreatin incubation the reactions were run on an SDS-PAGE gel and assessed for the extent of degradation.

### Material and Methods

Incubation of VHH with Different Concentrations of Pancreatin

VHH were prepared at a final conc. of 30 uM incubated with Pancreatin (8×USP specifications) (Sigma Aldrich; cat no. P7545-25G) at: 0, 0.1, 0.5, 1, 5 mg/ml final pancreatin concentration in 50 mM Tris-HCl pH 6.8+10 mM CaCl2. Samples were incubated for 30 mins at 37° C., before the reaction was stopped by heating the samples (95° C. for 3 mins) in SDS loading buffer. Samples were run on a gel with a no VHH control group to see the background from the Pancreatin

Incubation of Select Clone with Pancreatin, Time Course

Pancreatin was made up at 25 mg/mL in 1 mM HCl (pH3.0) and put on the roller for 90 mins.

After 60 mins, the pancreatin was diluted to 1 mg/mL in assay buffer (50 mM Tris-HCl pH 6.8+10 mM CaCl2) and kept on ice. VHH was diluted to 15 μM in assay buffer and placed on ice.

Pancreatin and VHH were added together at a 1:1 ratio, yielding a final concentration of 0.5 mg/mL pancreatin and 7.5 μM Net_83 and the tubes placed on the heat block at 37° C.

After 15, 30, 45, 60, 90 and 120 mins 20 μL was removed from the tube, 4 μL 5×SDS loading buffer added, and the samples denatured at 99° C. for 3 mins before being frozen.

Once all the samples were digested, denatured and frozen, they are run out on SDS-PAGE.

### Results & Discussion

The data (see FIG. 4A and FIG. 4B) clearly show the selected VHH (Net 83) remains largely intact after incubation with various concentrations of pancreatin, and even after 90 mins incubation with 0.5 mg/ml pancreatin.

### Example 7: VHH Stability in Water

### Material and Methods

### VHH Incubation in Water

VHH were buffer exchanged into water and diluted to 50 ug/ml (Net 14) or 100 ug/ml (Net_07 and Net_83) in water and incubated at room temperature. At various timepoints samples were taken and frozen at −80 C until the completion of the study, when samples were run on a gel or tested for inhibition of NetB-induced red blood cell lysis.

### Preparation of VHH for the Inhibition of NetB-Induced Red Blood Cell Lysis

VHH are diluted from 4 uM down to 5.49 nM in 3-fold serial dilutions and 17.5 uL transferred into a v-bottom 96 well assay plate. Then 17.5 uL NetB (diluted in PBS to 4× the EC80) is added to the respective wells. For the positive control, NetB only was used. The negative control contained no NetB. The NetB and VHH clones (35 uL total volume) are incubated in the assay plate for 15 minutes at room temperature prior the addition of 35 μL of washed and prepared RBCs. The assay plate is sealed with an adhesive plate seal, and then incubated at 37° C. for 30 mins, shaking (450 rpm). After incubation, the plate is centrifuged at 1100×g for 3 mins and 50 ul of each of the samples is removed to a suitable flat-bottom plate, being careful not to disturb the RBC pellet. The OD405 is recorded for the plate by a spectrophotometer.

### Results and Discussion

As can be seen in FIG. 5A, the VHH remain unchanged over the time course: the 15 kDa band is still present after 4 days, with no significant signs of degradation.

FIG. 5B and Table 7 both show that the VHH show no loss of potency throughout the time course.

This time course was extended until 28 days and stability of the VHH was still observed, including in the NetB functional assay.

TABLE 7
Stability of VHH in water

| Clone | IC50 after incubation in water (nM) | | |
|---|---|---|---|
| ID | Day 0 | Day 1 | Day 4 |
| Net_14 | 44.23 | 52.95 | 51.1 |
| Net_83 | 163.4 | 168.1 | 244.7 |

### Example 8: Affinity Determination of VHH for NetB Toxin (KD by Biacore)

### Material and Methods

The affinity of binding for Clostridium perfringens NetB toxin by each of the identified VHH antibody leads was determined by surface plasmon resonance.

### KD Determination by Biacore

To assess the affinity of selected purified clones to NetB toxin, NetB toxin protein was coated by amine coupling on a CM5 sensor ship (Cytiva Cat. nr. 29149603). Surface plasmon resonance (SPR) (Biacore 8K+, Cytiva) in single cycle kinetics (SCK) was used to determine the binding kinetics of selected VHH at pH7.4. Approximately 350 RU of NetB toxin in Acetate buffer pH 5.0 or pH 5.5 were immobilized onto a CM5 chip using the standard amine coupling procedure. QC of the immobilization was done using a commercial antibody anti-Histidine (BioLegend; Cat. nr. 652505) at 5 nM and 30 nM diluted in 1×HBS-EP+pH 7.4 buffer. 1×HBS-EP pH 7.4 was utilized as a running buffer during binding kinetic measurements. Purified VHH were injected in two-fold serial dilutions starting at 20 nM down to 1.3 nM in 1×HBS-EP+pH 7.4, at 30 μl/min for 2 minutes followed by off-rate wash for 1 min between VHH injections. The final off-rate wash was 5 min after the final VHH injection in each cycle.

Affinity and RU max were calculated using the using the Single Cycle Kinetics predefined evaluation method of the Biacore Insight Evaluation Software.

### Results & Discussion

As can be seen in Table 8 and FIG. 6, KD values for selected VHH were 0.095 nM for Net14 and 0.213 nM for Net83.

TABLE 8
Summary of Biacore affinity data

| | Chi² | Ka | Kd | Rmax | KD |
|---|---|---|---|---|---|
| VHH | (RU²) | (1/Ms) | (1/s) | (RU) | (nM) |
| Net_14 | 0.03 | 6.68E+05 | 6.32E−05 | 57 | 0.095 |
| Net_83 | 0.05 | 1.17E+06 | 2.48E−04 | 62 | 0.213 |

### Example 9: Efficacy Study of Chicken Necrotic Enteritis Model

The objective of the study was to investigate the safety and efficacy of Net_83 in combination with an antibody against C. perfringens necrotic enteritis alfa (CPA) toxin after experimental induction of necrotic enteritis (NE) in broilers. The antibody against CPA was a VHH antibody provided by ECO Animal Health Ltd.

### 1. Groups Description

On study day 1 a total of 810 Ross 308 one day old chicks were allocated to pens of 15 birds each. Pens were subsequently randomised to one of the following treatment groups: T01=infected untreated control (IUC), T02=unchallenged untreated (UUC), T03=Amoxicillin in drinking water for 5 days, T04=CPA+Net_83 antibodies in drinking water for 6 days, T05=NaHCO3 in drinking water for 6 days and T06=CPA+Net_83 antibodies in water with NaHCO3 for 6 days. Each group had 10 pens except for T02 that had 4 pens. On challenge initiation, the number of birds per pen was reduced to 13 resulting in a total of 702 birds included in the evaluation. All groups except T02 were challenged as described further below. In water treatment started the day before first C. perfringens challenge. A summary of the groups is presented in Table 9 below.

TABLE 9
Description of the different treatment groups.

| | | Treatment | Administration | Total number | Replicates | Birds per |
|---|---|---|---|---|---|---|
| Group | Treatment* | regimen | route | of birds | (pens) | replicate |
| T01 (IUC) | No | — | — | 130 | 10 | 13 |
| T02 (UUC) | No | — | — | 52 | 4 | 13 |
| T03 | Amoxicillin | d 17 to d 21 | ad libitum | 130 | 10 | 13 |
| | (13.1 mg/kg) | (5 d) | in water | | | |
| T04 | CPA + Net_83 | d 17 to d 22 | ad libitum | 130 | 10 | 13 |
| | (5 mg of | (6 d) | In water | | | |
| | each/bird/day) | | | | | |
| T05 | NaHCO3 | d 17 to d 22 | ad libitum | 130 | 10 | 13 |
| | (0.5 g/L) | (6 d) | in water | | | |
| T06 | CPA + Net_83 + | d 17 to d 22 | ad libitum | 130 | 10 | 13 |
| | NaHCO3 | (6 d) | In water | | | |
| | (5 mg of | | | | | |
| | each VHH/bird/ | | | | | |
| | day + 0.5 g/L) | | | | | |

*Birds received the corresponding test item and were challenged with coccidia and C. perfringens (see challenge description further below).

### 2. Chicken Necrotic Enteritis Model

Necrotic enteritis was induced by a combination of predisposing factors. On day 14 and 16 a 10-fold overdose of a commercially available Eimeria vaccine (to provide a coccidia challenge) was given by gavage. On day 17, the feed was switched to a grower feed with 30% fish meal. From day 18 onwards, an alpha toxin-producing and NetB-positive C. perfringens strain was given via oral gavage for four consecutive days. Birds were followed until 30 days of age.

### Treatment Administration

### 2.1. Feasibility of Net_83 Administration in Water

Prior to the efficacy study, feasibility of water administration of Net_83 antibody was evaluated in a pilot study. The objective of that study was to evaluate if Net_83 reaches the gastrointestinal tract of birds when administered in the drinking water either alone (T01-1) or in combination with NaHCO3(T02-1). Each treatment was administered adlibitum during 24 hours to one pen housing 10 birds. Each pen had a water line mimicking those in commercial farms. Digesta samples were collected 24 hours after administration started to analyse the presence of Net_83 in the crop by ELISA. The antibody could be retrieved from the crop samples in comparable amounts in both treatment groups (FIG. 7).

This study demonstrates that the antibody is stable under these administration conditions and that this is an appropriate way to administer the antibody to the birds as the presence in the crop indicates that the antibody reaches the digestive tract of the birds using this method.

### 2.2 Treatment Administration in the Efficacy Study

The treatments were administered in drinking water offered adlibitum.

Birds in T03 were given a commercially available water soluble formulation of amoxicillin in drinking water. The target dose was 13.1 mg amoxicillin per kg body weight for 5 consecutive days, as per label recommendation. Amoxicillin treatment was administered preventively starting 1 day prior to C. perfringens challenge and continued during five days (study days 17, 18, 19, 20 and 21).

Birds in T04 and T06 received water supplemented with a combination of Net_14 and CPA antibodies at the same concentration. This water treatment was provided from day 17 to day 22 at a dose rate of 5 mg/bird/day for each antibody. Antibodies were mixed in the water that a pen would consume in one day as follows:
1. The vials containing CPA and Net_83 antibodies were gently homogenized by inverting.
2. The content of each vial was mixed into the volume of water added to the corresponding pen for that day.

The Net_83 and CPA antibodies were supplied as a ready to use solution to be mixed in drinking water.

Water was prepared daily per pen using tap water. For T06, a stock solution of water with 0.5 g/L sodium bicarbonate was prepared before adding the antibodies. The amount of water to be added was based on expected daily water intake (DWI). The amount was calculated for each pen based on the number of birds in that pen and the daily feed intake according to Ross308 standards 2018. The assumption was that the bird water intake is twice the feed intake. The target concentration of each antibody in water (mg/mL) to achieve a 5 mg/bird dose was calculated accounting for this estimated water intake.

### 3. Efficacy Evaluation

The primary variable was NE-related mortality.

The necrotic enteritis challenge was successful as confirmed by high NE-related mortality in the TO1 (IUC) (79%). This mortality was higher than expected resulting in low statistical power. Initial survival analyses showed that both groups treated with antibodies (T04 and T06) performed very similarly (FIG. 8). The analyses also showed that NaHCO₃ did not have an effect on mortality and could be considered as a placebo (FIG. 8).

Because no effect of NaHCO₃ on mortality was observed, and in order to increase statistical power, additional analyses were conducted on pooled data of TO1 (IUC) and T05 (NaHCO₃) groups (untreated birds) compared to pooled data of groups treated with antibodies (T04&T06).

Survival analyses on pooled data showed a significant reduction in the probability of death (71%) for birds treated with antibodies compared to untreated birds (77%; P=0.038; FIG. 9).

Due to the severity of the challenge, several pens lost all birds. An additional survival analysis was conducted to compare the probability of a pen of losing all birds in treated vs. untreated groups. Analyses of pooled data showed that the number of pens that lost all birds due to NE was less in the birds treated with antibodies compared to the pooled data of the IUC and the NaHCO₃ groups (45% vs 75%; P=0.048; FIG. 10). This is an indication that in the field, units treated with these VHHs will be less severely affected by NE.

No adverse events related to the administered products was reported.

In conclusion, the NE mortality induced in this study was higher than expected and the statistical power was low. Also, severe challenge resulted in reduced water consumption and the target antibody dose was not achieved on some treatment days. Nonetheless, additional analyses conducted on pooled data showed that, even under these severe challenge conditions (which are expected to be much more severe than conditions seen in the field when birds are exposed naturally to these NE inducing pathogens), the Net_83+CPA antibody combination achieved a statistical reduction of mortality compared to untreated birds.

### Example 10: Net_83 and Net_14 Expression in Bacillus subtilis as Vector for Oral Delivery

Bacillus subtilis strain (PY79, obtained from Bacillus Genetic Stock Center, BGSID No: 1A747) was genetically engineered to express either Net_14 or Net_83. For each construct, the antibody was cloned into the amyE locus of PY79 with a chloramphenicol (Cm) resistance selection marker as described elsewhere (Yang et al., 2020). Bacterial culture supernatants were collected and submitted to ELISA to confirm expression of binding antibody as described below.

Briefly NetB-His toxin was coated in a Maxisorp plate at, 2 μg/ml in 1×PBS, 4° C., O/N. The coated plate was blocked with 4% skimmed milk in 1×PBS, 1 h, RT. The supernatant of three Bacillus subtilis clones expressing Net_14, three expressing Net_83 and two expressing an irrelevant VHH clone (Anti-JTT) were titrated by performing 7-steps, serial dilutions, starting at a 1:2 dilution in 1% skimmed milk in 1×PBS.

Antibodies were detected with a goat anti-VHH antibody (Jackson ImmunoResearch, cat. No. 128-005-230) at 1:500 dilution in 1% skimmed milk in 1×PBS.

The donkey anti-goat-HRP antibody (Jackson ImmunoResearch, cat. No. 705-035-147) at 1:5000 dilution in 1% skimmed milk in 1×PBS, was used as secondary detection.

Coating control was performed with Anti-Histidine-HRP (Miltenyi Biotec, Cat. nr. 130-092-783) at 1:500 in 1% skimmed milk in 1×PBS

Binding to NetB-His was assessed by measuring O.D. at 450 nm

### Results and Conclusions

The supernatant of all three B. subtilis clones, expressing Net_14, showed positive binding to NetB toxin down to 1:100000 dilution (FIG. 11), and each supernatant should contain between 15-19 μg/mL of Net_14 VHH

The supernatant of all three B. subtilis clones, expressing Net_83, showed positive binding to NetB toxin from 1:2 to 1:1000 dilution (FIG. 12), and each supernatant should contain ˜4 μg/mL of Net_83 antibody.

It can be concluded that B. subtillis is an appropriate vector for Net_14 and Net_83 antibodies as it can be used to express functional antibodies in the supernatant that bind to the target NetB toxin. In other experiments these B. subtillis secreted antibodies have also been shown to retain the function of inhibiting NetB induced chRBC lysis.

### REFERENCE

Yang M, Zhu G, Korza G, Sun X, Setlow P, Li J. 2020. Engineering Bacillus subtilis as a versatile and stable platform for production of nanobodies. Appl Environ Microbiol 86:e02938-19.

1. A binding protein comprising at least one antigen binding domain which binds to necrotic enteritis B-like toxin (NetB), said antigen binding domain comprising a heavy chain variable region which comprises three complementarity determining regions (CDRs), wherein said binding protein binds to NetB with a KD of 250 pM or less at pH 7.4.

2. The binding protein of claim 1, wherein said binding protein is capable of reducing the toxic activity of NetB at a pH of 6.0.

3. The binding protein of claim 1 or claim 2, wherein said binding protein is capable of reducing the toxic activity of an EC₈₀ concentration of NetB or 20 nM NetB against chicken red blood cells with an IC₅₀ value of less than 150 nM at a pH of 6.0.

4. The binding protein of any one of claims 1 to 3, wherein said binding protein shows resistance to pepsin digestion at a pepsin concentration of 30 U/ml and a temperature of 37° C.

5. The binding protein of any one of claims 1 to 4, wherein said binding protein shows resistance to chymotrypsin digestion at a chymotrypsin concentration of 1 μg/ml and a temperature of 37° C.

6. The binding protein of any one of claims 1 to 5, wherein said binding protein shows resistance to pancreatin digestion at a pancreatin concentration of 0.5 mg/ml and a temperature of 37° C.

7. The binding protein of any one of claims 1 to 6, wherein said heavy chain variable region comprises:
(i) a variable heavy (VH) CDR1 that comprises the amino acid sequence of GSIRSISFMN (SEQ ID NO:2), or a sequence substantially homologous thereto, wherein said substantially homologous sequence is a sequence containing 1, 2, 3 or 4 amino acid substitutions compared to the given CDR sequence,
(ii) a variable heavy (VH) CDR2 that comprises the amino acid sequence of INQIGNT (SEQ ID NO:3), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of IVRGTVSPFQF (SEQ ID NO:4), or a sequence substantially homologous thereto, wherein said substantially homologous sequence is a sequence containing 1, 2, 3 or 4 amino acid substitutions compared to the given CDR sequence.

8. The binding protein of any one of claims 1 to 6, wherein said heavy chain variable region comprises:
(i) a variable heavy (VH) CDR1 that comprises the amino acid sequence of GLTFSGYSLG (SEQ ID NO:10), or a sequence substantially homologous thereto, wherein said substantially homologous sequence is a sequence containing 1, 2, 3 or 4 amino acid substitutions compared to the given CDR sequence,
(ii) a variable heavy (VH) CDR2 that comprises the amino acid sequence of ISSSGMIT (SEQ ID NO:11), or a sequence substantially homologous thereto, wherein said substantially homologous sequence is a sequence containing 1, 2 or 3 amino acid substitutions compared to the given CDR sequence, and
(iii) a variable heavy (VH) CDR3 that comprises the amino acid sequence of RFPRPRSWLSTDSYNY (SEQ ID NO:12), or a sequence substantially homologous thereto, wherein said substantially homologous sequence is a sequence containing 1, 2, 3 or 4 amino acid substitutions compared to the given CDR sequence.

9. The binding protein of any one of claims 1 to 8, wherein said binding protein is or comprises an antibody.

10. The binding protein of claim 9, wherein said antibody is a single domain antibody.

11. An antibody that binds to the same epitope of NetB as an antibody with a heavy chain variable region as defined in claim 7 or claim 8.

12. The binding protein or antibody of any one of claims 1 to 11, wherein said binding protein or antibody is provided as a dimer or larger multimer.

13. One or more nucleic acid molecules comprising nucleotide sequences that encode a binding protein or antibody of any one of claims 1 to 12.

14. One or more expression vectors comprising the one or more of the nucleic acid molecules of claim 13.

15. One or more host cells comprising said expression vectors of claim 14, or said nucleic acid molecules of claim 13, or expressing the antibody or binding protein of any one of claims 1 to 12.

16. The host cells of claim 15, wherein said cells are bacterial cells, preferably probiotic bacterial cells, or yeast cells.

17. The host cells of claim 16, wherein said bacterial cells are Bacillus, preferably Bacillus subtilis, Lactobacillus, preferably Lactobacillus reuteri, Lactococcus, Salmonella, Escherichia coli or Listeria, or wherein said yeast cells are Pichia or Saccharomyces.

18. A method of producing a binding protein or antibody of any one of claims 1 to 12, said method comprising the steps of (i) culturing a host cell of any one of claims 15 to 17, under conditions suitable for the expression of the encoded binding protein or antibody; and optionally (ii) isolating or obtaining the binding protein or antibody from the host cell or from the growth medium/supernatant.

19. A composition, preferably a pharmaceutically acceptable composition, comprising a binding protein or antibody of any one of claims 1 to 12, the one or more nucleic acid molecules of claim 13, the one or more expression vectors of claim 14, or the one or more host cells of claims 15 to 17.

20. The composition of claim 19, comprising further active agents.

21. The composition of claim 20, wherein a further active agent is an agent that inhibits C. perfringens alpha-toxin (CPA), preferably an anti-CPA antibody.

22. A food product or food additive, preferably an animal feed product or additive, comprising a binding protein or antibody of any one of claims 1 to 12, or one or more host cells of any one of claims 15 to 17, or a composition of any one of claims 19 to 21.

23. The food product or food additive of claim 22, wherein said product or additive is a feed premix or a top dressing powder.

24. The binding protein or antibody of any one of claims 1 to 12, the one or more nucleic acid molecules of claim 13, the one or more expression vectors of claim 14, the one or more host cells of claims 15 to 17, or the composition of any one of claims 19 to 21, for use in therapy in a subject, preferably for use in the treatment or prevention of C. perfringens infection in a subject.

25. The binding protein or antibody of any one of claims 1 to 12, the one or more nucleic acid molecules of claim 13, the one or more expression vectors of claim 14, the one or more host cells of claims 15 to 17, or the composition of any one of claims 19 to 21, for use in the treatment or prevention of Necrotic enteritis (NE) in a subject.

26. Use of the binding protein or antibody of any one of claims 1 to 12, the one or more nucleic acid molecules of claim 13, the one or more expression vectors of claim 14, the one or more host cells of claims 15 to 17, or the composition of any one of claims 19 to 21, in the manufacture of a medicament or composition for use in the treatment or prevention of C. perfringens infection in a subject.

27. Use of the binding protein or antibody of any one of claims 1 to 12, the one or more nucleic acid molecules of claim 13, the one or more expression vectors of claim 14, the one or more host cells of claims 15 to 17, or the composition of any one of claims 19 to 21, in the manufacture of a medicament or composition for use in the treatment or prevention of Necrotic enteritis (NE) in a subject.

28. A method of treatment or prevention of C. perfringens infection in a subject, wherein said method comprises the step of administering to a subject in need thereof a therapeutically effective amount of the binding protein or antibody of any one of claims 1 to 12, the one or more nucleic acid molecules of claim 13, the one or more expression vectors of claim 14, the one or more host cells of claims 15 to 17, or the composition of any one of claims 19 to 21.

29. A method of treatment or prevention of Necrotic enteritis (NE) in a subject, wherein said method comprises the step of administering to a subject in need thereof a therapeutically effective amount of the binding protein or antibody of any one of claims 1 to 12, the one or more nucleic acid molecules of claim 13, the one or more expression vectors of claim 14, the one or more host cells of claims 15 to 17, or the composition of any one of claims 19 to 21.

30. The binding protein, antibody, nucleic acid molecules, expression vectors, host cells or compositions for use of claim 24 or claim 25, the use of claim 26 or claim 27, or the method of claim 28 or claim 29, wherein said subject is an avian species, preferably poultry, more preferably a chicken.


SEQUENCE LISTING (18 sequences)

SEQ ID NO: 1 - protein, 119 aa, synthetic construct
QVQLVESGGGLVQAGESLRLSCAASGSIRSISFMNWYRQQPGKQRELVAGINQIGNTNYAGSVKGRFTISRDNAKNTVYLQMNSLKPEDTAVYYCNVIVRGTVSPFQFWGQGTQVTVSS

SEQ ID NO: 2 - protein, 10 aa, synthetic construct
GSIRSISFMN

SEQ ID NO: 3 - protein, 7 aa, synthetic construct
INQIGNT

SEQ ID NO: 4 - protein, 11 aa, synthetic construct
IVRGTVSPFQF

SEQ ID NO: 5 - protein, 25 aa, synthetic construct
QVQLVESGGGLVQAGESLRLSCAAS

SEQ ID NO: 6 - protein, 15 aa, synthetic construct
WYRQQPGKQRELVAG

SEQ ID NO: 7 - protein, 40 aa, synthetic construct
NYAGSVKGRFTISRDNAKNTVYLQMNSLKPEDTAVYYCNV

SEQ ID NO: 8 - protein, 11 aa, synthetic construct
WGQGTQVTVSS

SEQ ID NO: 9 - protein, 125 aa, synthetic construct
QVQLVESGGGLVQAGGSVRLSCVASGLTFSGYSLGWFRQAPGKEREGVSYISSSGMITDYADSVKGRVTISRDDAKSTMYLQMNSLKPEDTAVYYCAARFPRPRSWLSTDSYNYWGQGAQVTVSS

SEQ ID NO: 10 - protein, 10 aa, synthetic construct
GLTFSGYSLG

SEQ ID NO: 11 - protein, 8 aa, synthetic construct
ISSSGMIT

SEQ ID NO: 12 - protein, 16 aa, synthetic construct
RFPRPRSWLSTDSYNY

SEQ ID NO: 13 - protein, 25 aa, synthetic construct
QVQLVESGGGLVQAGGSVRLSCVAS

SEQ ID NO: 14 - protein, 15 aa, synthetic construct
WFRQAPGKEREGVSY

SEQ ID NO: 15 - protein, 40 aa, synthetic construct
DYADSVKGRVTISRDDAKSTMYLQMNSLKPEDTAVYYCAA

SEQ ID NO: 16 - protein, 11 aa, synthetic construct
WGQGAQVTVSS

SEQ ID NO: 17 - protein, 292 aa, Clostridium perfringens
SELNDINKIELKNLSGEIIKENGKEAIKYTSSDTASHKGWKATLSGTFIEDPHSDKKTALLNLEGFIPSDKQIFGSKYYGKMKWPETYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQWRGTNKLSSTSEYNEFMFKINWQDHKIEYYL

SEQ ID NO: 18 - protein, 292 aa, synthetic construct
SELNDINKIELKNLSGEIIKENGKEAIKYTSSDTASHKGWKATLSGTFIEDPHSDKKTALLNLEGFIPSDKQIFGSKYYGKMKWPETYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQARGTNKLSSTSEYNEFMFKINWQDHKIEYYL